(19) United States
(12) Patent Application Publication
(10) Pub. No.: US 2025/0222119 A1
(43) Pub. Date: Jul. 10, 2025

(54) ENGINEERING OF MULTIFUNCTIONAL PEPTIDES FOR CONTROLLED DRUG DELIVERY

(71) Applicants: The Johns Hopkins University, Baltimore, MD (US); University Of Maryland, College Park, College Park, MD (US)

(72) Inventors: Laura Ensign, Towson, MD (US); Tung Heng Henry Hsueh, Baltomore, MD (US); Justin Hanes, Baltimore, MD (US); Michael P. Cummings, Washington, DC (US); Renee Ti Chou, Laurel, MD (US)

(21) Appl. No.: 18/864,859

(22) PCT Filed: May 11, 2023
(86) PCT No.: PCT/US2023/066895

§ 371 Date: Nov. 11, 2024

Related U.S. Application Data
(60) Provisional application No. 63/340,714, filed on May 11, 2022.

(51) Int. Cl. A61K 47/64; A61K 9/00; A61K 9/06; A61K 31/404; A61K 47/10; A61P 27/02; C07K 7/08; G01N 33/50; G01N 33/543; G01N 33/68

(52) U.S. Cl. CPC A61K 47/64; A61K 9/0048; A61K 9/06; A61K 31/404; A61K 47/10; A61P 27/02; C07K 7/08; G01N 33/5014; G01N 33/502; G01N 33/543; G01N 33/6845; G01N 2410/00

(57) ABSTRACT

Methods of designing, generating, screening, and identifying one or more of these peptides possessing multiple functionalities, for example, including (i) high binding to a cellular target (e.g., melanin pigment in the eye), (ii) high cell-penetration (e.g., to enter cells and access melanin in the melanosomes), and (ii) low cytotoxicity have been developed. Peptides or drug conjugates thereof having these multiple functionalities are particularly effective for selective delivery, prolonged residence, and sustained release of active agents to the epithelial tissues via mucosal surfaces. Methods of using these peptides are also described.

### CROSS-REFERENCE TO RELATED APPLICATION

This application claims the benefit of and priority to U.S. Ser. No. 63/340,714 filed May 11, 2022, and which is incorporated by referenced herein in its entirety.

### STATEMENT REGARDING FEDERALLY SPONSORED RESEARCH OR DEVELOPMENT

This invention was made with government support under grants EY026578 and EY031041 awarded by the National Institutes of Health. The government has certain rights in the invention.

### REFERENCE TO SEQUENCE LISTING

The Sequence Listing submitted as an xml file named “JHU_C_17191_PCT_ST26.xml,” created on May 11, 2023, and having a size of 1,958 bytes is hereby incorporated by reference pursuant to 37 C.F.R. § 1.834(c)(1).

### FIELD OF THE INVENTION

This invention is generally in the field of peptides for use in making drug conjugates and formulations thereof for enhanced drug delivery, in particular drug delivery at epithelial surfaces such as the surface of the eye.

### BACKGROUND OF THE INVENTION

The eye is a very complex structure, with unique anatomy and physiology. The anatomy of the interior of the eye includes the anterior (front) segment, which occupies approximately one third of the total area of the eye, and posterior (rear) segment, which constitutes the remaining two thirds of the area. Tissues such as the cornea, conjunctiva, aqueous humor, iris, ciliary body, and lens make up the anterior portion, whilst the posterior segment includes the sclera, choroid, retinal pigment epithelium, neural retina, optic nerve, and vitreous humor.

Both the anterior and posterior segments of the eye are affected by various ophthalmic diseases and disorders, many of which are vision-threatening, including glaucoma, allergic conjunctivitis, anterior uveitis and cataract, age-related macular degeneration (AMD), and diabetic retinopathy. Whilst many therapeutic agents for treating ophthalmic disease are available, the delivery of effective amounts of drugs to the interior of the eye often requires uncomfortable and potentially dangerous invasive techniques, such as intra-ocular injection.

The most common methods for delivering ocular drugs are eyedrops and injections (Kompella, et al., Prog Retin Eye Res 82, 100901, (2021); Liebmann, et al. J Glaucoma 29 Suppl 1, S1-57, (2020); Kim, et al. Nat Biomed Eng 4, 1053-1062, (2020); Patel, et al. World J Pharmacol 2, 47-64, (2013)). Eyedrops have the challenge of providing limited drug penetration into the eye, often necessitating many drops per day to be effective. As the requirement for eyedrop dosing frequency increases, patient compliance decreases, a major issue for disease management. The inefficiency of drug delivery via eyedrops also precludes the use of eyedrops for treating retinal diseases (Rodrigues, et al. Pharm Res 35, 245, (2018); Iwase, et al. Invest Ophthalmol Vis Sci 54, 503-511, (2013)).

With injections, needle penetration into the eye comes with risks of infection and other complications, and thus, the less frequent, the better. Further, in many cases, ocular diseases are chronic in nature and require treatment over years or decades (Jager, et al., Retina 24, 676-698, (2004); Boyer, et al., Ophthalmology 121, 1904-1914, (2014); Black, et al., J AAPOS 13, 558-562, (2009); Fogagnolo & Rossetti, Expert Opin Investig Drugs 20, 947-959, (2011)).

Thus, for any drug delivery strategy, providing sustained therapeutic drug levels to decrease the required frequency of administration would improve disease management and patient quality of life. Sustained delivery of drugs through the mucosal surfaces of the eye has potential for improving the treatment and prevention of many ocular diseases, including infections, inflammatory disease, and degenerative eye conditions. However, achieving sustained prophylactic or therapeutic drug concentrations using traditional soluble dosage forms remains challenging due to degradation, rapid shedding, and rapid systemic absorption of drug.

The use of gels that coat the eye for drug delivery has been described (see, for example, U.S. publication Nos. 2021/0196837 and 2021/0177751) but has limitations. Various nano-formulations have also been introduced for anterior segment ocular drug delivery, and drug releasing devices and nano-formulations are being developed for posterior ocular delivery, for example, for treating chronic vitreoretinal diseases. However, many topical formulations have poor rheological properties, such as high tackiness, and poor viscosity, leading to discomfort and disruption of the gel upon blinking.

There remains an urgent need for enhanced delivery systems that provide long-lasting and effective drug delivery via the ocular surface without invasive, vision-disruptive, or uncomfortable procedures, particularly for the repeated or prolonged treatment of chronic diseases and disorders.

Therefore, there is an unmet need for systems and processes to design and engineer reagents for enhanced, prolonged delivery of drugs to mucosal surfaces.

It is an object of the present invention to provide design parameters and systems to engineer drug delivery compositions for increased retention time at the ocular surface.

It is a further object of the present invention to provide methods to engineer multifunctional peptides that can be chemically conjugated to drugs to enhance the pharmacokinetics and pharmacodynamics upon administration to the eye.

It is another object of the present invention to provide improved compositions for prolonged delivery to epithelial tissues through mucosal surfaces, which allow retention and sustained release of prophylactic, therapeutic or diagnostic agents to the epithelial tissues via mucosal surfaces.

### SUMMARY OF THE INVENTION

Methods and reagents enhancing retention time to drugs upon administration to the eye have been developed. A method to engineer multifunctional peptides that can be chemically conjugated to drugs to enhance the pharmacokinetics and pharmacodynamics upon administration to the eye has been established. The systems allow for designing peptide sequences that simultaneously provide multiple desired properties, including high binding to melanin (pigment in the eye), high cell-penetration (to enter cells and access melanin in the melanosomes), and low cytotoxicity.

Peptides for enhancing the delivery of an active agent to the eye are described. The peptides bind to melanin, penetrates cells and is non-toxic. In some embodiments, the peptides are between 5 and 12 amino acids, inclusive. In one embodiment, the peptide has the amino acid sequence FSGKRRKRKPR (SEQ ID NO:1).

Peptide conjugates for delivery of active agents to the eye are also described. The peptide conjugate typically includes: (a) a peptide which binds to melanin, penetrates cells and is non-toxic, for selective rapid delivery and retention of an active agent to the eye; and (b) an active agent. The active agent is conjugated to the peptide. In preferred embodiments, the amount of the active agent delivered to the eye by the peptide conjugate is greater than the amount of the active agent that is delivered to the eye in the absence of the peptide. In some embodiments, the peptides are between 5 and 12 amino acids, inclusive. In one embodiment, the peptide has the amino acid sequence FSGKRRKRKPR (SEQ ID NO:1). In some embodiments, the active agent is a therapeutic agent, prophylactic agent, and/or diagnostic agent. In other embodiments, the active agent is a protein, nucleic acid, carbohydrate, and/or small molecule. In further embodiments, the agent is a steroid, glaucoma agent, tyrosine kinase inhibitor, immunosuppressive agent, anti-fibrotic agent anti-infectives, antioxidant that prevents oxidative and/or nitrosative damage, hormone, and/or chemotherapeutic agent. In one embodiment, the active agent is a small molecule therapeutic agent. In preferred embodiments, the active agent is sunitinib, brimonidine, brinzolamide, cyclosporine A, moxifloxacin, budesonide, or acriflavine. In one preferred embodiment, the polypeptide includes the amino acid sequence FSGKRRKRKPR (SEQ ID NO:1), and the active agent is sunitinib.

Pharmaceutical formulations including the peptide conjugate and a pharmaceutically acceptable excipient for administration to the eye are also provided. In some embodiments, the pharmaceutical formulation is formulated for topical, intracameral, subconjunctival and intravitreal administration. In preferred embodiments, the pharmaceutical formulations include a gel-forming polymer for application to the eye, formulated so that it is at a concentration below the critical gel concentration (CGC) of the polymer under isotonic conditions at a temperature between about 25 to about 37 C, and excipients to form a pharmaceutically acceptable hypotonic formulation of the polymer suitable for delivery to the eye of an individual in need thereof. In one embodiment, the formulation is in dry or freeze-dried form. In some embodiments, the gel-forming polymer is a thermosensitive gel-forming polymer, for example, having a critical solution temperature that is below 30 C. In one embodiment, the polymer is a poloxamer. In preferred embodiments, the polymer in combination with the excipient forms a gel on administration into or onto the eye. In a preferred embodiments the gel-forming polymer is a poloxamer, for example, poly(ethylene glycol)-block-poly(propylene glycol)-block-poly(ethylene glycol) (PLURONIC® F127). In some embodiments, the gel-forming polymer is between 10% and 16% PLURONIC® F127. In some embodiments, the gel-forming polymer is between greater than 12% and less than 24% PLURONIC® F98 (ethylene oxide/propylene glycol propylene oxide condensate that functions as a non-ionic surfactant) in an aqueous excipient. In other embodiments, the gel-forming polymer is between 10 and 18% poloxamer F127. In some embodiments, the gel-forming polymer forms a uniformly thick layer at the time of administration onto the ocular surface.

Dosage forms having an effective amount of the pharmaceutical formulation including the peptide-active agent conjugate and a pharmaceutically acceptable excipient for administration to the eye are also provided. In preferred embodiments, the dosage is effective to retains an effective concentration of the active agent at the epithelial tissues inside the eye for more than one day, more than one week, more than one month or more than three months. In some embodiments, the dosage for administration is in the form of a dry powder, gel, or liquid.

Methods for treating an eye disease or disorder in a subject in need thereof are also provided. Methods include administering to the eye of the subject dosage forms having an effective amount of the pharmaceutical formulation including the peptide conjugate and a pharmaceutically acceptable excipient for administration to the eye. In some embodiments, the dosage form is administered by topical, intracameral, subconjunctival or intravitreal administration. Disease or disorders to be treated include glaucoma, dry eye syndrome, macular degeneration, diabetic retinopathy, scleroderma, and cancer. In some embodiments, the dosage form is administered as an eye drop into the eye of the subject, for example, in an amount between about 10 μl and about 100 μl, inclusive, of the formulation. In other embodiments, the dosage form is administered as an injection into the eye of the subject. In some embodiments, the method is repeated once or more. In some embodiments, the method is repeated at a time such as hourly, daily, every other day, every three days, every four days, every five days, every six days, weekly, every two weeks, or less often.

Methods of screening for peptides having multiple desired functional properties, include:
(i) screening the peptides for selective binding to a desired cellular target to identify one or more target-binding peptides;
(ii) generating a plurality of peptide variants based on the target binding peptides identified in (i) to create a first target-binding peptide library;
(iii) screening the first target-binding peptide library to identify a subset of target binding peptides;
(iv) screening the subset of target binding peptides for cell-penetration, and selecting a subset of cell-penetrating, target binding polypeptides; and
(v) screening the subset of cell-penetrating, target binding peptides for toxicity to identify a subset of non-toxic, cell-penetrating target binding polypeptides.

In some embodiments, screening the peptides for selective binding in step (i) includes using an in vitro binding assay including an enzyme-linked immunoassay (ELISA), a phage display library, or a microarray system such a high-throughput flow-based microarray system. In some embodiments, the plurality of peptide variants in step (ii) are randomly generated with a frequency of 5% for each of the 20 amino acids based on the target-binding peptides identified in (i). In one embodiment, the cellular target is melanin.

Methods of designing, generating, screening, and identifying one or more peptides possessing multiple functionalities including: (i) high binding to melanin (pigment in the eye); (ii) high cell-penetration (e.g., to enter cells and access melanin in the melanosomes); and (ii) low cytotoxicity are also described.

Also described is a peptide comprising the amino acid sequence FSGKRRKRKPR (SEQ ID NO:1), or one or more amino acid substitutions thereof.

### BRIEF DESCRIPTION OF THE DRAWINGS

FIGS. 1A-1D are schematics showing an overview of melanin binding peptide identification. FIG. 1A shows a pilot 119 peptide microarray analysis, with peptides (˜) anchored to a microarray (⋄), and melanin formulated into nanoparticles with surface biotinylation (b-mNPs) passed over the microarray to characterize binding events; surface biotinylation allows for fluorescent detection using DyLight 680-conjugated streptavidin, and fluorescence intensity is used to determine the extent of melanin binding for each peptide. FIG. 1B shows an initial classification model was trained using the data generated in FIG. 1A; random peptides are fed into the model and classified as melanin binding or non-melanin binding. FIG. 1C shows another microarray with 5,499 peptides, analyzed to further construct a regression super learner model (M. J. van der Laan, et al., Stat Appl Genet Mol Biol, 6:25, (2007)). FIG. 1D shows how conditional random peptides are generated based on position-dependent amino acid frequencies calculated using the peptide array data generated in FIG. 1C, with melanin binding levels of peptides further predicted by the regression model; peptides with desired melanin binding levels are selected for further experimental validation.

FIGS. 2A-2B are bar graphs showing synthesis and characterization of mNPs and biotinylated mNPs (b-mNP). FIG. 2A shows size (Number mean (d.nm, 0-250)) for each of mNPs and biotinylated mNPs (b-mNPs); FIG. 2B shows ζ-potential (0 to −30) of mNPs and b-mNPs (n=6), *p<0.05.

FIG. 3A is a schematic showing the in vitro melanin binding assay (mNPs) with Thermo Fisher fluorescence biotin quantitation kit; the DyLight 494-tagged avidin fluoresces when the weakly interacting HABA (H) is displaced by the biotinylated peptides; and FIG. 3B is a bar graph showing relative binding of brimonidine tartrate and sunitinib malate to mNPs (●) and biotinylated mNPs (□), respectively (n=3). No significant difference in relative binding was observed for either drug. Data shown as mean±SD.

FIGS. 4A-4B are show the results of a pilot 119 melanin binding peptide microarray screen. FIG. 4A is a graph of the interaction profiling of biotinylated mNPs against 16 positive control peptides and 103 random peptides, shown as sparklines, with data shown for peptide microarray screens performed at varying assay conditions (PBS at pH 6.5 or pH 7.4 or ultra-pure water with or without Tween 20 at 4 C or room temperature) at concentrations of 10 μg/mL (data not shown), 100 μg/mL, or 500 μg/mL, respectively; fluorescence intensities are plotted at the same scale. FIG. 4B is a permutation-based feature importance analysis of the melanin binding classification random forest (Breiman, Random forests. Mach Learn, 45(1), 5-32, (2001)), with the x-axis indicating the mean decrease in prediction accuracy after feature permutation, where the real value is also shown at the end of the bar; the top 20 important features are ranked by mean decrease in accuracy, showing synthesis and characterization of mNPs and biotinylated mNPs (b-mNP).

FIGS. 5A-5B are graphs showing data reduction within a random forest. FIG. 5A is a bar graph showing the original number of features (0-1,094) over 0-100 iterations; the vertical dashed line indicates the iteration where the number of features first reached 527; FIG. 5B is a bar graph Number of features remaining (1,100-500), and proportion of variance explained (coefficient of determination, R²) by the model ensemble after each iteration; the results of the first 100 iterations were noted as black dots, and those of another 100 iterations were indicated as gray circles. The vertical dashed line notes where the number of features is 527.

FIG. 6 is a flow-chart of the protocol, including holdout predictions, selection of optimal combination of base models, and final super learner ensemble.

FIG. 7 is a graph of coefficients of base models in the final melanin binding regression super learner ensemble, showing the top 20 base models with the highest coefficients. Coefficient values are noted at the end of the bar. Grayscale colors represent values of coefficient of determination (R²), which are also noted as white texts on each bar. Models were named according to the hyperparameter grid (e.g., g1) and order of model generated based on that grid (e.g., m179). Models without the grid number were generated based on pre-specified hyperparameters.

FIGS. 8A-8D are graphs of data reduction and base model coefficients in cell-penetration and toxicity final classification super learner ensembles, showing shows the number of features (701-1,094) over cell-penetration FIG. 8A and toxicity FIG. 8B, respectively. FIGS. 8C-8D are graphs indicating the top 20 base model coefficients in cell-penetration (FIG. 8C) and toxicity (FIG. 8D) final super learner ensembles, respectively. Values at the bar ends are coefficients. Base model performance in terms of accuracy is shown as grayscale colors and noted as white texts on the bars.

FIG. 9 is a graph showing comparison of super learner predictions to experimental melanin binding. Dots represent peptides. The x-axis shows melanin binding predictions from the super learner. The y-axis indicates converted experimental melanin binding values based on a simple log-linear regression model. Experimental melanin binding was obtained from the mNP assay (n=4).

FIGS. 10A-10F are graphs of the comparison of melanin binding and cell-penetration of peptides in validation experiments. Synthetic biotinylated peptides were incubated with ARPE-19 cells that were either induced or not induced to produce melanin (n=3). Dots represent peptides. FIGS. 10A-10D show intracellular peptide concentration in melanin-induced or non-induced cells over melanin binding measured from mNP assay (0-100%), for each of non-cell-penetration peptides non-CPP (FIGS. 10A-10B) and CPP (FIGS. 10C-10D), respectively. FIGS. 10E-10F show graphs of intracellular concentrations of CPP and non-CPP in induced (FIG. 10E) and non-induced cells (FIG. 10F), respectively. Data are shown as boxplot indicating median with 1^{st} and 3^{rd} quartiles, with regression lines noted as black lines, and 95% confidence intervals shown as shaded areas. The largest and smallest values within the 1.5 interquartile range are shown as the upper and lower whiskers; p-values were calculated with the Mann-Whitney test. ****p≤0.0001.

FIGS. 11A-11C are graphs of feature contributions to multifunctional peptide predictions, showing Shapley additive explanation (SHAP) analysis (Lundberg, et al., A unified approach to interpreting model predictions, Advances in Neural Information Processing Systems, volume 30, (2017)) of melanin binding regression (FIG. 11A), cell-penetration classification (FIG. 11B), and toxicity (FIG. 11C), classification predictions, respectively. The prediction outputs are on the scale of 0 to 100 for all regression and classification models. Dots displayed for each feature represent the peptides generated for experimental validation. Feature values are normalized based on the training data and are noted as grayscale colors. Top 20 features ranked by mean absolute SHAP value are shown. The black solid lines indicate HR97.

FIGS. 12A-12B are graphs of in vitro stability of HR97-brimonidine, showing In vitro stability of HR97-brimonidine conjugate in human aqueous for 28 days, at each time point (Day 1, 7, 14, 21 and 28), as quantified by HPLC (n=3) (FIG. 12A); and Cathepsin cleavage assay, showing % HR97-brimonidine conjugate (0-140%) for each of human cathepsin cocktails or control (buffer only) (n=3) (FIG. 12B). Data are presented as mean±SD, *p<0.01.

FIGS. 13A-13C are graphs of a single intracameral injection of HR97-brimonidine conjugate provides sustained reduction of IOP in pigmented rabbits, showing comparison of the IOP change from baseline (ΔIOP) for each of HR97-brimonidine conjugate (●), and brimonidine solution (□), respectively (FIG. 13A); and bar graph of cumulative ΔIOP was characterized by calculating the area under the curve over the 20 day measurement period (AUCₗₐₛₜ) for each of HR97-brimonidine conjugate (●), and brimonidine solution (□), respectively (FIG. 13B); and graph of comparison of the ΔIOP after a single topical dose of Alphagan P 0.1% (Δ), compared to ICM injection of brimonidine (□), and HR97-brimonidine conjugate (●) (FIG. 13C). All data presented as mean±SD (n=5 per group), *p<0.05.

FIGS. 14A-14D are graphs of HR97-sunitinib stability in human aqueous humor and increased solubility, showing HR97-sunitinib conjugate in human aqueous (%) over 0 to 28 days (n=3) (FIG. 14A); and in human vitreous for 28 days (n=3) (FIG. 14B); and bar graphs of cathepsin cleavage assay for % HR97-sunitinib conjugate for each of control and Cathepsin groups, respectively (n=3) (FIG. 14C); and solubility for each of sunitinib base, sunitinib malate and HR97-sunitinib, respectively (n=3) (FIG. 14D). Data presented as mean±SD, *p<0.05.

FIGS. 15A-15C are schematics of topical dosing of HR97-SunitiGel over at least 2 weeks in a rat model of retinal ganglion cell (RGC) loss. Rats received seven daily topical doses followed by varying durations of time of 7 (FIG. 15A); 14 (FIG. 15B); or 28 days (FIG. 15C); after the last dose before optic nerve head (ONH) crush was performed. The retinas were harvested 7 days after the ONH crush and the RGCs were quantified. FIGS. 15D-15E are bar graphs, showing quantification of RGCs Density (per mm²) for each of water (▴) and HR97-Sunitib gel for 1 week (□), 2 weeks (◯), or 3 weeks (●), respectively (FIG. 15D) Data presented as mean±SD (n=9-12 per group), *p<0.05; RGCs Density (per mm²) for each of water (▴) and Sunitib gel for 2 week (□), and HR97-Sunitib gel 2 weeks (◯), respectively (FIG. 15E). Data presented as mean SD (n=5-12 per group), *p<0.05.

FIGS. 16A-16D shows the synthesis scheme for HR97-sunitinib. In FIG. 16A, MC-Val-Cit-PAB-OH was suspended in DMF and activated with thionyl chloride (SOCl2) at 4° C. for 30 minutes. In FIG. 16B, the purified MCVal-Cit-PAB-Cl were then conjugated to sunitinib base in the presence of tetrabutylammonium iodide (TABI) and N,N-diisopropylethylamine in DMF at room temperature for 24 hours. In FIG. 16C, HR97 with a terminal cysteine was conjugated to MC-Val-Cit-PAB-sunitinib via the thiol-maleimide reaction in PBS solution. In FIG. 16D, the HR97-sunitinib was designed for release of intact parent drug when triggered by an intracellular chemical and enzymatic event, such as protease cleavage of the amide bond.

FIG. 17 shows the molecular structure of HR97-sunitinib. The m/z calculated for C₁₁₄H₁₈₀FN₃₇O₂₂S+ was 2,470.38, and the conjugate was measured at 2,511.11 [M−H+K]⁺.

FIGS. 18A and 18B are graphs showing the characterization of HR97-sunitinib melanin binding and cell uptake in vitro. FIG. 18A is a dot plot showing in vitro melanin binding capacity and dissociation constant of HR97-biotin, HR97-sunitinib, and sunitinib malate (n=3). Lower dissociation constant indicates stronger binding. Data are presented as mean±SD. FIG. 18B is a bar graph showing ARPE-19 cells cultured under normal conditions (ARPE) or under conditions that induce melanin production (induced ARPE) and incubated with sunitinib malate or HR97-sunitinib for 6 h. The cells were then collected and washed prior to extracting sunitinib. Drug content was normalized to per 1 million cells. Data are shown as mean±SD, n=3.

FIG. 19 is a line graph showing RGC density quantified by the Faster R-CNN Inception Resnet v2 cell counting program. RGC numbers were converted to numbers per mm² tissue area. Data are presented as mean±SD.

FIGS. 20A and 20B are bar graphs showing the characterization of intraocular drug concentrations after topical dosing with SunitiGel or HR97-SunitiGel in rats and rabbits. In FIG. 20A, Brown Norway rats were dosed unilaterally with SunitiGel or HR97-SunitiGel once daily for 7 days, and ocular tissues were collected 14 days after the last dose (consistent with the 2-week dosing regimen shown in FIG. 15A-15C). Combined levels of sunitinib and N-desethyl sunitinib were reported per tissue sample. Data are presented as mean±SEM (n=6 per group). In FIG. 20B, Dutch Belted rabbits were dosed unilaterally with SunitiGel or HR97-SunitiGel once daily for 7 days, and ocular tissues were collected 2 hours after the last dose. Combined levels of sunitinib and N-desethyl sunitinib were reported per tissue sample. Data are presented as mean±SEM (n=4 per group).

### DETAILED DESCRIPTION OF THE INVENTION

### I. Definitions

The terms “gel” and “hydrogel”, as used herein, refers to a swollen, water-containing network of finely dispersed polymer chains that are water-insoluble, where the polymeric molecules are in the external or dispersion phase and water (or an aqueous solution) forms the internal or dispersed phase. The chains can be chemically crosslinked (chemical gels) or physically crosslinked (physical gels). Chemical gels possess polymer chains that are connected through covalent bonds, whereas physical gels have polymer chains linked by non-covalent bonds or cohesion forces, such as Van der Waals interactions, ionic interaction, hydrogen bonding, or hydrophobic interaction.

The polymer chains are typically hydrophilic or contain hydrophilic polymer blocks. “Gel-forming polymers” is used to describe any biocompatible polymer, including homopolymers, copolymers, and combinations thereof, capable of forming a physical hydrogel in an aqueous medium when present at or above the critical gel concentration (CGC).

The “critical gel concentration”, or “CGC”, as used herein, refers to the minimum concentration of gel-forming polymer needed for gel formation, e.g., at which a solution-to-gel (sol-gel) transition occurs. The critical gel concentration can be dependent on a number of factors, including the specific polymer composition, molecular weight, temperature, and/or the presence of other polymers or excipients.

The term “thermosensitive gel-forming polymer” refers to a gel-forming polymer that exhibits one or more property changes with a change in the temperature. For example, some thermosensitive gel-forming polymers are water soluble below a certain temperature but become water insoluble as temperature is increased. The term “lower critical solution temperature (LCST)” refers to a temperature, below which a gel-forming polymer and solvent are completely miscible and form a single phase. For example, “the LCST of a polymer solution” means that the polymer is uniformly dispersed in a solution at that temperature (i.e., LCST) or lower, but aggregates and forms a second phase when the solution temperature is increased beyond the LCST.

“Hydrophilic,” as used herein, refers to molecules which have a greater affinity for, and thus solubility in, water as compared to organic solvents. The hydrophilicity of a compound can be quantified by measuring its partition coefficient between water (or a buffered aqueous solution) and a water-immiscible organic solvent, such as octanol, ethyl acetate, methylene chloride, or methyl tert-butyl ether. If after equilibration a greater concentration of the compound is present in the water than in the organic solvent, then the compound is considered hydrophilic.

“Hydrophobic,” as used herein, refers to molecules which have a greater affinity for, and thus solubility in, organic solvents as compared to water. The hydrophobicity of a compound can be quantified by measuring its partition coefficient between water (or a buffered aqueous solution) and a water-immiscible organic solvent, such as octanol, ethyl acetate, methylene chloride, or methyl tert-butyl ether. If after equilibration a greater concentration of the compound is present in the organic solvent than in the water, then the compound is considered hydrophobic.

As used herein, the term “treating” includes inhibiting, alleviating, preventing, or eliminating one or more symptoms or side effects associated with the disease, condition, or disorder being treated.

The term “reduce”, “inhibit”, “alleviate” or “decrease” are used relative to a control, either no other treatment or treatment with a known degree of efficacy. One of skill in the art would readily identify the appropriate control to use for each experiment. For example, a decreased response in a subject or cell treated with a compound is compared to a response in subject or cell that is not treated with the compound.

As used herein the term “effective amount” or “therapeutically effective amount” means a dosage sufficient to treat, inhibit, or alleviate one or more symptoms of a disease state being treated or to otherwise provide a desired pharmacologic and/or physiologic effect. The precise dosage will vary according to a variety of factors such as subject-dependent variables (e.g., age, immune system health), the disease or disorder, and the treatment being administered. The effective amount can be relative to a control. Such controls are known in the art and discussed herein, and can be, for example the condition of the subject prior to or in the absence of administration of the drug, or drug combination, or in the case of drug combinations, the effect of the combination can be compared to the effect of administration of only one of the drugs.

As generally used herein “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

“Biocompatible” and “biologically compatible”, as used herein, generally refer to materials that are, along with any metabolites or degradation products thereof, generally non-toxic to the recipient, and do not cause any significant adverse effects to the recipient. Generally speaking, biocompatible materials are materials which do not elicit a significant inflammatory, immune or toxic response when administered to an individual.

“Excipient” is used herein to include a compound that is not a therapeutically or biologically active compound. As such, an excipient should be pharmaceutically or biologically acceptable or relevant, for example, an excipient should generally be non-toxic to the subject. “Excipient” includes a single such compound and is also intended to include a plurality of compounds.

The term “osmolarity”, as generally used herein, refers to the total number of dissolved components per liter. Osmolarity is similar to molarity but includes the total number of moles of dissolved species in solution. An osmolarity of 1 Osm/L means there is 1 mole of dissolved components per L of solution. Some solutes, such as ionic solutes that dissociate in solution, will contribute more than 1 mole of dissolved components per mole of solute in the solution. For example, NaCl dissociates into Na⁺ and Cl⁻ in solution and thus provides 2 moles of dissolved components per 1 mole of dissolved NaCl in solution. Physiological osmolarity is typically in the range of about 280 to about 310 mOsm/L.

The term “tonicity”, as generally used herein, refers to the osmotic pressure gradient resulting from the separation of two solutions by a semi-permeable membrane. In particular, tonicity is used to describe the osmotic pressure created across a cell membrane when a cell is exposed to an external solution. Solutes that can cross the cellular membrane do not contribute to the final osmotic pressure gradient. Only those dissolved species that do not cross the cell membrane will contribute to osmotic pressure differences and thus tonicity. The term “hypertonic”, as generally used herein, refers to a solution with a higher concentration of solutes than is present on the inside of the cell. When a cell is immersed into a hypertonic solution, the tendency is for water to flow out of the cell in order to balance the concentration of the solutes. The term “hypotonic”, as generally used herein, refers to a solution with a lower concentration of solutes than is present on the inside of the cell. When a cell is immersed into a hypotonic solution, water flows into the cell in order to balance the concentration of the solutes. The term “isotonic”, as generally used herein, refers to a solution wherein the osmotic pressure gradient across the cell membrane is essentially balanced. An isotonic formulation is one which has essentially the same osmotic pressure as human blood. Isotonic formulations will generally have an osmotic pressure from about 250 to 350 mOsm.

“Gel-forming concentration” refers to the concentration of the polymer at which it undergoes a phase shift from a solution to a gel. Critical gelation concentration (CGC) corresponds to the minimum concentration required for gelation.

### II. Compositions

Peptides or conjugates thereof having these multiple functionalities are described.

### A. Peptides Having Multiple Functionalities

Peptides having multiple functionalities, for example, including: (i) high binding to a cellular target (e.g., melanin pigment in the eye); (ii) high cell-penetration (e.g., to enter cells and access melanin in the melanosomes); and (ii) low cytotoxicity are described.

In one embodiment, the peptide having multiple functionalities including (i) high binding to a cellular target (e.g., melanin pigment in the eye), (ii) high cell-penetration (e.g., to enter cells and access melanin in the melanosomes), and (ii) low cytotoxicity, is HR97 (FSGKRRKRKPR, SEQ ID NO. 1).

In some embodiments, the peptide includes HR97 variants having about 50% and 95%, inclusive; about 55% and about 90%, inclusive; about 60% and about 85%, inclusive; about 70% and about 80%, inclusive; identical to the amino acid sequence of SEQ ID NO:1. For example, if 10 out of the 11 amino acids of HR97 remain the same, the identify is about 90.9%, and if 6 out of the 11 amino acids of HR97 remain the same, the identify is about 54.5%.

In some embodiments, the peptide has the following regular expression:

F{1,2}G|S{2}(K|R{0.5-1.0}|X){4,20}
where F is phenylalanine, G is glycine, S is serine, K is lysine, R is arginine, and X is any amino acid other than lysine or arginine,
where the vertical bar, “|”, is used to convey that either amino acid meets the pattern. So that GIS{2} represents the following: GG; GS; SG; SS. Integers in braces, { } represent the number of times the preceding amino acid(s) might be represented, e.g., {1,2} means once or twice. Braces are also used to represent proportions when represented when used with a floating point number or range <1.0. Parentheses are used to group elements, used once here.

In prose, the regular expression can be interpreted as follows—1 or 2 phenylalanine, any combination of 2 glycine and/or serine (i.e., GG; GS; SG; or SS), any the collective combination of 4 to 20 amino acids having lysine and/or arginine at a proportion of 0.5 to 1.0, i.e., 50% to 100% of the 4 to 20 amino acids are lysine and/or arginine and the remainder can be any other amino acids.

A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by a genetic code. A variant of a polypeptide may be naturally occurring such as an allelic variant, or it may be a variant that is not known to occur naturally.

Modifications and changes can be made in the structure of the polypeptides disclosed herein and still have similar characteristics to the original polypeptide (e.g., a conservative amino acid substitution). For example, certain amino acids can be substituted for other amino acids in a sequence without appreciable loss of activity. Since it is the interactive capacity and nature of a polypeptide that defines biological functional activity, certain amino acid sequence substitutions can be made in a polypeptide sequence and nevertheless obtain a polypeptide with like properties.

Amino acid substitutions are generally based on the relative similarity of the amino acid side-chain substituents, for example, their hydrophobicity, hydrophilicity, charge, and size. Exemplary substitutions that take various of the foregoing characteristics into consideration are well known to those of skill in the art and include (original residue: exemplary substitution(s)): (Ala: Gly, Ser), (Arg: Lys), (Asn: Gln, His), (Asp: Glu, Cys, Ser), (Gin: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Trp: Tyr), (Tyr: Trp, Phe), and (Val: Ile, Leu). The polypeptides can include variants having about or more than 50%, 60%, 70%, 80%, 90%, and 95% sequence identity to the polypeptide of interest.

“Identity” and “similarity” can be readily calculated by known methods, such as those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo and Lipman, SIAM J Applied Math, 48: 1073 (1988)).

Preferred methods to determine identity are designed to give the largest match between the sequences tested. Methods to determine identity and similarity are codified in publicly available computer programs. The percent identity between two sequences can be determined by using analysis software (i.e., Sequence Analysis Software Package of the Genetics Computer Group, Madison Wis.). The default parameters are used to determine the identity for the polypeptides of the present disclosure.

### B. Conjugates of Peptides and Active Agents

Typically, the peptides having multiple functionalities are conjugated to one or more agents to be delivered to the eye. An exemplary conjugation of HR97 to the quaternary-ammonium-linked brimonidine via a thiol-maleimide reaction is shown in Formula I. An exemplary conjugation of HR97 to the quaternary-ammonium-linked sunitinib via a thiol-maleimide reaction is shown in II.

In some embodiments, the conjugation of an active agent to a peptide having multiple functionalities is very stable in water/saline or physiological conditions prior to proteolytic cleavage inside cells, for example, less than 20%, less than 15%, less than 10%, less than 5% of the conjugated molecules have the active agent cleaved within a week, two weeks, three week, four weeks, a month, two months, three months, four months, five months, or six months.

### 1. Therapeutic, Prophylactic, and Diagnostic Agents

Examples include therapeutic agents, prophylactic agents, and/or diagnostic agents. A biologically active agent is a substance used for the treatment (e.g., therapeutic agent), prevention (e.g., prophylactic agent), diagnosis (e.g., diagnostic agent), or to effect a cure or mitigation of disease or illness, alter the structure or function of the body, or pro-drugs, which become biologically active or more active after they have been placed in a predetermined physiological environment. These may be small-molecule drugs ((e.g., molecular weight less than 2000, 1500, 1000, 750, or 500 atomic mass units (amu)), peptides or proteins, sugars or polysaccharides, nucleotides, or oligonucleotides such as aptamers, siRNA, and miRNA, lipids, glycoproteins, lipoproteins, or combinations thereof.

The agents can include one or more of those described in Martindale: The Complete Drug Reference, 37^{th} Ed. (Pharmaceutical Press, London, 2011).

In one embodiment, the agent to be delivered is poorly soluble in water, but soluble after conjugation to the peptide. In other embodiments, the agents are water-soluble.

i. Therapeutic and Prophylactic Agents

In some embodiments, the peptides having multiple functionalities are conjugated to one or more therapeutic agents. Therapeutic agents to be delivered can include anti-infective (antibiotics, antivirals, antifungals), agents for treatment of eye disorders (glaucoma, dry eye), anti-inflammatories, inhibitors of neovascularization and/or fibrosis, neuroactive agents, or chemotherapeutics for treatment of a disease such as aberrant neovascularization or cancer. Exemplary agents include sunitinib, brimonidine, brinzolamide, cyclosporine A, moxifloxacin, budesonide, and acriflavine.

In some embodiments, the formulations include one or more short therapeutic peptides. Examples of useful proteins include hormones such as insulin, growth hormones including somatomedins, and reproductive hormones. Examples of other useful drugs include neurotransmitters such as L-DOPA, antihypertensives or saluretics such as Metolazone from Searle Pharmaceuticals, carbonic anhydrase inhibitors such as Acetazolamide from Lederle Pharmaceuticals, insulin like drugs such as glyburide, a blood glucose lowering drug of the sulfonylurea class, synthetic hormones such as Android F from Brown Pharmaceuticals and TESTRED® (methyltestosterone) from ICN Pharmaceuticals.

Representative anti-proliferative agents include angiogenesis inhibitors, anti-VEGF compounds; receptor tyrosine kinase (RTK) inhibitors such as sunitinib (SUTENT®); tyrosine kinase inhibitors such as sorafenib (NEXAVAR®), erlotinib (TARCEVA®), pazopanib, axitinib, and lapatinib; transforming growth factor-α or transforming growth factor-β inhibitors. In one embodiment, the active agent is sunitinib. In another embodiment, the active agent is brimonidine.

In some embodiments, the therapeutic agent is an agent for treatment or prevention of a retinal disease, such as a degenerative retinal disease. Exemplary drug types include antioxidant molecules that scavenge and prevent oxidative and nitrosative damage, anti-infectives, corticosteroids, analgesics, nutraceuticals. In a preferred embodiment the agent is a small molecule drug for treating macular degeneration. Exemplary drugs include xanthophylls (Lutein), verteporfin (Visudyne), natamycin (Natacyn), sulfacetamide ophthalmic (Bleph-10), pegaptanib (Macugen), cephalosporin (ceftriaxone), and corticotropin.

ii. Diagnostic Agents

In some embodiments, the peptides have multiple functionalities are conjugated to diagnostic agents. These agents can also be used prophylactically. Examples of diagnostic agents include paramagnetic molecules, fluorescent compounds, magnetic molecules, and radionuclides, x-ray imaging agents, and contrast media. Examples of other suitable contrast agents include gases or gas emitting compounds, which are radioopaque.

Exemplary diagnostic agents include dyes such as fluorescent dyes and near infra-red dyes, SPECT imaging agents, PET imaging agents and radioisotopes.

### C. Hypotonic Gel-Forming Formulations

In some embodiments, the peptide-drug conjugates are formulated in a hypotonic gel-forming composition including one or more hydrogel forming polymers, preferably comprising poloxamers for enhanced delivery of therapeutic, diagnostic, prophylactic, or other agents, to epithelial tissues, especially those having a mucosal coating. The formulations are particularly suited for enhanced delivery of therapeutic, diagnostic, prophylactic, or other agents to the eye. In preferred embodiments, the epithelial tissues or surfaces are ocular surfaces.

The polymers are administered at a concentration less than their normal critical gelling concentration (CGC). A poloxamer gel administered onto the surface of a mucosal surface such as the eye at a concentration equal to or above its CGC will assemble into a bolus on the surface. On the ocular surface, a bolus of gel is rapidly cleared away by blinking. In contrast, fluid from a hypotonically-administered poloxamer solution, where the poloxamer is at a concentration below its CGC, will form a uniform, thin gel coat across the entire surface of the eye, thereby maintaining a reservoir of drug in close association with the mucosal surface and enhancing and facilitating delivery of agents to the eye. As water is absorbed into the tissues, the poloxamer becomes concentrated and forms a gel near the epithelial tissue surface, thereby trapping drug molecules in a sustained-release gel on the tissue surface (rather than, e.g., in a bolus of gel that forms primarily in the lumen as occurs with traditional thermogelling methods whereby the gelling polymers are administered at a concentration at or above their CGC).

Typically, the hydrogel including the peptide-drug conjugates forms a thin coating that covers the entire ocular surface. The size and thickness of the coating is dependent upon the amount of the material applied and the size/shape of the ocular surface. In some embodiments the hydrogel forms a thin gel at the surface of the eye that is between about 0.01 mm and about 2 mm in thickness, inclusive, or between about 0.5 mm and 1.5 mm, inclusive; for example, about 0.01 mm, 0.05, 0.1, 0.15, 0.2, 0.3. 0.4. 0.5, 0.6. 0.7, 0., 0.9, 1.0, 1.5, or 2 mm in thickness, or more than 2 mm in thickness. In some embodiments, the gel forms a film that coats the entire ocular surface with uniform thickness. In other embodiments, the gel coats the ocular surface with a film that is non-uniform in thickness, for example, having a concave or convex conformation. In some embodiments, the gel film at the ocular surface prevents pathogens from contacting the surface of the eye. For example, in some embodiments, the gel film prevents bacteria, viruses, fungi or protozoan pathogens from contacting the eye.

### 1. Hydrogel-Forming Polymers

The hypotonic gel-forming compositions contain one or more gel-forming polymers. Gel-forming polymers are utilized at a concentration below the normal critical gel concentration (CGC) of the polymer, e.g., the concentration at which the polymer solution would gel in a test tube when warmed to 37 C.

Thermosensitive or thermoresponsive hydrogels form solutions that undergo sol-gel transitions when the following criteria are met:
1) at or above the critical gelling concentration (CGC), and
2) at or above the critical gelling temperature.

Thermosensitive gelling agents (at or above their CGC) used for biomedical applications are liquid at room temperature but form a gel at body temperature. The increase in temperature induces a rearrangement and alignment of the polymer chains, leading to gelation into a three-dimensional structure. This phenomenon is generally governed by the ratio of hydrophilic to hydrophobic moieties on the polymer chain. A common characteristic is the presence of a hydrophobic methyl, ethyl, or propyl group.

Thermosensitive polymers that fit these criteria can be administered topically in a hypotonic solution at a range of concentrations that is below its CGC to mucosal and/or epithelial tissues to form a uniform gel coating in vivo.

Examples of thermosensitive gel forming polymers that can be used include polyoxyethylene-polyoxypropylene-polyoxyethylene triblock copolymers such as, but not limited to, those designated by the CTFA names poloxamer 407 (CAS 9003-11-6, molecular weight 9,840-14,600 g/mol, percentage of polyoxyethylene by weight approximately 70%; available from BASF as LUTROL® F127) and poloxamer 188 (CAS 9003-11-6, molecular weight 7,680-9,510 g/mol, percentage of polyoxyethylene by weight approximately 80%; available from BASF as LUTROL® F68); Poloxamers are also known by the trade name PLURONIC® e.g., PLURONIC® F98 (CAS 9003-11-6, molecular weight 13 kg/mol, percentage of polyoxyethylene by weight approximately 80%; available from BASF); Tetronics tetra-functional block copolymers based on ethylene oxide and propylene oxide available from BASF as TETRONIC®; poly(N,N-diethylacrylamide); poly(N,N-dimethylacrylamide); poly(N-vinylcaprolactam); poly(N-alkylacrylamide); poly(N-vinylalkylamide); poly(N-isopropyl acrylamide); polyethylene oxide methacrylate polymers; poly(lactic-co-glycolic acid) (PLGA)-polyethylene glycol triblock copolymers (PLGA-PEG-PLGA and PEG-PLGA-PEG); polycaprolactone (PCL)-polyethylene glycol triblock copolymers (PCL-PEG-PCL and PEG-PCL-PEG); chitosan; and combinations thereof.

The hydrogels can be formed from individual gel forming polymers or as a combination of gel formers. For example, a poloxamer and another gel forming polymer (e.g., a tetronic polymer) may be used in combination to attain the desired characteristics. In addition, various forms of the same gel former (e.g., Poloxamer 188 and Poloxamer 407) can be combined to attain the desired characteristics.

The polymer is provided in a concentration less than the concentration in aqueous solution that forms a gel in a test tube when heated to 37 C. The concentration must be sufficiently high, but below the CGC, for the epithelium to absorb enough fluid for the CGC to be reached in vivo, so gelation can occur preferentially on/near the mucosal epithelial surface, for example, at the surface of the eye. The range of time that it takes for gelation to occur depends on the mucosal surface (the capacity and rate of water absorption), the tonicity of the solution administered (more hypotonic solutions will drive more rapid fluid absorption), and the concentration of polymer administered (if the polymer concentration is too low, not enough fluid absorption will occur to concentrate the polymer to its CGC). Gelation generally occurs within 1-20 seconds, inclusive, upon administration onto the surface of the eye, for example, as an eyedrop. For example, in some embodiments, gelation occurs within less than a second, or within 1-2 seconds, 2-3, 3-4, 4-5, 5-6, 6-7, 7-8, 8-9, 9-10, or less than 5 seconds, or less than 10 seconds following administration onto the surface of the eye.

Therefore, in some embodiments, the gel forming polymer is present within the formulation in an amount between 1% and 50%, for example, between 5% and 20%, inclusive, such as 5%, 6%, 7%, 8%, 9%, 10%, 11%, 12%, 13%, 14%, 15%, 16%, 17%, 18%, 19% or 20%. Typically, formulations for administration to the ocular surface include an amount of hydrogel forming polymer that is a concentration below the critical gel concentration (CGC) of the polymer under isotonic conditions and a temperature between room temperature and body temperature (from about 25 C to about 37 C, inclusive).

A preferred hydrogel forming polymer is poly(ethylene glycol)-block-poly(propylene glycol)-block-poly(ethylene glycol) (PLURONIC® F127; “F127”). In some embodiments, formulations for administration to the eye include PLURONIC® F127 at a concentration below the CGC, for example, between 5% and 18%, inclusive, for example, between about 8% and about 15%, between about 10% and about 14%, or between about 11% and about 13%, inclusive. In a particular embodiment, the PLURONIC® F127 is present in an amount of approximately 12%.

### 2. Hypotonic Carriers

The gel-forming compositions include a hypotonic carrier. The hypotonic carrier will typically be a biocompatible carrier that preferably causes little to no signs of irritation when administered to the eyes of human subjects. The carrier can be naturally occurring or non-naturally occurring including both synthetic and semi-synthetic carriers. Preferred carriers are water-based. Other solutions, including sugar-based (e.g., glucose, mannitol) solutions and various buffers (phosphate-buffers, tris-buffers, HEPES), may also be used.

When hypotonic solutions are applied to an epithelial surface, such as the surface of the eye, a fluid shift occurs, and water is moved into the epithelial tissue. This can cause swelling of the epithelial cells. In some cases, when the osmotic pressure difference is too large, the epithelial cells may burst causing tissue irritation or disruption of the epithelial membrane.

“Hypotonic” solution refers to a solution that causes water absorption by the epithelial surface to which it is administered. Examples of hypotonic solutions include, but are not limited to, Tris[hydroxylmethyl]-aminomethane hydrochloride (Tris HCl, 10-100 mM, pH. 6-8), (4-(2-hydroxyethyl)-1-piperazineethanesulfonic acid (HEPES, 10-100 mM, pH 6-8) and dilute solutions of PBS, such as a solution containing 0.2 g KCl, 0.2 g KH₂PO₄, 8 g NaCl, and 2.16 g Na₂HPO₄*7H₂O in 1000 ml H₂O.

Hypotonic carriers cause dissolved gel-forming polymers to concentrate at an epithelial surface, resulting in uniform gel formation on the surface. The hypotonic carrier usually contains water as the major component. The hypotonic carrier can be water, although mixtures of water and a water-miscible organic solvent can also be used. Suitable water-miscible organic solvents include alcohols, such as ethanol, isopropanol; ketones, such as acetone; ethers such as dioxane; and esters such as ethyl acetate.

Therefore, in some embodiments, the hypotonic carrier includes one or more osmolarity modifying excipients. Sodium chloride is the excipient that is most frequently used to adjust osmolarity if a solution is hypotonic. Other excipients used to adjust hypotonic solutions include glucose, mannitol, glycerol, propylene glycol and sodium sulfate. Osmolarity modifying excipients can include pharmaceutically acceptable salts such as sodium chloride, sodium sulfate, potassium chloride, and other salts to make buffers such as dibasic sodium phosphate, monobasic potassium phosphate, calcium chloride, and magnesium sulfate. Other excipients used to adjust tonicity can include glucose, mannitol, glycerol, or propylene glycol.

In some embodiments, the hypotonic carrier has any osmolarity less than the effective isotonic point (the concentration at which fluid is neither absorbed nor secreted by the underlying tissues) at that mucosal surface. The isotonic point varies for different mucosal surfaces and different buffers, depending on active ion transport at that epithelial surface, e.g., 0.9% solution of NaCl (Normal Saline) is iso-osmotic with blood and tears. Human tear fluid has an osmolality in the range of 280-320 mOsm/L, while the osmolarity of the aqueous layer of the precorneal tear film is approximately 300 mOsm/L. Therefore, in some embodiments the solution has a tonicity at or below 280 mOsm/L, for example, from 50 mOsm/L to 280 mOsm/L, from 100 mOsm/L to 280 mOsm/L, from 150 mOsm/L to 250 mOsm/L, from 200 mOsm/L to 250 mOsm/L, from 220 mOsm/L to 250 mOsm/L, from 220 mOsm/L to 260 mOsm/L, from 220 mOsm/L to 270 mOsm/L, or from 220 mOsm/L to 280 mOsm/L.

The isotonic point in the vagina for sodium-based solutions is about 300 mOsm/L, but in the colorectum, it is about 450 mOsm/L. In some embodiments the solution is hypotonic at the mucosal surface of colorectum, having a tonicity at or below 400 mOsm/L, for example, from 50 mOsm/L to 400 mOsm/L; at or below 350 mOsm/L, for example, from 50 mOsm/L to 350 mOsm/L; or at or below 300 mOsm/L, for example, from 50 mOsm/L to 280 mOsm/L. In some embodiments, the solution is hypotonic at the mucosal surface of vagina, having a tonicity at or below 300 mOsm/L, for example, from 50 mOsm/L to 280 mOsm/L.

The hypotonic carrier can include one or more pharmaceutically acceptable acid, one or more pharmaceutically acceptable base, or salts thereof. Pharmaceutically acceptable acids include hydrobromic, hydrochloric, and sulphuric acids, and organic acids, such as methanesulphonic acids, tartaric acids, and malic acids. Pharmaceutically acceptable bases include alkali metal (e.g., sodium or potassium) and alkali earth metal (e.g., calcium or magnesium) hydroxides and organic bases such as pharmaceutically acceptable amines. The hypotonic carrier can include pharmaceutically acceptable buffers such as citrate buffers or phosphate buffers.

In some embodiments, the formulations can be prepared as liquids for administration onto the surface of the eye. The gel forming liquid or polymer solubilizes insoluble drugs by forming micelles. Powder can be made by freeze-drying and reconstituted at the time of use.

The formulations may also include pharmaceutically acceptable diluents, preservatives, solubilizers, stabilizer, emulsifiers, adjuvants and/or carriers. Stabilizers such as SPAN® 20 (sorbitan laurate, CAS Number 1338-39-2) may facilitate dissolution and prevent re-aggregation. Other exemplary stabilizers include polysorbates or TWEENS®, e.g., poly-sorbate 20, polysorbate 60, polysorbate 65 and polysorbate 80, and polyglycerol esters (PGE), polyoxyethylene alkyl ethers, poloxyl stearates, fatty acids (e.g., oleic acid) and propylene glycol monostearate (PGMS). In some cases, the composition includes one or more stabilizers.

Dosage formulations will typically be prepared as single or multiple liquid or dry dosage units in an appropriate applicator, for example, an eye drop dispenser. A person of ordinary skill in the art will be aware of many options for drug storage and application, such as dual chambered devices that may be used to keep various components separate during storage. Multiple dosage units will typically include a barrel loaded with powder, and a plunger having dosage increments thereon. These will typically be sterilized and packaged in sealed, sterile packaging for storage and distribution. See also Remington: The Science and Practice of Pharmacy, 22nd Edition.

Dosage unit administrators may be designed to fit the anatomic location to which drug is to be delivered, such as intraocular administration by one or more eye drops. In an exemplary embodiment, the formulation is a solution having a total volume of between about 0.01 ml and about 2.5 ml, inclusive, for extrusion of drops each of between about 10 μl and about 200 μl, inclusive, e.g., 50 μl/drop).

### III. Methods of Screening for Peptides Having Multiple Desired Properties

In some embodiments, screening for the cell-penetrating peptides includes: (1) generating pools of potential cell-penetrating peptides with the AI methodology that adapt the Super Learning (SL) approach and trained based on the cell-penetrating peptide dataset (Agrawal, P. et al., CPPsite 2.0: a repository of experimentally validated cell-penetrating peptides. Nucleic Acids Res 44, D1098-1103 (2016)); (2) incubating the peptide pools with cell lines of interest, including the corneal epithelial cell, corneal endothelial cell, non-pigmented ciliary epithelium, pigmented ciliary epithelium, retina pigment epithelial cell, rod cell, cone cell, retinal ganglion cell, Muller/glial cell, skin melanocyte, skin keratinocyte, Markel cell; and (3) extracting the intracellular peptide and analyze the peptide concentration with HPLC or LC-MS system to identify the top cell-penetrating peptides.

Systems and methods of experimental screening, analyzing, and computational modeling to engineer multifunctional peptides that can be chemically conjugated to drugs to enhance the pharmacokinetics and pharmacodynamics upon administration to the body (e.g., the eye) has been established. In some embodiments, an artificial intelligence (AI) methodology that adapts the Super Learning (SL) approach to engineer multifunctional peptides is used. In some embodiments, the systems implement Artificial Intelligence (AI) to allow for designing peptide sequences that simultaneously provide multiple desired properties, including high binding to melanin (pigment in the eye), high cell-penetration (to enter cells and access melanin in the melanosomes), and low cytotoxicity.

Methods of screening for peptides having multiple desired functional properties typically are used to identify a plurality of polypeptides having amino acid sequences that selectively bind a specific cellular target (e.g., cellular proteins), and then select variants and subsets of the selected polypeptide sequences that have at least one additional desired function. In some embodiments, the subset or variant of the polypeptide has the function of cell penetration, in addition to the selective binding to the cellular target. Typically, the screening also involves one or more subsequent screening steps to further select the polypeptides based on a third property, such as cytotoxicity. In some embodiments, target binding is the first screen, cell penetration the second screen, and toxicity the third screen.

Therefore, methods for selecting a polypeptide based on an input sequence include the steps: (i) screening for selective binding to a desired cellular target to identify one or more target binding polypeptides; (ii) generating a plurality of variants based on the target binding polypeptides identified in (i) to create a first target-binding polypeptide library; (iii) screening the first target-binding polypeptide library to identify a subset of target binding polypeptides; (iv) screening the subset of target binding polypeptides for cell-penetration and selecting a subset of cell-penetrating, target binding polypeptides; (v) screening the subset of cell-penetrating, target binding polypeptides for toxicity to identify a subset of non-toxic, cell-penetrating target binding polypeptides.

In some embodiments, screening for one or more target binding polypeptides is implemented using an in vitro binding assay, for example, an enzyme-linked immunoassay (ELISA), a phage display library, or a microarray system. In preferred embodiments, to generate sufficient data for use in training a machine learning model, a high-throughput flow-based microarray system is used to screen peptides for target binding. In some embodiments, when the high-throughput flow-based microarray system is used, the peptide variants or candidates for screening for binding are immobilized on a solid surface such as microarray slides, and the target molecules are formulated to nanoparticles suitable for binding, i.e., with the binding surface exposed. In one embodiment, the cellular target is melanin. In another embodiment, melanin is processed into nanoparticles (mNPs) with an average size of about 200 nm and ζ-potential of between about −25 mV and −20 mV. In a further embodiment, the melanin nanoparticles are biotinylated for easy of subsequent detection once bound to one or more peptide variants immobilized on the microarray surface.

In some embodiments, each step includes one or more additional steps for creating a multiplicity of amino acids variations at each position within the target binding polypeptide(s) and repeating each of the screens in steps (i)-(v) to assess the target binding, cell penetration, and toxicity for each of the variant polypeptides. In some embodiments, peptide variants are generated with a frequency of 1-10%, 2-8%, 3-7%, or preferably 5% for each of the 20 amino acids based on the target binding polypeptides identified in (i). In some embodiments, peptides are between 4 and 20 amino acids, inclusive, between 5 and 15 amino acids, inclusive, between 7 and 12 amino acids, inclusive.

In some embodiments, the production of sequence variation is implemented by a computer. In some embodiments, the computer employs an algorithm to determine appropriate sequence variations. An exemplary algorithm is a tree-based statistical model implemented by a computer configured to identify polypeptide sequence variations having a likelihood of resulting in one or more or enhanced target binding, enhanced cellular penetration or reduced cytotoxicity as compared to an input polypeptide sequence.

In some embodiments, methods for screening for peptides having (i) high binding to melanin (pigment in the eye), (ii) high cell-penetration (to enter cells and access melanin in the melanosomes), and (ii) low cytotoxicity are described. In some embodiments, methods include the steps of (i) screening for selective binding to melanin to identify one or more target binding polypeptides; (ii) generating a plurality of variants based on the melanin binding polypeptides identified in (i) to create a first melanin-binding polypeptide library; (iii) screening the first melanin-binding polypeptide library to identify a subset of melanin binding polypeptides; (iv) screening the subset of melanin binding polypeptides for cell-penetration and selecting a subset of cell-penetrating, melanin binding polypeptides; (v) screening the subset of cell-penetrating, melanin binding polypeptides for toxicity to identify a subset of non-toxic, cell-penetrating melanin binding polypeptides.

In some embodiments, methods of building a classification or regression model capable of distinguishing peptides that bind to a cellular target from a plurality of peptides include one or more of the following steps:
a) assaying a plurality of peptides using one or more assays to provide a classifier or regressor based on the binding or non-binding towards the cellular target representative of each of the plurality of peptides;
b) identifying a set of features corresponding to properties of each of the plurality of peptides to be input to a machine learning model;
c) loading, into a memory of a computer system,
   the machine learning model comprising building a classification model based on the classifier from a), and the set of features corresponding to properties of each of the plurality of peptides, optionally obtaining an output classification.

In some embodiments, the methods also include one or more of the following steps:
d) generating a new library of peptide variants based on peptides that are identified as binders, by substituting one or more amino acids;
e) assaying the new library of peptides using one or more assays to provide a classifier based on the binding towards the cellular target representative of each peptide of the new library; and
f) identifying the same set of features corresponding to properties of each peptide of the new library;
g) inputting the classifier from e), and the set of features corresponding to properties of each peptide of the new library into the machine learning model of step c) to obtain an improve output classification; and
h) inputting a candidate peptide sequence into the output classification based on the machine learning model of step c) or g), and obtaining an output to indicate if the candidate peptide binds to the cellular target.

In some embodiments, the classification model is a random forest classification model. In some embodiments, the set of features corresponding to properties are physiochemical properties of each amino acids of each peptide.

### IV. Methods of Conjugating One or More Active Agents to Peptides with Multiple Functionalities

Methods of conjugating one or more active agents to peptides with multiple functionalities are also provided. In some embodiments, the methods use one or more linkers to conjugate to an active agent to the peptide. In preferred embodiments, conjugating one or more active agents to peptides with multiple functionalities minimally reduces the functionalities of the peptides, for example, the target binding and cell penetration is reduced less than 30%, less than 25%, less than 20%, less than 15%, less than 10%, less than 5% compared to an unconjugated peptide. In one embodiment, the peptide is conjugated to an active agent via a quaternary-ammonium traceless linker system.

Conjugation strategies for peptide drug conjugates include the hydrazine, ester, amide, disulfide, dipeptide (Val-Cit, Ala-Leu), tripeptide (Al), PEG-spaced short peptide-sequences (PEGn—Val-Cit-PAB-OH), beta-glucuronide and traceless quaternary ammonium system as the linker, depends on the target drug/vehicle that is in interests. In some cases, multi-arm linker systems include 2/4/8/9 arm PEG (1-20 kDa) with acid/acrylamide, aldehyde, Alkyne, amide, azide, DBCO, GA, GAA, hydrazide, maleimide, hydroxyl, SA, thiol modification could be used as peptide-drug conjugation linkers.

In some embodiments, the conjugation of an active agent to a peptide having multiple functionalities such as HR97 is achieved by a maleimide-thiol click reaction. An exemplary conjugation of HR97 to the quaternary-ammonium-linked brimonidine via a thiol-maleimide reaction is shown in Formula I. Another exemplary conjugation of HR97 to the quaternary-ammonium-linked sunitinib via a thiol-maleimide reaction is shown in II.

In some embodiments, conjugation of MC-Val-Cit-PAB-brimonidine or MC-Val-Cit-PAB-sunitinib to HR97 with cysteine at the C-terminus as the functional group for linker conjugation (FSGKRRKRKPRC, Mw=1519, >97% purity) is achieved by a maleimide-thiol click reaction; for example, the MC-Val-Cit-PAB-brimonidine or MC-Val-Cit-PAB-sunitinib is first dissolved in 1 mL of PBS at 5 mg/mL, and HR97 peptide powder (0.5 eq) is added directly to the solution. In some embodiments, the solution mixtures are adjusted to pH 7.4 and allowed to react for 2 h at room temperature. In some embodiments, the solution mixtures are then added to 1 mL of acetonitrile and purified with the same prep-HPLC conditions. In some embodiments, the collected fraction solutions are first transferred to the 20 mL scintillation vials and the Biotage V-10 solvent evaporator with volatile mode are used to remove the acetonitrile. In some embodiments, the solutions are lyophilized and stored at −20 C. Compounds can be confirmed by NMR spectra of Mc-VC-PAB-brimonidine, brimonidine, and Mc-VC-PAB-Cl, respectively. In some embodiments, the compounds are dissolved in deuterated DMSO and run with Bruker spectrometer (500 MHz). In one embodiment, the prep-HPLC retention time (RT) of brimonidine, Mc-VC-PAB-brimonidine, and Mc-VC-PAB-Cl is 5.1, 9.8, and 11.4 min, respectively. In some embodiments, Mc-VC-PAB-sunitinib, sunitinib, and Mc-VC-PAB-Cl are confirmed by NMR spectra. In some embodiments, the compounds are dissolved in deuterated DMSO and ran with Bruker spectrometer (500 MHz). In one embodiment, the prep-HPLC retention time (RT) of sunitinib, Mc-VC-PAB-sunitinib, and Mc-VC-PAB-Cl was 5.4, 8.3, and 11.4 min, respectively.

### V. Methods of Use

Methods of using peptide having multiple functionalities are described. The formulation can be used to treat or prevent one or more disease or disorders, or to treat or prevent one or more symptoms of one or more diseases or disorders in a subject in need thereof. In some embodiments, the formulations deliver an effective amount of an agent to achieve a desired physiological goal or change in the subject. Exemplary physiological changes include variations of the amounts of one or more biomarker in the subject, for example, in the eye of the subject. The change(s) and/or desired outcome of treatment can be monitored to assess the efficacy of treatment, and to determine the amount and extent of treatment required at any given point.

Typically, the peptide having multiple functionalities is conjugated to one or more active agents. The compositions are suitable for treating one or more diseases, conditions, and injuries in the eye. In some embodiments, the compositions are suitable for treating one or more diseases, conditions, and injuries in the skin such as melanoma. The compositions and methods are also suitable for prophylactic use. In preferred embodiments, the compositions of peptide-active agent conjugates are administered in an amount effective to treat one or more diseases, conditions, and injuries in the eye in the subject in need thereof without any associated toxicity.

Delivery of active agents via conjugation to the peptide having multiple functionalities to the eye can treat and prevent diseases and disorders of the eye over a sustained period following a single administration. Typically, administration of the peptide-active agent conjugate provides enhanced therapeutic benefit to the subject as compared with the same amount of the active agent administered to the subject without conjugating to the peptide having multiple functionalities.

The prolonged binding of the peptide to cellular targets such as melanin and steady dissociation of peptide-active agent conjugate into molecules of active agents within the eye provides sustained release of active agents within the confined compartment of the eye, providing long-lasting therapeutic efficacy following a single administration.

In some embodiments, administration of a single dose of peptide-active agent conjugates into the eye provides sustained release of the active agents within the eye for a period of days, weeks or months following administration. Therefore, in some embodiments, administration of a single dose of peptide-active agent conjugates into the eye treats or prevents one or more diseases, conditions, or injuries of the eye for a period of one, two, three, four, five, six, seven, eight, nine or ten, or twenty days or weeks or months following administration. In other embodiments, administration of a single dose of peptide-active agent conjugates into the eye reduces or prevents one or more symptoms of eye disease, or a condition or injury of the eye for a period of one, two, three, four, five, six, seven, eight, nine or ten, or twenty days or weeks or months following administration. In preferred cases, the compositions of peptide-active agent conjugate are administered once a week or less frequently, once every other week or less frequently, once every month or less frequently, once every other month or less frequently.

Typically, the amount of the active agent administered as peptide-active agent conjugates effective to treat or prevent one or more diseases, conditions, or injuries of the eye is less than the amount of the same active agent effective to treat or prevent one or more diseases, conditions, or injuries of the eye as free agents, i.e., without conjugating to the peptide.

In some embodiments, administering compositions of peptide-active agent conjugate increases the concentration of one or more active agents at or near the site of application compared to active agents delivered without the peptides. When the compositions of peptide-active agent conjugate are applied to the eye, the mucosal sites with increased concentration of active agents include one or more of cornea, aqueous humor, sclera, conjunctiva, iris, lens, retina, and retinal pigment epithelium. In some embodiments, the peptide-active agent conjugate compositions are administered topically to the ocular surface of the eye. In some embodiments, the peptide-active agent conjugate compositions are administered by injection into the posterior compartment of the eye.

In some embodiments, formulations of peptide-active agent conjugates in a pharmaceutically acceptable excipient are administered locally into the eye of a subject having an eye disease or disorder, or at risk of having an eye disease or disorder. In a preferred embodiment, the peptide-active agent conjugate formulations are delivered directly into one or more compartments of the eye by injection. In some embodiments, the formulations are injected as a suspension in solution, in a volume of between about 0.1 μl and about 1,000 μl, preferably between about 1 μl and 50 μl. In a particular embodiment, the methods deliver formulations of HR97-brimonidine and HR97-sunitinib by intravitreal, sub-conjunctival, or supra-choroidal injection into the eye for treatment and prevention of an eye disease, such as glaucoma.

In some embodiments, the subject to be treated is a human. In some embodiments, the subject to be treated is a child, or an infant. All the methods can include the step of identifying and selecting a subject in need of treatment, or a subject who would benefit from administration with the described compositions.

### A. Methods of Administering

In some embodiments, peptide-active agent conjugates are administered as gel-forming compositions, preferably as hypotonic gel-forming compositions for further prolonged residence at the site of application.

When the hypotonic gel-forming compositions of the peptide-active agent conjugates are applied to the eye, the mucosal sites with increased concentration of active agents include one or more of cornea, aqueous humor, sclera, conjunctiva, iris, lens, retina, and retinal pigment epithelium.

In some embodiments, the hypotonic gel-forming compositions deliver active agents (e.g., acriflavine and sunitinib malate) to retina and/or choroid in an amount effective to reduce retinal and/or choroidal neovascularization by 10%, 20%, 30%, 40%, 50%, or more than 50% compared to active agents delivered without gel-forming vehicles, for example, in saline solution.

In other embodiments, the hypotonic gel-forming compositions deliver active neuroprotective agents (e.g., sunitinib malate) to the retina in an amount effective to increase the survival of retinal ganglion cells following optic nerve injury, and/or to increase the expression of γ-synuclein and/or βIII tubulin in retinal ganglion cells following optic nerve injury by 2-fold, 3-fold, 4-fold, 5-fold, or more than 5-fold compared to active agents delivered without gel-forming vehicles, for example, in saline solution.

In further examples, the hypotonic gel-forming compositions deliver active agents (e.g., brinzolamide, brimonidine) to the eye in an amount effective to lower intraocular pressure (TOP) by 10%, 20%, 30%, 40%, 50%, or more than 50% compared to those delivered without gel-forming vehicles, for example, in saline solution within less than 2 hours, 4 hours, 6 hours, 8 hours, 10 hours, 12 hours, or 24 hours.

In other examples, the hypotonic gel-forming compositions deliver active agents (e.g., Cyclosporine A) to the eye in an amount effective to increase tear production by 10%, 20%, 30%, 40%, 50%, or more than 50% compared to those delivered without gel-forming vehicles, for example, in saline solution within less than 2 hours, 4 hours, 6 hours, 8 hours, 10 hours, 12 hours, or 24 hours.

In some embodiments, formulations of hypotonic gel-forming compositions including therapeutic, prophylactic, nutraceutical or diagnostic agents are administered as an eye drop into the eye of the subject.

In some embodiments, the administration to the eye is repeated once or more as part of a treatment regimen, for example, to provide a sufficient concentration of agent(s) at or in the eye. Therefore, in some embodiments, the administration to the eye is repeated at a time selected from hourly, daily, every other day, every three days, every four days, every five days, every six days, weekly, every two weeks, or less often.

### B. Eye Disorders and Diseases to be Treated

The compositions and methods are suitable for treating or preventing symptoms of one or more diseases, disorders, and conditions associated with the eye. The compositions and methods are particularly suited for treating and alleviating one or more symptoms associated with glaucoma. In some embodiments, the disclosed compositions or formulations thereof are used to alleviate pain, facilitate healing, and/or reduce or inhibit scarring.

Examples of eye disorders that may be treated according to the disclosed compositions and methods include amoebic keratitis, fungal keratitis, bacterial keratitis, viral keratitis, onchocercal keratitis, bacterial keratoconjunctivitis, viral keratoconjunctivitis, corneal dystrophic diseases, Fuchs' endothelial dystrophy, meibomian gland dysfunction, anterior and posterior blepharitis, conjunctival hyperemia, conjunctival necrosis, cicatricial scaring and fibrosis, punctate epithelial keratopathy, filamentary keratitis, corneal erosions, thinning, ulcerations and perforations, Sjogren's syndrome, Stevens-Johnson syndrome, autoimmune dry eye diseases, environmental dry eye diseases, corneal neovascularization diseases, post-corneal transplant rejection prophylaxis and treatment, autoimmune uveitis, infectious uveitis, anterior uveitis, posterior uveitis (including toxoplasmosis), pan-uveitis, an inflammatory disease of the vitreous or retina, endophthalmitis prophylaxis and treatment, macular edema, macular degeneration, age-related macular degeneration, proliferative and non-proliferative diabetic retinopathy, hypertensive retinopathy, an autoimmune disease of the retina, primary and metastatic intraocular melanoma, other intraocular metastatic tumors, open angle glaucoma, closed angle glaucoma, pigmentary glaucoma and combinations thereof. Other disorders including injury, burn, or abrasion of the cornea, cataracts and age-related degeneration of the eye or vision associated therewith.

In some cases, the eye disorder to be treated are retinal diseases. Retinal diseases can affect any part of the retina, a thin layer of tissue on the inside back wall of the eye. Exemplary retinal diseases include retinal tear, retinal detachment, diabetic retinopathy, epiretinal membrane, macular hole, macular degeneration, and retinitis pigmentosa. In preferred case, the eye disorder is age-related macular degeneration (AMD). Age-related macular degeneration (AMD) is a neurodegenerative, neuroinflammatory disease of the macula, which is responsible for central vision loss. The pathogenesis of age-related macular degeneration involves chronic neuroinflammation in the choroid (a blood vessel layer under the retina), the retinal pigment epithelium (RPE), a cell layer under the neurosensory retina, Bruch's membrane and the neurosensory retina, itself.

The formulations may be administered to animals, especially mammalian animals for treating or alleviating pain, disease, disorder, infection, or injury of the eye.

In some embodiments, the methods treat or prevent a disease or disorder including glaucoma, dry eye syndrome (DES), macular degeneration, diabetic retinopathy, scleroderma, and cancer.

In preferred embodiments, the formulations of the peptide-active agent conjugates, preferably as hypotonic gel-forming formulations, are administered to the surface of the eye of a subject for the treatment of macular degeneration, especially wet macular degeneration or Stargardt disease. Stargardt disease is a form of macular degeneration found in young people, caused by a recessive gene.

Age-related macular degeneration (AMD) is a common condition that affects the middle part of the vision. It usually first affects people in their 50s and 60s, or older. There are two basic types of macular degeneration: “dry” and “wet.” Approximately 85% to 90% of the cases of macular degeneration are the “dry” (atrophic) type, while 10-15% are the “wet” (exudative) type.

Dry macular degeneration is a common eye disorder among people over 50. It causes blurred or reduced central vision, due to thinning of the macula. The macula is the part of the retina responsible for clear vision in the direct line of sight.

In other embodiments, the formulations of the peptide-active agent conjugates, preferably as hypotonic gel-forming formulations, are administered to the surface of the eye of a subject for the treatment of wet macular degeneration in the eye of the subject. In the “wet” type of macular degeneration, abnormal blood vessels (known as choroidal neovascularization or CNV) grow under the retina and macula. These new blood vessels may then bleed and leak fluid, causing the macula to bulge or lift up from its normally flat position, thus distorting or destroying central vision. Under these circumstances, vision loss may be rapid and severe. VEGF is an acronym for vascular endothelial growth factor. Currently, the most common and effective clinical treatment for wet Age-related Macular Degeneration is anti-vascular endothelial growth factor (anti-VEGF therapy) using an inhibitor of VEGF (anti-VEGF). VEGF is a molecule which supports the growth of new blood vessels. In the case of wet AMD, VEGF promotes the growth of new, weak blood vessels in the choroid layer behind the retina, and those vessels leak blood, lipids, and serum into the retinal layers. The leakage (hemorrhaging) causes scarring in the retina and kills macular cells, including photoreceptor rods and cones. Therefore, in some embodiments, the formulations including one or more anti-VEGF agents are administered to the surface of the eye of a subject for the treatment of wet macular degeneration in the eye of the subject.

In other embodiments, the formulations of the peptide-active agent conjugates, preferably as hypotonic gel-forming formulations, are administered to the surface of the eye of a subject for the treatment of Stargardt disease. Stargardt disease is a form of macular degeneration found in young people, caused by a recessive gene.

In some embodiments, the formulations of the peptide-active agent conjugates, preferably as hypotonic gel-forming formulations, are administered to the surface of the eye of a subject for the treatment of dry eye syndrome (DES) in the eye of the subject. Dry eye syndrome (DES), also known as keratoconjunctivitis sicca (KCS), or dry eye disease, is a common condition that occurs when the eyes do not make enough tears, or the tears evaporate too quickly. Other associated symptoms include irritation, redness, discharge, and easily fatigued eyes. Blurred vision may also occur. Symptoms range from mild and occasional to severe and continuous. Scarring of the cornea may occur in untreated cases. The cornea includes the clear outer dome of the eye that allows light to enter and become focused through the lens onto the retina. The cornea is an avascular tissue and receives most of its nutrients from the tears, the air, and fluid inside the eye.

In some embodiments, the formulations of the peptide-active agent conjugates, preferably as hypotonic gel-forming formulations, are administered to the surface of the eye of a subject for the treatment of dry eye syndrome (DES) in the eye of the subject.

### C. Dosage and Effective Amounts

Dosage and dosing regimens are dependent on the severity and location of the disorder or injury and/or methods of administration, and can be determined by those skilled in the art. A therapeutically effective amount of the peptide-active agent conjugate composition used in the treatment of eye disorders and/or diseases is typically sufficient to reduce or alleviate one or more symptoms of the eye disorders and/or diseases in the subject. Exemplary symptoms include eye pain or eye discomfort, such as soreness, dryness, burning, stinging or aching sensations, reduced vision loss or visual acuity, reduced ability to read or interpret colors, reduced movement of the eye, or one or more physiological parameters, such as expression of γ-synuclein and/or βIII tubulin in retinal ganglion cells, increased intra-ocular pressure, vascular leakage from retinal blood vessels, and retinal and/or choroidal neovascularization.

Preferably, the active agents do not target or otherwise modulate the activity or quantity of healthy cells not within or associated with the diseased/damaged tissue, or do so at a reduced level compared to cells associated with the diseased/damaged eye tissue. In this way, by-products and other side effects associated with the compositions are reduced.

Therefore, administration of the peptide-active agent conjugate compositions leads to an improvement, or enhancement, function in an individual with an ocular disease, injury, or disorder. The actual effective amounts of the peptide-active agent conjugate compositions can vary according to factors including the specific agent administered, the particular composition formulated, and the age, weight, condition of the subject being treated, as well as the disease or disorder. Dosage can vary, and can be administered in one or more dose administrations weekly, monthly, bimonthly, once every six month or less frequently. Guidance can be found in the literature for appropriate dosages for given classes of pharmaceutical products. Optimal dosing schedules can be calculated from measurements of drug accumulation in the body and/or eye of the subject or patient. Persons of ordinary skill in the art can easily determine optimum dosages, dosing methodologies and repetition rates. Optimum dosages can vary depending on the relative potency of individual pharmaceutical compositions and can generally be estimated based on EC₅₀ levels found to be effective in in vitro and in vivo animal models.

Dosage forms of the pharmaceutical composition including peptide-active agent conjugates are also provided. “Dosage form” refers to the physical form of a dose of a therapeutic compound, such as a capsule or vial, intended to be administered to a patient. The term “dosage unit” refers to the amount of the active agents to be administered to a patient in a single dose.

In some embodiments, the dosage unit suitable for use are between 5 μg/dosage unit and about 100 mg/dosage unit, inclusive; between about 10 μg/dosage unit and about 20 mg/dosage unit, inclusive; and between about 100 μg/dosage unit and about 10 mg/dosage unit, inclusive; and between about 200 μg/dosage unit and about 5 mg/dosage unit, inclusive; and between about 500 μg/dosage unit and about 3 mg/dosage unit, inclusive.

It will be understood by those of ordinary skill that a dosing regimen can be any length of time sufficient to treat the disease or disorder in the subject. In some embodiments, the regimen includes one or more cycles of a round of therapy followed by a drug holiday (e.g., no drug). The drug holiday can be 1, 2, 3, 4, 5, 6, or 7 days; or 1, 2, 3, 4 weeks, or 1, 2, 3, 4, 5, or 6 months.

### D. Combination Therapies and Procedures

The peptide-active agent conjugate compositions can be administered alone or in combination with one or more conventional therapies. In some embodiments, the conventional therapy includes administration of the peptide-active agent conjugate compositions in combination with one or more additional active agents. The combination therapies can include administration of the active agents together in the same admixture, or in separate admixtures. Therefore, in some embodiments, the pharmaceutical composition includes peptide-active agent conjugate and one or more additional active agent. The one or more additional active agent can be administered in the same pharmaceutical formulation, or in a separate formulation delivered at the same time, or at a different time as the microcrystals. In some embodiments, the treatment regimen includes multiple administrations of peptide-active agent conjugates to the same eye, or to different eyes. The additional active agent(s) can have the same or different mechanisms of action. In some embodiments, the combination results in an additive effect on the treatment of the eye disease or condition. In some embodiments, the combinations result in a more than additive effect on the treatment of the disease or disorder.

In some embodiments, the compositions and methods are used prior to or in conjunction, simultaneously, subsequent to, or in alternation with treatment with one or more additional therapies or procedures.

The additional therapy or procedure can be simultaneous or sequential with the administration of the peptide-active agent conjugate composition. In some embodiments, the additional therapy is performed between drug cycles or during a drug holiday that is part of the composition's dosage regime. Therefore, in some embodiments, a formulation of the peptide-active agent conjugate is administered in a single dose to a subject 1, 2, 3, 4, 5, 6, 7, 8, 9 or 10 minutes, hours or days before or after administration of an additional active agent to the same subject for the treatment of the same eye disease or condition.

### E. Controls

The therapeutic effect of peptide-active agent conjugate compositions including one or more active agents can be compared to a control. Suitable controls are known in the art and include, for example, an untreated subject or a placebo-treated subject, or treatment with the same therapeutic, prophylactic or diagnostic agent without the targeting peptide. A typical control is a comparison of a condition or symptom of a subject prior to and after administration of the targeted agent. The condition or symptom can be a biochemical, molecular, physiological, or pathological readout. For example, the effect of the peptide-active agent conjugate composition on a particular symptom, pharmacologic, or physiologic indicator can be compared to an untreated subject, or the condition of the subject prior to treatment, or to the same amount of active agent without conjugating to the peptide. In some embodiments, the symptom, pharmacologic, or physiologic indicator is measured in a subject prior to treatment, and again one or more times after treatment is initiated. In some embodiments, the control is a reference level, or average determined based on measuring the symptom, pharmacologic, or physiologic indicator in one or more subjects that do not have the disease or condition to be treated (e.g., healthy subjects). In some embodiments, the effect of the peptide-active agent conjugate treatment is compared to a conventional treatment that is known the art. In some embodiments, an untreated control subject suffers from the same eye disease or condition as the treated subject.

### VI. Kits

The compositions can be packaged in kit. The kit can include a single dose or a plurality of doses of a composition including one or more active agents and instructions for administering the compositions. Specifically, the instructions direct that an effective amount of the composition be administered to an individual with a particular eye disease/disorder as indicated. The composition can be formulated as described above with reference to a particular treatment method and can be packaged in any convenient manner. These may be unit dosage forms such as a pre-loaded syringe and one or more needles with gauge suitable for administration to the eye, for example, 25-, 27- or 30-gauge needles. In some embodiments, dosage formulations are prepared as single or multiple liquid or dry dosage units in an appropriate applicator, for example, an eye drop dispenser.

The present invention will be further understood by reference to the following non-limiting examples.

### EXAMPLES

### Example 1: Design of Multifunctional Peptides that can be Conjugated to Drugs to Impart Sustained Intraocular Delivery

For effective, targeted and/or enhanced delivery of active agents to the eye, carriers such as peptide molecules or drug conjugates are required. One target for increased delivery to the eye, and residence therein, is based on the presence of melanin, which is used as a target. An ideal targeting agent for melanin would possess multiple functionalities, including: (i) high binding to melanin (pigment in the eye); (ii) high cell-penetration (to enter cells and access melanin in the melanosomes); and (ii) low cytotoxicity. However, melanin binding can affect ocular drug biodistribution. Due to the low turnover rate of ocular melanin, drug that can bind to melanin may accumulate in pigmented eye tissues, leading to drug toxicity or drug sequestration (Rimpela, et al., Adv Drug Deliv Rev 126, 23-43, (2018)).

Attempts to custom-design molecules having multiple functions for drug delivery to the eye are hampered by technical challenges, and thus multifunctional peptides are often designed by fusing peptides via a linker, with less efficient rational design, or by testing additional properties on peptides with known functions (Cheng, et al., ACS Appl Mater Interfaces 9, 2093-2103, (2017); Drexelius, et al., Biochem J 478, 63-78, (2021); Felicio, et al., Front Chem 5, 5, (2017)).

The following studies were conducted to address both issues.

### Methods and Materials

### Material Sources

Sunitinib malate was purchased from LC Laboratories. Brimonidine was purchased from TCI America. Eumelanin from Sepia officinalis, 0.22 μm Millex-GV PVDF filter, ferric ammonium citrate, bovine serum albumin (BSA), Tween 20, fetal bovine serum (FBS), Lutrol F127, trifluoroacetic acid (TFA), tert-Butyl methyl ether (MTBE), thionyl chloride, Tetrabutylammonium iodide, N,N-diisopropylethylamine, human cathepsins B, K, L and S, WHATMAN® ANOTOP® 0.02 μm syringe filter and Triton X-100 were purchased from Sigma Aldrich (St. Louis, MO, USA). ARPE-19 (CRL-2302), and DMEM:F12 medium was purchased from the American Type Culture Collection (Manassas, VA, USA). EZ-LINK™ Amine-PEG₂-Biotin, BupH MES buffer saline pack (2-(N-morpholino)ethanesulfonic acid) buffer), EDC (1-ethyl-3-(3-dimethylaninopropyl)carbodiimide hydrochloride), NHS (N-hydroxysuccinimide), Pierce™ Fluorescence Biotin Quantitation Kit, rapid equilibrium dialysis (RED) 8 k device, DMEM with high glucose and pyruvate, Trypsin-EDTA (0.25%) with phenol, RIPA lysis buffer, RBPMS rabbit anti rat polyclonal antibody, Alexa Fluor 555 conjugated goat anti-rabbit IgG (H+L) secondary antibody, penicillin/streptomycin, 4′,6-diamidino-2-phenylindole, dihydrochloride (DAPI), Fluoromount-G, IMAGE-IT™ Fixative Solution (4% formaldehyde, methanol-free) were purchased from Thermo Fisher Scientific (Waltham, MA, USA). Disposable PD-10 desalting columns was purchased from WVR. Dulbecco's Phosphate Buffered Saline (DPBS), 1×phosphate buffered saline (PBS), 10×PBS, high-performance liquid chromatography (HPLC) grade acetonitrile, dimethylformamide (DMF), and water were purchased from Fisher Scientific (Hampton, NH, USA). Mc-Val-Cit-PAB was purchased from Cayman Chemical (Ann Arbor, MI, USA). Endotoxin-Free Ultra-pure Water were purchased from EMD Millipore Sigma (Burlington, MA, USA). A Hamilton 1700 Series gas tight syringes (25 μL, Model 1702 RN, 27 gauge) was purchased from Hamilton Company (Reno, NV, USA). BD 1 mL TB syringe with 28 G needles were purchased from BD (San Jose, CA, USA). Isoflurane was purchased from Baxter (Deerfield, IL, USA). Reverse-action forceps were purchased from World Precision Instruments (Sarasota, FL, USA). Neomycin, polymyxin b, and bacitracin zinc ophthalmic ointment was purchased from Akorn (Lake Forest, IL, USA).

### Melanin Nanoparticle Synthesis and Characterization

Melanin nanoparticles (mNPs) were synthesized from the eumelanin of Sepia officinalis. In brief, 10 mg/mL of eumelanin was suspended in the DPBS using an ultrasonic probe sonicator (Sonics, Vibra Cell VCX-750 with model CV334 probe, Newtown, CT, USA) by pulsing 1 s on/off at 40% amplitude for 30 min in a 4 C water bath. The suspension was then filtered through a 0.22 μm Millex-GV PVDF filter and transferred to PD-10 desalting columns. The resulting mNPs solution was lyophilized for 7 d and stored at −20 C until further use. For mNP biotinylation (b-mNPs), mNPs were suspended in 2 mL MES buffer with 2.4 mg of EDC and 3.6 mg of NHS for 15 min at room temperature (RT) to first activate the carboxylic acid groups. To increase the buffer pH above pH 7.4 for amine reaction, 400 μL of 10×PBS was directly added to the mixture and incubated for 5 min. Various amounts of EZ-LINK™ Amine-PEG₂-Biotin (5, 15, 20, 30 mg) were reacted with activated mNPs for 2 or 6 h at RT. Since all conditions led to similar degree of mNP biotinylation (data not shown), reaction conditions using 5 mg of amine-PEG₂-biotin with 2 h incubation at RT was used moving forward. The reaction mixture was then transferred to PD-10 desalting columns to further collect the b-mNPs. To transfer the b-mNPs to different solvents (water, pH 6.5 PBS, pH 7.4 PBS) for optimization of the peptide microarray, PD10 columns were first equilibrated with buffer, and then the b-mNPs were added. Particle size and -potential were determined by dynamic light scattering and laser Doppler anemometry, respectively, using a Zetasizer Nano ZS90 (Malvern Instruments). Size measurements were performed at 25 C at a scattering angle of 173°. Samples were diluted in 10 mM NaCl solution (pH 7) and measurements performed according to instrument instructions. Pierce™ Fluorescence Biotin Quantitation Kits were used to quantify the biotin content on the b-mNPs. B-mNPs (1 mg/mL) were diluted 1:50, 1:100, 1:200 with 1×PBS and the standard biocytin concentration (10-60 pmol/10 μL) were freshly prepared for measuring the biotin concentration. Transmission electron microscopy (H7600; Hitachi High Technologies America) was conducted to determine the morphology of mNPs and b-mNPs.

### Optimization of Processing Conditions for Peptide Microarray

A total of 119 peptides, including 8 peptides of length 7 aa, and 8 peptides of length 10 aa from the literature (Howell, R. C. et al., Bioconjug Chen 18, 1739-1748 (2007); and Nosanchuk, J. D., et al., Mol Cell Biol 19, 745-750 (1999)), and 103 random 15 aa peptides generated with a frequency of 5% for each of the 20 amino acids, were printed in duplicate on peptide microarrays by PEPPERPRINT. The peptide microarrays contained hemagglutinin (HA) peptides (YPYDVPDYAG; 9 spots) as internal quality controls. All screening conditions of the peptide microarray results were performed in duplicate arrays. Two peptide microarray copies were first pre-stained with streptavidin DyLight680 (0.2 μg/ml) and the control antibody (mouse monoclonal anti-HA (12CA5) DyLight800; 0.5 μg/ml) in incubation buffer (pH 6.5 PBS with 0.005% Tween 20 and 10% Rockland blocking buffer MB-070) for 45 min at room temperature to examine background interactions and internal quality control. No background interaction of streptavidin DyLight680 or the control antibody with the 119 different peptides were observed. To screen the optimal melanin binding condition, six different washing buffers were prepared: PBS at pH 6.5 with or without 0.005% Tween 20, PBS at pH 7.4 with or without 0.005% Tween 20, Ultra-pure water with or without 0.005% Tween 20. The Rockland blocking buffer MB-070 were used to incubate all peptide microarrays for 30 minutes before the melanin binding assay. Six different incubation buffers were formulated with 10% of blocking buffer in the six different washing buffers mentioned earlier. b-mNPs (10, 100, or 500 μg/ml) in six different incubation buffers were incubated with the peptide microarray for 16 hours at 4 C or room temperature. All microarrays were subsequently washed with the same type of washing buffers and incubated with 0.2 μg/mL of streptavidin DyLight680 for 45 min in the same type of incubation buffer at room temperature for detecting the b-mNPs. The peptide microarrays were then washed for 3×10 sec with same type of washing buffers and proceed to quantification of spot intensity. Quantification of spot intensities and peptide annotation were based on the 16-bit gray scale Tag Image File Format files that exhibit a higher dynamic range than the 24-bit colorized Tag Image File Format files. Microarray image analysis was done with PEPSLIDE® Analyzer (version 1.4). The software algorithm decomposed fluorescence intensities of each spot into raw, foreground and background signal, and calculated averaged median foreground intensities and spot-to-spot deviations of spot duplicates. Based on averaged median foreground intensities, intensity maps were generated and interactions in the peptide maps highlighted by an intensity color code with red for high and white for low spot intensities. The PEPPERPRINT protocol tolerated a maximum spot-to-spot deviation of 40%, otherwise the corresponding intensity value was zeroed. Based on the distribution of the melanin binding spot intensity, we labeled the top 20% of peptides ranked by intensities as melanin binding (23 peptides), which included the 6 literature reported peptides with non-zero fluorescent signal. The remaining peptides were labeled as non-melanin binding (96 peptides).

Random Forest Classification Model Training Using Data from the Pilot 119 Peptide Microarray

Physiochemical properties and numerical representations of peptides were computed using R, version 4.1.0, with packages Peptides, version 2.4.1 (Osorio, D. & Rondón-Villarrea, P. R Journal 7 (2015)) and protr, version 1.6-2 (Xiao, N., et al., Bioinformatics 31, 1857-1859 (2015)). The resulting 1,094 features include composition, transition, distribution, autocorrelation, conjoint triad, quasi-sequence-order descriptors, as well as pseudo-amino acid and amphiphilic pseudo-amino acid composition descriptors. The maximum value of lag was set to 6, so the minimum length of a peptide to be analyzed without generating a missing value is 7. A random forest classification model with 100,000 trees and balanced sampling was trained on the melanin binding dataset containing 1,094 peptide features. The model was built using the R package randomForest, version 4.6-14 (Liaw, A. & Wiener, R news 2, 18-22 (2002)). For each tree in the random forest, a bootstrap sample of approximately 63.2% of the melanin binding peptides and the same amount of non-melanin binding peptides was generated to construct the tree. The remaining peptides were considered out-of-bag to the tree and were used to evaluate the performance of the random forest by calculating the aggregated out-of-bag predictions across all trees. The out-of-bag class errors were calculated and a classification threshold of 0.5 proportion of votes was used. As part of the same analysis, permutation feature importance was obtained with the function importance in the random Forest package. For each tree in the random forest, out-of-bag instances were permutated for each feature, and the decrease in accuracy were recorded. The mean decrease in accuracy for each feature was calculated over all 100,000 trees and normalized by dividing the mean by the standard deviation.

### Expansion of the Peptide Microarray

Melanin binding candidate peptides were generated randomly with a frequency of 5% for each of the 20 amino acids. Peptides classified as melanin binding by the trained random forest model were selected, resulting in 5,483 peptides of lengths between 7 and 12 amino acids. Along with the 16 known melanin binding peptides from the literature, a total of 5,499 peptides were printed in duplicate along with HA controls (YPYDVPDYAG; 68 spots) on peptide microarrays by PEPPERPRINT. Peptide sequences were printed in duplicate of a custom peptide microarray. Pre-staining of a peptide microarray copy was done with streptavidin DyLight680 (0.2 μg/ml) and the control antibody (mouse monoclonal anti-HA (12CA5) DyLight800; 0.5 μg/ml) in incubation buffer to characterize non-specific binding. Subsequent incubation of another peptide microarray copy with the b-mNPs at a concentration of 500 μg/ml in incubation buffer (PBS at pH 6.5 with 0.005% Tween 20 with 10% Rockland blocking buffer MB-070) was followed by staining with streptavidin DyLight680 (0.5 μg/mL) and the control antibody (0.5 μg/mL). The control staining of the HA epitopes was done simultaneously as internal quality control to confirm the assay quality and the peptide microarray integrity. Quantification of spot intensities were described earlier in the previous section.

### Super Learner Model Training and Refinement

To generate the machine learning input dataset, peptide features were analyzed as described above. Melanin binding was normalized by setting the maximum value to 100, the minimum value to 0, and other values adjusted accordingly. To reduce the number of features and improve the model performance, a recursive feature elimination procedure was applied to the dataset containing 5,499 peptides and 1,094 features. The dataset was first imputed by replacing missing values with feature medians and most frequent category for numeric and categorical features, respectively. Features with missing values for all instances were removed. Next, for each iteration, permutation-based feature importance was computed from a random forest regression model built using the R package ranger, version 0.12.1 (Wright, M. N. & Ziegler, A. ranger: a fast implementation of random forests for high dimensional data in C++ and R. J Stat Softw 77, 1-17 (2017)) with 25,000 trees. Features with importance less than or equal to zero were removed during each iteration.

A super learner (SL) model was built using the R interface of H2O.ai, version 3.30.0.2 (H2O.ai. h2o: R interface for H2O. (2020)). The hyperparameters of the base models were explored using H2O AutoML (LeDell, et al., H2o automl: Scalable automatic machine learning. In Proceedings of the AutoML Workshop at ICML 2020 Jul. 18 (Vol. 2020)). More specifically, the function h2o.automl was employed with the setting of the maximum number of base models being 1,000. The base model types include deep learning, gradient boosting machines, XGBoost, distributed random forest, extremely randomized trees, and generalized linear models. For non-tree-based models, features in the training set were scaled to have zero means and unit variances. Different versions within the same super learning analysis is used because each single model can be built independently. The data was scaled specifically for non-tree-based models.

DBase model hyperparameter combinations were selected randomly from defined grids for each base model type. The search of hyperparameters for each model type stopped when a 3-round moving average of mean residual deviance (for regression) or mean per class error (for classification) calculated with 10-fold cross-validation is less than

$$1 / \sqrt{number of instances}$$

(LeDell, et al., H2o automl: Scalable automatic machine learning. In Proceedings of the AutoML Workshop at ICML 2020 Jul. 18 (Vol. 2020)). Unstable neural network models with potentially large activation values were removed. In total, 891 base models were generated for the melanin binding regression dataset, including 12 base models with default parameters, 745 grid searched deep learning models, 114 grid searched GBM models, and 20 grid searched XGBoost models.

A non-negative least squares algorithm (meta-learner) was used to calculate the coefficients (weighted contributions) of the base models according to their holdout predictions generated from 10-fold cross-validation. The meta-learner was evaluated with another 10-fold cross-validation trained on the base model holdout prediction dataset. Coefficient distributions were collected from the 10 cross-validation meta learner models, resulting in a n×m matrix, where n is the number of base models, and m=10 is the number of cross-validation folds. To reduce the number of base models in the final ensemble and to improve the model performance, base models with the number of zero coefficients larger than 5 were removed. The filtering procedure was repeated until the mean residual deviance (for regression) or mean per class error (for classification) of the SL ensemble does not decrease, or there is no base model with the number of zero coefficients larger than 5. The numbers of base models in the final ensembles were reduced to 24. Ensembles with different compositions of base models were also constructed for comparison. The best-of-family ensemble model was built with the best base models of each model type. Homogeneous ensembles were constructed with base models of the same model type (deep learning, GBM, XGBoost).

Model evaluation was performed with 10-fold cross-validation. The ensembles and their base models were evaluated on holdout prediction datasets and original input datasets, respectively. Regression models were evaluated using multiple metrics, including coefficient of determination (R²), mean absolute error (MAE) that is insensitive to outlines, and normalized MAE, which was calculated by dividing MAE by the maximum observed value.

### Super Learner Model Training to Include Cell-Penetration Predictions

Cell-penetrating and non-cell penetrating peptides of varying lengths (10-61 amino acids) were collected from the SkipCPP-Pred website (Wei, L., Tang, J. & Zou, Q. BMC Genomics 18, 742, (2017)), for which the redundant cell-penetrating peptides from the CPPsite2.0 database (Agrawal, P. et al. CPPsite 2.0: a repository of experimentally validated cell-penetrating peptides. Nucleic Acids Res 44, D1098-1103 (2016)) have been removed, and non-cell penetrating peptides were generated randomly. Peptide features were computed with the same methods mentioned previously for the melanin binding peptides. Preprocessing and recursive feature elimination procedures were then applied to the dataset as described above. The SL classification model was computed with the maximum number of base models set to 1,000. All 1,000 base models were trained with no unstable neural network models having potentially large activation values generated. The trained models include 12 default models, 957 grid searched deep learning models, 12 grid searched GBM models, and 19 grid searched XGBoost models. The number of based models was further reduced to 118 with the iterative filtering procedure described above. Classification models were evaluated with balanced accuracy and log-loss. Prediction thresholds for classification SL models were selected based on the maximum F_1 score, which is the harmonic mean of precision and recall.

### Super Learner Model Training to Include Toxicity Predictions

Toxic and non-toxic peptides of varying lengths (4-35 aa) were collected from the ToxinPred website (Gupta, S. et al. In silico approach for predicting toxicity of peptides and proteins. PLoS One 8, e73957, (2013)). Peptides with length less than 7 were excluded, resulting in 1,777 toxic and 3,522 non-toxic peptides. Peptide features were calculated with the same methods used for melanin binding peptides. The dataset was further preprocessed, and the features were filtered as described above. The SL classification model was computed with the maximum number of base models set to 1,000. There were no unstable models generated, and 1,000 base models were constructed, with 12 default models, 856 grid searched deep learning models, 25 grid searched GBM models, and 107 grid searched XGBoost models. The final SL ensembled contains 28 base models. Base models and the final SL ensemble were evaluated as described in the above cell-penetration SL training section.

### Results

With the right balance of melanin-binding affinity and capacity, melanin may act as a sustained-release depot in the eye that results in prolonged therapeutic action (Kim, et al., Drug Deliv Transi Res, (2021); Wei, et al., BMC Genomics 18, 742, (2017)). In order to impart beneficial melanin-binding properties to drugs, a tunable approach for engineering peptides with high melanin binding that could be conjugated to small molecule drugs through a reducible linker. Thus, the peptide would provide enhanced retention time, while the linker would ensure that drug could be released and exert its therapeutic action in a sustained manner. However, although there are available databases describing how peptide sequence affects cell-penetration (Wei, L., et al., BMC Genomics 18, 742, (2017); and Agrawal, P. et al., Nucleic Acids Res 44, D1098-1103, (2016)) and separately cytotoxicity (Gupta, S. et al. PLoS One 8, e73957, (2013)), there was no prior information for how peptide sequences affect melanin binding. Thus, a melanin binding peptide microarray was developed to experimentally determine the effect of peptide sequence on melanin binding. An SL regression model was then used to identify peptide sequences that display all three desired properties enumerated above. Importantly, with the Shapley additive explanation (SHAP) analysis 26 of peptide features, the machine learning model interpretation provided additional insights and reasoning for engineering the multifunctional peptides.

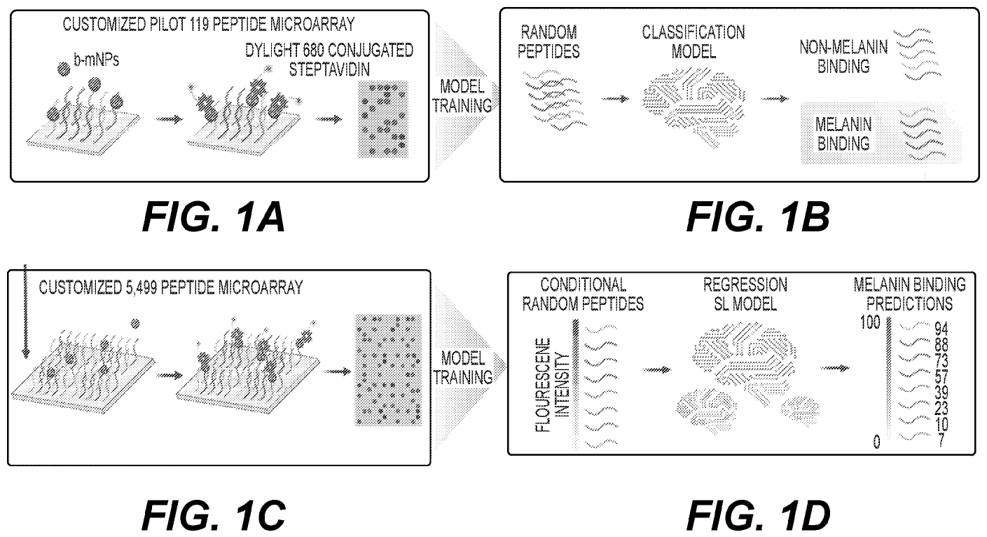

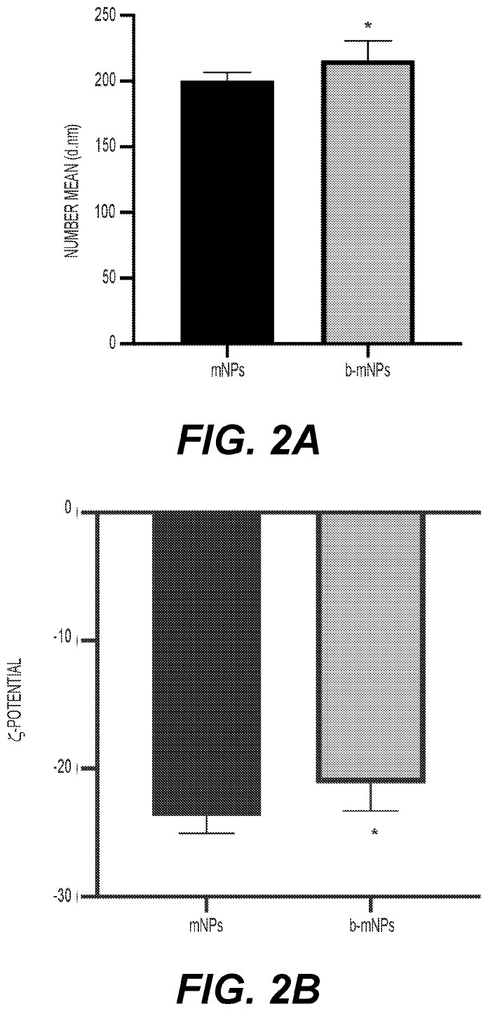

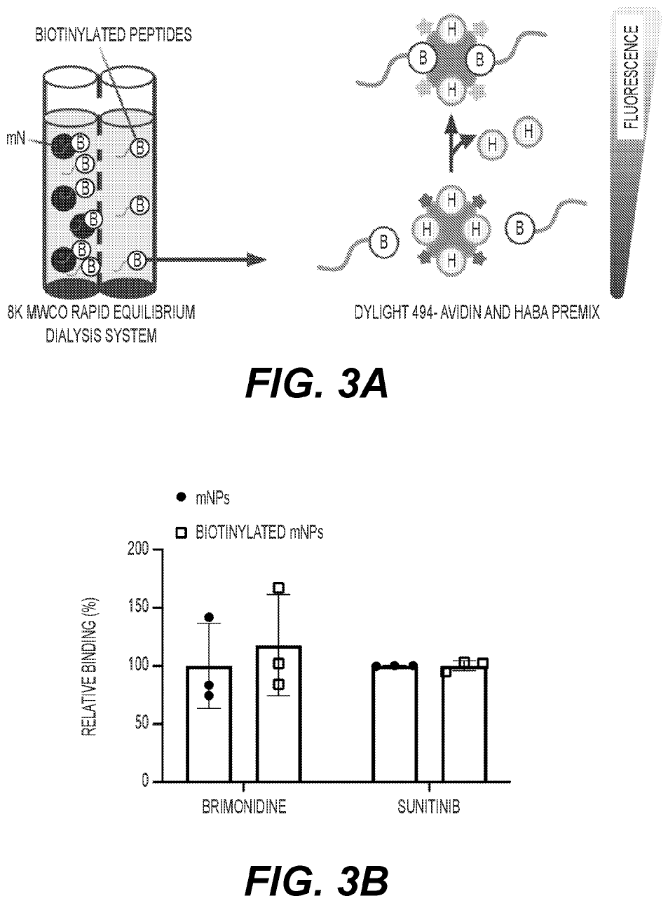

Here, a platform has been developed including experimental screening and computational modeling to engineer multifunctional peptides with a high degree of melanin binding, high cell-penetration, and low cytotoxicity. To develop a machine learning model to predict melanin binding, first a dataset was established that correlated peptide sequence with binding to melanin. A schematic overview of the process is shown in FIGS. 1A-1D. To generate sufficient data for use in training a machine learning model, PEPPERPRINT® was used to develop a high-throughput flow-based microarray system to screen peptides for melanin binding. In the microarray system, peptides are printed onto the substrate surface, and potential binding moieties are then suspended in fluid that is flowed through the device. Thus, an approach was first devised for processing melanin for use in the flow system that also allowed for detection of the amount of melanin that became bound. Commercially available eumelanin powder typically contains particulates of irregular size, leading to a range of surface area available for binding for a given mass concentration of melanin. Further, larger melanin particulates may sediment in a flow system, potentially leading to false positives. Thus, melanin was processed into nanoparticles (mNPs) with an average size of 200.7±5.99 nm and ζ-potential of −23.7±1.39 mV (FIGS. 2A-2B). Since there is no specific antibody designed for melanin, the mNPs (b-mNPs) were biotinylated and the streptavidin DyLight680 reporter were used for quantification of melanin bound to peptides in the peptide microarray system. Biotin was selected as the melanin reporter because of the relatively small molecular size (Mw=244.31 g/mol) compared to other fluorescence reporters, and high binding specificity (K_{d}=10⁻¹⁵ M) for high sensitivity. The b-mNPs showed slightly higher average size of 216.0±14.85 nm and ζ-potential of −21.2±2.15 mV (FIGS. 2A-2B). To confirm that the surface biotinylation did not significantly alter binding, it was confirmed that brimonidine and sunitinib showed similar binding to both mNPs and b-mNPs (FIGS. 3A-3B). Thus, the b-mNPs were synthesized and used in the high-throughput flow-based melanin microarray system.

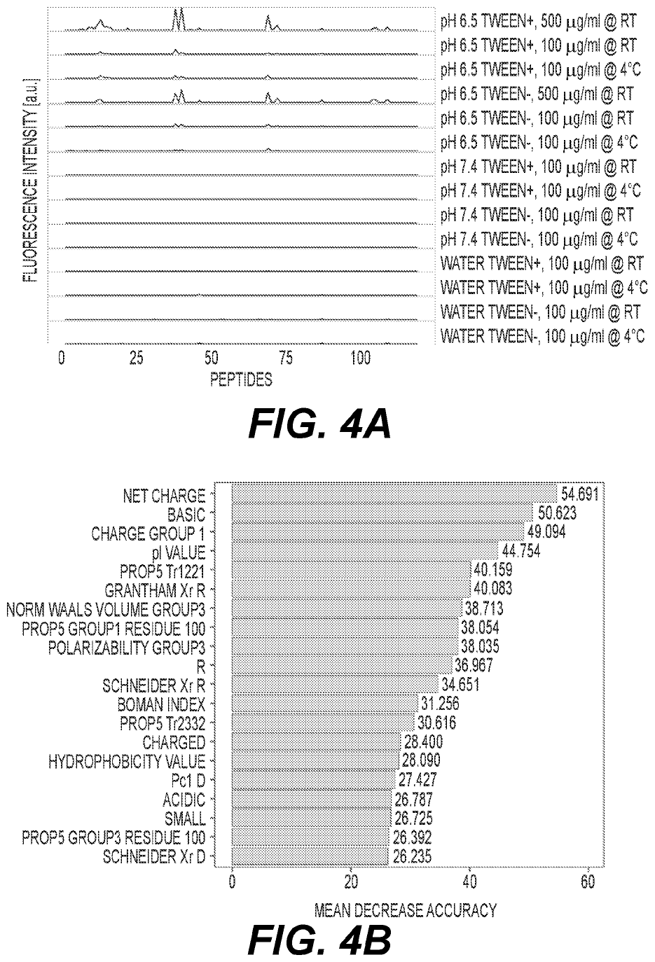

For a first smaller peptide screen, 16 peptides were used that were identified using phage display libraries for murine infection and human melanoma as having melanin binding properties. 103 random peptides generated with a frequency of 5% for each of the 20 amino acids were then added, resulting in a total of 119 peptides that were printed in duplicate on a peptide microarray by PEPPERPRINT®. Various processing conditions were then screened to optimize the fluorescent signal generated by the streptavidin DyLight680 reporter and to minimize non-specific binding (FIG. 4A). The pilot tests suggested that 500 μg/mL of biotinylated mNPs in pH 6.5 PBS buffer at room temperature was optimal (FIG. 4A). With these flow conditions, 10 of the 16 peptides reported in the literature had detectable fluorescence intensities due to binding by b-mNPs (not shown). We labeled the top 20% of peptides ranked by intensities as melanin binding (23 peptides), which included the 6 literature reported peptides with non-zero fluorescent signal. The remaining peptides were labeled as non-melanin binding (96 peptides). A melanin binding classification random forest model was then constructed. Random forest is an ensemble tree-based statistical machine learning model and is robust to feature noise and insensitive to feature scales (Breiman, L. Random forests. Mach Learn 45, 5-32 (2001)). To generate input dataset for model training, numerical representations of peptide sequences including physiochemical compositions of peptides were calculated as input features. The class error of melanin binding was 34.8% and that of non-melanin binding was 3.1%. To understand peptide features that were important in classifying melanin binding peptides, permutation-based feature importance analysis was performed. The results showed that net charge, basic amino acids, and isoelectric point (pI) may contribute to distinguishing melanin binding and non-melanin binding peptides (FIG. 4B).

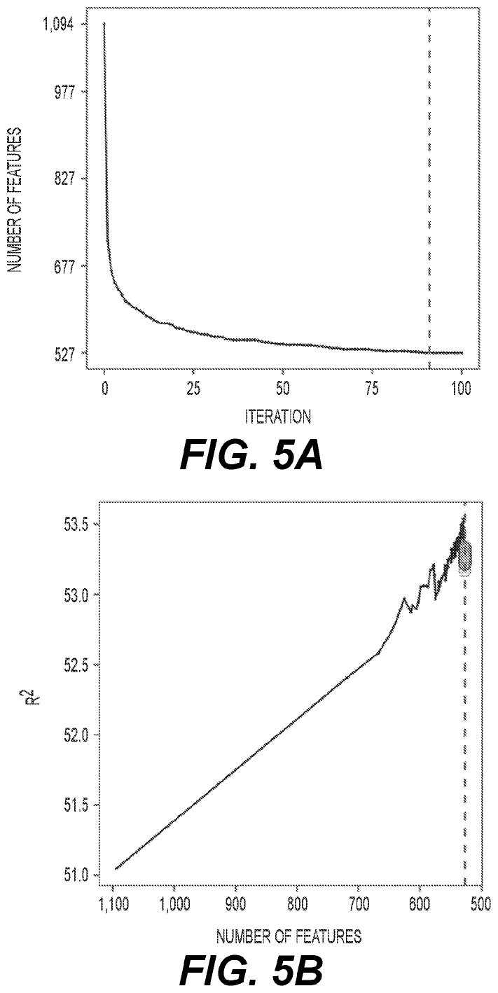

A regression model was then constructed by generating 5,483 random peptides with a 5% frequency for each amino acid. The peptides were of length varying from 7 to 12 aa and were classified as melanin binding or non-melanin binding by the random forest model (FIG. 1B). Along with 16 peptides obtained from the literature, a total of 5,499 peptides were printed in duplicate on the second peptide microarray (FIG. 1C). The fluorescent signals indicative of binding by the b-mNPs were used as the response variable in the regression model. The PEPperPRINT protocol tolerated a maximum spot-to-spot deviation of 40%, otherwise the corresponding intensity value was zeroed. To reduce the number of features in the input dataset and improve the model performance, recursive feature elimination was first performed using random forest (Guyon, et al., Gene selection for cancer classification using support vector machines. Mach Learn, 46(1), 389-422, (2002)). The number of features were reduced from 1,094 to 527 after 200 iterations (FIG. 5A), and the model performance in terms of the coefficient of determination (R²) was improved from 0.511 to 0.533 (FIG. 5B).

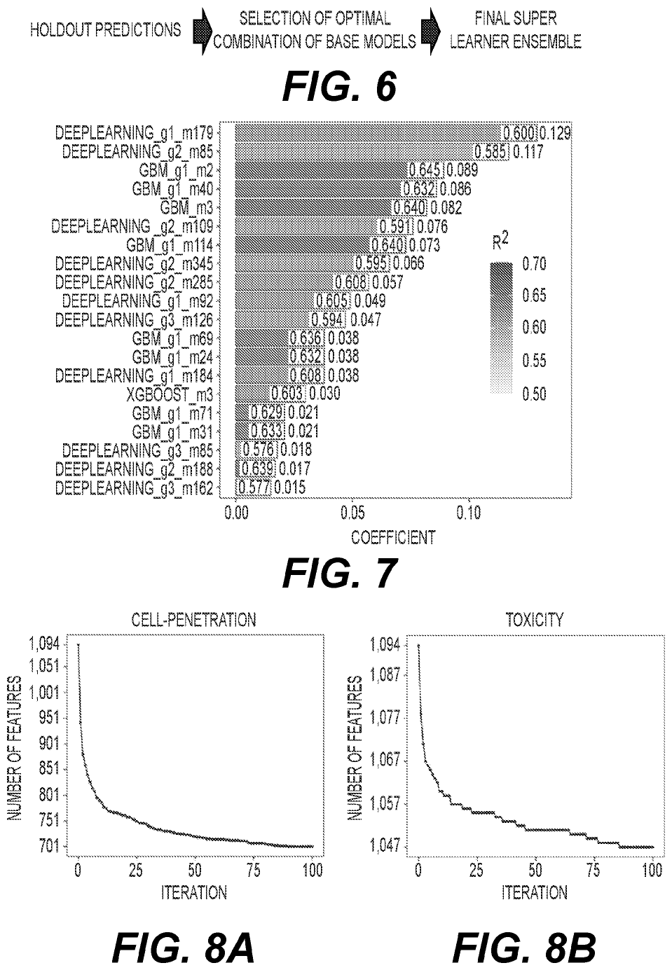

A regression SL ensemble with the reduced dataset was then built. SL is an ensemble model that combines different types of machine learning models (base models) by calculating their weighted contributions using another machine learning model called meta-learner (FIG. 6). The predictive performance of a super learner ensemble is assured to be at least as accurate as the best-performing base model (van der Laan, et al., Stat Appl Genet Mol Biol 6, Article 25, (2007), and van der Laan, et al., Report No. 30, (Division of Biostatistics, University of California; Berkeley, 2003)). The base models we used included deep learning (LeCun, Y., Bengio, Y. & Hinton, G. Nature 521, 436-444, (2015)), gradient boosting machine (GBM) (Friedman, J. H. Ann Statist 29, 1189-1232 (2001)), XGBoost (Chen, T. & Guestrin, C. Proc ACM SIGKDD Int (2016)), random forest (Breiman, L. Random forests. Mach Learn 45, 5-32 (2001)), extremely randomized trees (Geurts, P., Ernst, D. & Wehenkel, L. Mach Learn 63, 3-42 (2006)), and generalized linear model (Nelder, J. A. & Wedderburn, R. W. M. J R Stat Soc Ser A Stat Soc 135, 370-384 (1972)). The original SL algorithm used a meta-learner to calculate base model contributions and did not emphasize explicit base model selection.

Holdout predictions of peptides were generated for each base model with 10-fold cross validation on the input dataset. A meta-learner (using the non-negative least squares algorithm) was fitted on the holdout predictions with another 10-fold cross validation. The number of base models was reduced by applying an iterative model filtering procedure. The final super learner ensemble was trained on the input dataset with the optimal combination of the base models selected with 10-fold cross validation on the input dataset. Thus, an iterative filtering procedure was developed that systematically selected the optimal base model combination (FIG. 6), leading to improved performance and decreased prediction runtime. By applying the iterative base model filtering procedure, the number of base models in the SL was reduced from 891 to 24. The performance of the final SL ensemble improved to R²=0.69, which was higher than the best-performing base models of each algorithm family (Table 1). Further examination of constituent models showed that most of the base models in the final SL ensemble had the R² larger than 0.6 (FIG. 7). To explore other combinations of base models in the SL ensemble, SL with homogeneous base models consisting of models from only one algorithm family were constructed. It was found that the final regression SL ensemble with 24 models generated using the iterative filtering procedure had the highest R² and lowest mean absolute error (MAE) comparing to other SL ensembles (Table 1).

TABLE 1
Performance of super learner ensembles with different combinations
of base models and best-performing base models of each algorithm family
for the melanin binding regression dataset.

| | | Normalized | |
|---|---|---|---|
| Model | R² | MAE (%) | Model Type |
| Super learner ensemble 24 | 0.6879 | 2.3074 | Super learner |
| models (final) | | | ensemble |
| Super learner ensemble 48 | 0.6845 | 2.3142 | Super learner |
| models | | | ensemble |
| Super learner ensemble best | 0.6752 | 2.3817 | Super learner |
| of family | | | ensemble |
| Super learner ensemble all | 0.6672 | 2.3383 | Super learner |
| models | | | ensemble |
| Gradient boosting machine | 0.6558 | 2.4184 | Super learner |
| super learner ensemble | | | ensemble |
| Deep learning super learner | 0.6546 | 2.4039 | Super learner |
| ensemble | | | ensemble |
| XGBoost super learner | 0.6485 | 2.4059 | Super learner |
| ensemble | | | ensemble |
| Gradient boosting machine | 0.6447 | 2.4800 | Best base model of |
| | | | the algorithm |
| | | | family |
| Deep learning | 0.6402 | 2.5293 | Best base model of |
| | | | the algorithm |
| | | | family |
| XGBoost | 0.6027 | 2.6281 | Best base model of |
| | | | the algorithm |
| | | | family |
| Random forest | 0.5899 | 2.5716 | Base model |
| Extremely randomized trees | 0.5857 | 2.5986 | Base model |
| Generalized linear model | 0.3740 | 3.3394 | Base model |

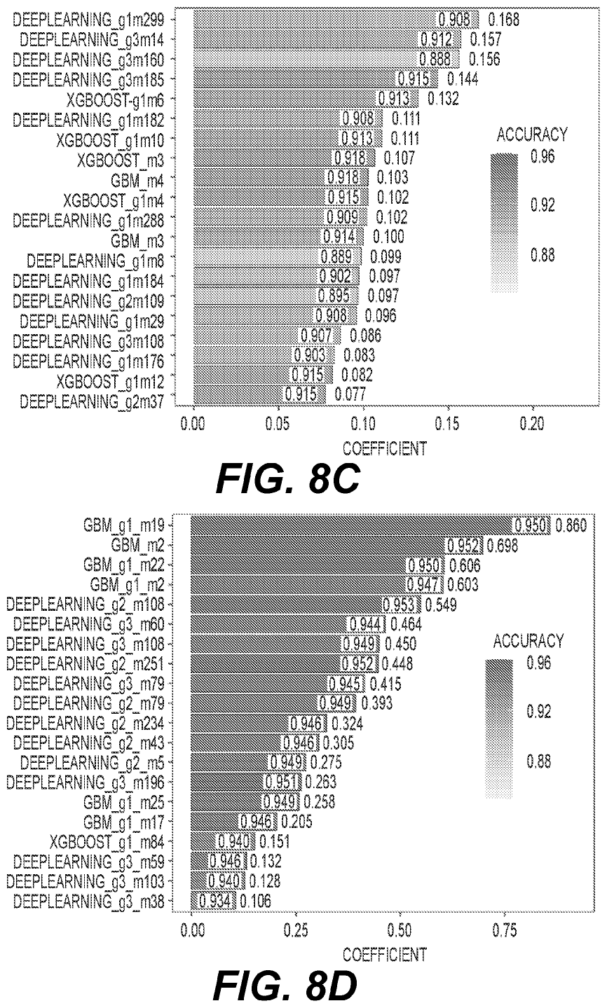

Next, cell-penetration and toxicity SL classification ensembles were built with peptides collected from the SkipCPP-Pred (Wei, L., Tang, J. & Zou, Q. BMC Genomics 18, 742, (2017)) and the ToxinPred (Gupta, S. et al., PLoS One 8, e73957, (2013)) databases, respectively. The same recursive feature elimination procedure was applied for data reduction. The features were reduced from 1,094 to 701 for the cell-penetration data set and from 1,094 to 1,047 for the toxicity data set after 100 iterations (FIGS. 8A-8B), and the prediction accuracies improved 0.009 and 0.001 for cell-penetration and toxicity, respectively. SL ensembles were subsequently trained and applied the iterative base model filtering method. The numbers of base models in the final SL ensembles were reduced from 1,000 to 118 for cell-penetration and from 1,000 to 28 for toxicity. While the best-performing base models of each algorithm family had relatively high performance, the final classification SL ensembles for both cell-penetration (Table 2) and toxicity (Table 3) had the highest model performance.

TABLE 2
Performance of super learner ensembles with different
combinations of base models and best-performing base models of each
algorithm family for the cell-penetration classification dataset.

| Model | Accuracy | Log-loss | Model type |
|---|---|---|---|
| Super learner ensemble 118 | 0.91871 | 0.2292 | Super learner |
| models (final) | | | ensemble |
| XGBoost | 0.91867 | 0.2129 | Best base model |
| | | | of the algorithm |
| | | | family |
| Deep learning | 0.9186 | 0.4002 | Best base model |
| | | | of the algorithm |
| | | | family |
| Gradient boosting machine | 0.9176 | 0.2129 | Best base model |
| | | | of the algorithm |
| | | | family |
| Super learner ensemble best | 0.9165 | 0.2074 | Super learner |
| of family | | | ensemble |
| XGBoost super learner | 0.9144 | 0.2250 | Super learner |
| ensemble | | | ensemble |
| Deep learning super learner | 0.9143 | 0.2424 | Super learner |
| ensemble | | | ensemble |
| Gradient boosting machine | 0.9143 | 0.2280 | Super learner |
| super learner ensemble | | | ensemble |
| Super learner ensemble all | 0.9111 | 0.2330 | Super learner |
| models | | | ensemble |
| Generalized linear model | 0.9045 | 0.2147 | Base model |
| Extremely randomized trees | 0.9002 | 0.2665 | Base model |
| Random forest | 0.9002 | 0.2983 | Base model |

Similar to the melanin binding regression SL models, when comparing to homologous SL ensembles, the final cell-penetration SL ensemble gave the highest accuracy and a comparable log-loss (Table 2), and the final toxicity SL ensemble had the higher accuracy and the lowest log-loss (Table 3). Most of the top contributing base models (top 20) in both cell-penetration and toxicity final SL ensembles had accuracy scores larger than 0.9 (FIGS. 8C-8D).

TABLE 3
Performance of super learner ensembles with different
combinations of base models and best-performing base models of each
algorithm family for the toxicity classification dataset.

| Model | Accuracy | Log-loss | Model Type |
|---|---|---|---|
| Super learner ensemble | 0.9573 | 0.1268 | Super learner |
| 24 models (final) | | | ensemble |
| Super learner ensemble | 0.9570 | 0.1279 | Super learner |
| 234 models | | | ensemble |
| Super learner ensemble | 0.9567 | 0.1550 | Super learner |
| all models | | | ensemble |
| Deep learning super | 0.9558 | 0.1608 | Super learner |
| learner ensemble | | | ensemble |
| XGBoost super learner | 0.9533 | 0.1504 | Super learner |
| ensemble | | | ensemble |
| Deep learning | 0.9527 | 0.8158 | Best base model |
| | | | of the algorithm |
| | | | family |
| XGBoost | 0.9526 | 0.1953 | Best base model |
| | | | of the algorithm |
| | | | family |
| Super learner ensemble | 0.9520 | 0.1373 | Super learner |
| best of family forest | | | ensemble |
| Gradient boosting | 0.9517 | 0.1477 | Best base model |
| machine | | | of the algorithm |
| | | | family |
| Gradient boosting | 0.9501 | 0.1486 | Super learner |
| machine super learner | | | ensemble |
| ensemble | | | |
| Extremely randomized | 0.9446 | 0.1624 | Base model |
| trees | | | |
| Random forest | 0.9408 | 0.1617 | Base model |
| Generalized linear | 0.9331 | 0.1674 | Base model |
| model | | | |

### Example 2: Peptide Generation for Super Learner Model Validation

### Materials and Methods

Amino acid frequencies at each position were calculated for the 5,499 melanin binding peptides used in the expanded peptide microarray, where the peptides were grouped into 8 sets based on normalized intensity ranges. For each intensity group, random peptides were simulated based on the position-dependent amino acid frequency. Melanin binding intensity values were predicted by the melanin binding SL ensemble. The selected peptide sequences were subsequently analyzed by the cell-penetration and toxicity SL ensembles for further classification. All peptides predicted as toxic were removed. In total, 127 peptides of length ranging from 7-12 were selected, including the TAT₄₇₋₅₇ peptide as the reference cell-penetration peptide.

### Peptide Synthesis

The library of C-terminal biotinylated 127 peptides used in cell culture experiments was synthesized by Gene Script using their Crude Peptide Library service. A terminal lysine was added to each peptide sequence to facilitate biotin conjugation. Peptides from the crude peptide library were further purified by being first dissolved in 50% Acetonitrile (ACN) with 0.1% TFA at 10 mg/nL. Shimadzu LC20 high-performance liquid chromatography (HPLC) system with Phenomenex reverse-phase preparative HPLC column (Gemini® 10 μm C18 110 Å, LC Column 250×21.2 mm, AXIA™ Packed) were used to separate and collect the peptides with an elution gradient of 5/5/90/90/5/5% solvent B (TFA 0.05% in ACN) at 0/2/10/12/13.5/15 min with a flow rate of 5 mL/min with monitoring 220 nm.

### In Vitro Melanin Binding Assay

Melanin nanoparticles (mNPs) were synthesized from the eumelanin of Sepia officinalis as previously described (Hsueh, H. T., et al., Nat Commun, 2023. 14: p. 2509). In brief, 10 mg/mL of eumelanin was suspended in DPBS using an ultrasonic probe sonicator (Sonics, Vibra Cell VCX-750 with model CV334 probe, Newtown, CT, USA) by pulsing 1 s on/off at 40% amplitude for 30 min in a 4° C. water bath. The suspension was then filtered through a 0.22 μm Millex-GV PVDF filter and transferred to PD-10 desalting columns. The resulting mNPs solution was lyophilized for 7 days and stored at −20° C. until further use. Sunitinib malate and HR97-sunitinib at a range of concentrations (12.5, 25, 50, 100 μg/mL) were dissolved in pH 6.5 PBS solution in 3 replicates. The solutions (400 μL) were then mixed thoroughly with 400 μL of 1 mg/mL mNPs in pH 6.5 PBS solution and transferred to the inner reservoir of the rapid equilibrium dialysis (RED) device inserts (8K MWCO). The outer reservoir was filled with 800 μL of pH 6.5 PBS solution. The samples were incubated on an orbital shaker with temperature controlled at 37° C. and 300 rpm for 48 h. The solutions from outer reservoir (free drug) were then collected and transferred to an autosampler vial for HPLC analysis (Prominence LC2030, Shimadzu, Columbia, MD) with the photodiode-array detection (PDA) system. Separation was achieved with a LUNA® 5 μm C18(2) 100 Å LC column 250×4.6 mm (Phenomenex, Torrance, CA) at 40° C. using isocratic flow. The amount of bound drug was used to calculate the binding capacity (moles drug/mg melanin) and the dissociation constant (Kd) as previously described (Kim, Y. C., et al., Drug Deliv Transl Res, 2022. 12(4): 826-837; Cheruvu, et al., Investig Ophthalmol Vis Sci, 2008. 49(1): 333-41)). The data point of HR97 conjugated to biotin for the high-throughput melanin binding screening assay (Hsueh, H. T., et al., Nat Commun, 2023. 14: 2509).

### Melanin Binding Assay for Super Learner Model Validation

The mNPs were mixed with C-terminal biotinylated peptides (10 μM) in pH 6.5 PBS solution and incubated in the rapid equilibrium dialysis (RED) 8 k device for 24 h on an orbital shaker at 900 rpm. A total of 10 μL of the solution from the rapid dialysis reservoir was collected. The concentration of unbound biotinylated peptides was analyzed with the Pierce™ Fluorescence Biotin Quantitation Kit. Four sets of melanin binding assays were performed with duplicate measurements per each assay. Melanin binding was calculated as the difference in free peptide amount normalized to the starting peptide concentration. In order to compare these values, which had a linear distribution, to those predicted by the SL model, the values were transformed to account for the exponential distribution of the fluorescent data from the peptide microarray. The values for mean percentage of peptide bound to mNPs were transformed using a simple log-linear regression model so that the values can be directly compared to predicted peptide bound to b-mNPs using the formula log₁₀(predicted peptide bound to b−mNPs)=−0.075+0.024×measured peptide bound to mNPs. The R² of the model was 0.78.

Cell-Penetration Assay with Induced Melanin ARPE19 Cell Type and Super Learner Model Validation

Four 96 well plates per group were seeded at 0.01×10⁶ cells/well. ARPE-19 cells were either cultured with DMEM:F12 medium containing 10% FBS according to protocol provided by the vendor (non-induced) or cultured in DMEM high glucose, pyruvate media with 250 PM of ferric ammonium citrate (Wolkow, N. et al., Exp Eye Res 128, 92-101, (2014)) for 2 months (induced) (Kim, Y. C. et al. Drug Deliv Transl Res, doi:10.1007/s13346-021-00987-6 (2021)). The expression of melanin was confirmed visually with bright field microscopy and by measuring absorbance at 475 nm (>0.4 a.u., not shown). Within each plate, 12 wells were randomly selected to quantify the cell numbers with an automated cell counter (Countess 3 Automated Cell Counter, Thermo Fisher) for normalization in the cell uptake study. Next, 100 μL (100 μM in pH 6.5 PBS) of each of the 127 C-terminal biotinylated peptides was added to n=3 wells for both the induced and non-induced ARPE-19 cells for 6 h at 37° C. The cells were then washed thoroughly 5 times with PBS solution to remove extracellular peptide. To quantify cell-associated peptides, the cells were lysed with 100 μL of RIPA lysis buffer at 4 C for 48 h. The concentration of intracellular biotinylated peptides was analyzed with the PIERCE™ Fluorescence Biotin Quantitation Kit. The mean intracellular concentration values were grouped by cell types (induced or non-induced), and the two-sample Wilcoxon test (Mann-Whitney) was calculated using the R function wilcox.test. Intracellular concentration values were also plotted against experimental melanin binding and the relationship was quantified using a simple linear model and its corresponding R².

### Shapley Additive Explanations (SHAP) Analysis of Multifunctional Peptide Predictions

To better characterize feature contributions to peptide property predictions, SHAP values for each of the 127 peptides were calculated using the function KernelExplainer from the Python package SHAP (version 0.39.0) (Lundberg, et al., A unified approach to interpreting model predictions, In Advances in Neural Information Processing Systems, Vol. 30 (2017)). The final super learner ensembles for melanin binding, cell-penetration, and toxicity were imported into the Python environment using the Python interface of H2O.ai, version 3.34.0.1 (H2O.ai. h2o:Python interface for H2O. (2021)). The Kernel SHAP method calculates feature importance with the local interpretable model-agnostic explanations (LIME) strategy (T., R. M., Singh, S. & Guestrin, C. Proc ACM SIGKDD Int (2016)), which approximates local predictions with linear models. The background dataset was generated by randomly selecting 100 instances from the original peptide property training set. SHAP values were calculated with the number of sampling times set at 1,000. Top 20 features ranked by mean absolute SHAP values were selected and visualized along with feature values normalized based on the original training data.

### Traceless Linker System for Conjugating HR97 to Brimonidine or Sunitinib.

The traceless linker system was designed to result in release of intact parent drug when triggered by an intracellular chemical and enzymatic event, such as protease cleavage of the amnide bond (Staben, L. R. et al. Nat Chen 8, 1112-1119, (2016)). Activation of the linker, MC-Val-Cit-PAB-OH, was conducted as previously reported with minor modifications (Staben, et al. Targeted drug delivery through the traceless release of tertiary and heteroaryl amines from antibody-drug conjugates. Nat Chem 8, 1112-1119, (2016)). MC-Val-Cit-PAB-OH (8.68 g, 15.2 mmol) was suspended in DMF (43.4 mL) at 0 C with water bath sonication for 30 min. After the solids were fully dispersed, thionyl chloride (1.22 mL, 16.7 mmol) was added dropwise. Following the addition, the reaction was held at 0 C for 45 min and then treated slowly with water (130 mL) to precipitate a yellow solid (MC-Val-Cit-PAB-Cl), which was collected by filtration. The solid was washed sequentially with water and MTBE and dried under the vacuum (30% yield). The brimonidine base or sunitinib base (1 eq) were combined with the MC-Val-Cit-PAB-Cl (1.1 eq) in DMF (0.25 M) at room temperature. Tetrabutylammonium iodide (0.5 eq) was added to the solution, followed by the addition of N,N-diisopropylethylamine (2.5 eq), and the mixture was stirred for 24 h. The mixture was diluted with 50:50 acetonitrile:water at 40-fold dilution for purifying the MC-Val-Cit-PAB-brimonidine (Mw=997.9) or MC-Val-Cit-PAB-sunitinib (Mw=954.2). A Shimadzu LC20 HPLC system coupled with photodiode array detector (PDA) and with Phenomenex reverse-phase preparative HPLC column (GEMINI® 10 μm C18 110 Å, LC Column 250×21.2 mm, AXIA™ Packed) were used to separate and collect the conjugates with an elution gradient of 10/90/90/10% solvent B (TFA 0.05% in ACN) at 1/11/13/15 min with a flow rate of 10 mL/min. The collected fractions were then transferred to the 20 mL scintillation vials and the Biotage V-10 solvent evaporator with Volatile mode was used to remove the acetonitrile. The solutions were further frozen and lyophilized (˜7% yield). Nuclear magnetic resonance (NMR) spectroscopy was used to confirm the presence of key functional groups in the products at each stage of the synthesis, including sunitinib base, Mc-VC-PAB-Cl, and Mc-VC-PAB-sunitinib. All compounds were dissolved in deuterated DMSO and characterized with a Bruker spectrometer (500 MHz). 1^{H} chemical shifts were reported in ppm (δ) and the DMSO peak was used as an internal standard. Data were processed using TopSpin NMR Data Analysis software, version 4.1.0, from Bruker. HR97 with cysteine at the C-terminus as the functional group for linker conjugation (FSGKRRKRKPRC, (SEQ ID NO: 1) MW 1.5 kDa, >97% purity from Thermo Fisher peptide custom service) was conjugated to the quaternary-ammonium-linked sunitinib (MC-Val-Cit-PAB-sunitinib) via a thiol-maleimide reaction. The MC-Val-Cit-PAB-sunitinib was first dissolved in 1 mL of PBS at 5 mg/mL, and the HR97 peptide powder (0.5 eq) was added. The solution mixture was adjusted to pH 7.4 and allowed to react for 2 h at room temperature. The solution was then added to 1 mL of acetonitrile and purified with the same prep-HPLC conditions. The collected fractions were transferred to 20 mL scintillation vials and a Biotage V-10 solvent evaporator with volatile mode was used to remove ACN. The solutions were lyophilized and stored at −20° C. (˜29% yield). For the sample preparation and MALDI-TOF analysis, the MALDI matrix sinapic acid (10 mg) was dissolved in 1 mL of acetonitrile in water (1:1) with 0.1% TFA, and 1 μL of sample (50 μM) was deposited on the MALDI sample plate. The matrix (2 μL, 10 mg/mL) was deposited on the air-dried sample and allowed to air dry for 10-20 min. The MALDI-TOF MS analysis was performed on a Bruker Voyager DE-STR MALDI-TOF (Mass Spectrometric and Proteomics core, Johns Hopkins University, School of Medicine) operated in linear, reflective-positive ion mode.

### In Vitro Stability Test for HR97-Brimonidine and HR97-Sunitinib Conjugate

Two pairs of human donor eyes were obtained from the Lions Gift of Sight under protocol TRB00056984 approved by the Johns Hopkins University School of Medicine Institutional Review Board. Both donors were male with a mean age of 74.5. The post-mortem times ranged from 35-40 hours. The eyes were kept at 4° C. during transport and arrived within 48 h post-mortem. The vitreous from each eye was isolated, combined, and filtered through a 0.02 μm syringe filter to remove cell debris. The aqueous was similarly combined and filtered. Each fluid type was aliquoted into 3 replicates (700 μL) and HR97-sunitinib (1 mg/mL) was added, and the mixture was incubated at 37° C. (n=3). On day 0, 1, 7, 14, 21, and 28, 100 L of the supernatant was collected, diluted with 900 μL of ACN, and characterized by HPLC (Prominence LC2030, Shimadzu) with Luna® 5 μm C18(2) 100 Å LC column 250×4.6 mm (Phenomenex, Torrance, CA). Separation was achieved with a Luna® 5 μm C18(2) 100 Å LC column 250×4.6 mm (Phenomenex) at 40° C. using isocratic flow (1 mL/min 60% TFA 0.1% in ACN). HR97-sunitinib was detected at λmax=420 nm (HR97-sunitinib retention time=1.9 min). The area under the curve (AUC) at day 0 was used to normalize the AUCs calculated at days 1, 7, 14, 21, and 28.

### Cathepsin Cleavage Assay for HR97-Brimonidine and HR97-Sunitinib Conjugate

An assay to demonstrate enzymatic cleavage of the linker was used as previously described with adaptations (Hsueh, et al., Nat Commun, 2023. 14: p. 2509; Staben, et al., Nat Chemistry, 2016. 8(12): p. 1112-1119). In brief, the HR97-sunitinib conjugate solution (200 M) was diluted with an equal volume of 100 mM citrate buffer at pH 5.5. Cysteine was added to a final concentration of 5 mM before the addition of human cathepsins B, K, L, and S (150 nM each). The mixture was then incubated from 0 h (control group) to 48 h at 37° C. The solutions were further diluted with ACN to 1 mL and the conjugate concentration was measured using the HPLC method described above. The concentration of HR97-sunitinib at 0 h was used to normalize the ratio remaining at later time points.

### Animals

All experimental protocols were approved by the Johns Hopkins Animal Care and Use Committee. All animals were handled and treated in accordance with the Association for Research in Vision and Ophthalmology Statement for Use of Animals in Ophthalmic and Vision Research. Equivalent numbers of both male and female animals were used. Brown Norway rats (6-10 weeks) were obtained from Harlan/Envigo. Dutch Belted rabbits (4-5 mo) were obtained from Robinson Services, Inc.

### Characterization of Drug Solubility

To measure solubility, 1 mg of sunitinib malate, sunitinib base, or HR-97-sunitinib was placed in microcentrifuge tubes with 0.2 mL of PBS. The samples were then placed on an orbital shaker (150 rpm) in an incubator at 37° C. After 7 days, samples were collected and centrifuged at 17,000 rcf for 30 min. The supernatant was collected, and concentrations were measured using HPLC as described above for sunitinib. Supernatant samples were mixed 1:10 with acetonitrile containing 0.1% trifluoroacetic acid. Acetonitrile and water were used as a mobile phase at a ratio of 55:45. Samples were eluted isocratically at a flow rate of 1 mL/min through a C18-reversed phase column at 40° C. UV absorbance was monitored at 431 nm.

### Rabbit IOP Measurements, Topical Dosing, and Intracameral Injection

For the IOP measurements in normotensive rabbits. Dutch Belted rabbits (2-3 kg) were used (n=5). IOP was measured with a hand-held rebound tonometer (TonoVet) in the awake and gently restrained rabbit. Each rabbit was acclimatized to the IOP measurement procedure for at least 5 d to obtain a stable background IOP reading. The average of three IOP measurements for an individual eye were taken every other day for 6 d (3 times in total) and used as a baseline value. For the ICM injection procedure, rabbits were anesthetized with ketamine/xylazine and received topical anesthesia with 0.5% proparacaine hydrochloride. A corneal pre-puncture was performed with a 30 G needle, followed with a single bolus intracameral injection of 200 μg HR-97 brimonidine or brimonidine in 100 μL saline using a 28 G needle. After the procedure, topical bacitracin-neomycin-polymyxin ophthalmic ointment was applied to both eyes to prevent infection and dry eyes. On day 7, an ophthalmologist inspected the HR97-brimonidine injected eyes to ensure there were no signs of toxicity. The lids, lashes and conjunctiva were normal, and the corneas were clear. The corneal endothelium was normal without any pigment deposition, the anterior chambers were all normal depth, and there was no apparent inflammation or fibrin strands in any of the eyes. The lenses were all clear and the iris pigmentation was symmetric. IOP was measured on days 2, 3, 4, 5, 6, 7, 8, 10, 12, 14, 16, 18, and 20 after the ICM injection and change in IOP from the baseline (ΔIOP) was reported. The average of three IOP measurements was taken for each eye. Alternatively, a single topical eye drop (ALPHAGAN® P 0.1%, 50 μL) was given (n=5). The IOP were measured immediately before the topical dosing (0 h), and at 2, 4, 6 and 8 h after the eyedrop administration.

### Rat Optic Nerve Head (ONH) Crush Glaucoma Model

Brown Norway rats (n=36 split into 3 groups of 12) received seven daily doses (5 μL) of 1 mg/mL sunitinib equivalent in HR97-SunitiGel (6.2 mg of HR97-sunitinib dissolved in 1 mL of 12% F127). The 12% F127 vehicle was previously described to undergo gelation on the ocular surface for prolonged ocular drug delivery (Kim, Y. C., et al., Drug Deliv Transl Res, 2022. 12(4): p. 826-837). The model was implemented similarly to investigate the timing of quantification of RGCs in the time-course study and the potential benefit of the HR97 peptide conjugation. In the time-course study, Brown Norway rats received the optic nerve head crush on day 0, and on day 1, 4, 7, 11, 14, or 19, the retinas were harvested and stained with DAPI and RBPMS for quantifying the remaining RGCs using the AI deep learning algorithm (n=6). In the HR97-SunitiGel RGC protection study, Brown Norway rats (n=36, split into 3 groups of 12) received seven daily doses (5 μL) of 1 mg/mL sunitinib equivalent in HR97-SunitiGel (6.2 mg of HR97-sunitinib dissolved in 1 mL of 12% w/w F127). The ONH crush procedure was then performed on separate groups of animals on day 0, 7, and 14 after the last dose. Another group of rats (n=12) received seven daily eye drops (5 μL) of water as a sham control prior to undergoing the ONH crush procedure on day 0 (immediately after the last dose). The third group of rats (n=12) received seven daily eye drops (5 μL) of 1 mg/mL equivalent sunitinib in SunitiGel (1.34 mg sunitinib malate in 1 mL of 12% w/w F127, equal to 1 mg/mL sunitinib equivalent). Rats received general anesthesia prior to topical anesthesia. Proparacaine hydrochloride (0.5% w/v) was applied topically to the right eye 1 min before the surgical process. The temporal conjunctiva of the left eye was grasped with 0.12 mm toothed forceps and incised parallel to the limbus with sharp iris scissors. The dissection was performed using two pairs of curved blunt-tipped forceps, and the orbital fat and soft tissue were retracted to expose the orbital portion of the optic nerve. The optic nerve was crushed at a position 1.5-2 mm posterior to the globe using reverse-action forceps for 10 s. The orbital soft tissue was then repositioned over the nerve and the conjunctiva was left to close by secondary intention (Kim, Y. C., et al., Drug Deliv Transl Res, 2022. 12(4): p. 826-837). After the procedure, topical bacitracin-neomycin-polymyxin ophthalmic ointment was applied to both eyes to prevent infection. Any animals that bled severely during the surgery were sacrificed and excluded from the study. Seven days after the optic nerve crush, rats were sacrificed for subsequent analyses.

### Retinal Ganglion Cell Staining and Imaging

Rats were sacrificed by cervical dislocation under general anesthesia. The eyes were then harvested and fixed with 4% paraformaldehyde for 2 h. The retinas were removed, incised for flat mounting, and post-fixed for 2 h. The retinas were then washed three times with PBS containing 0.5% Triton-100, and further incubated overnight at 4° C. in a solution containing or Goat anti-Rabbit IgG H&L Secondary Antibody Alexa Fluor 555 (Thermo Fisher) diluted 1:1,000 in PBS with 1% Triton X-100 and 1% bovine serum albumin. The retinas were again washed three times and incubated overnight in DAPI diluted 1:1,000 in PBS. The retinas were then washed with PBS containing 0.5% w/v Triton-100 for 30 min and incubated for 3 days at 4° C. in a solution containing rabbit anti-rat RBPMS antibody diluted 1:250 in PBS with 1% w/v Triton X-100 and 1% w/v BSA. The retinas were then washed for three times with PBS containing 0.5% w/v Triton-100 and incubated overnight at 4° C. in a solution containing Goat anti-Rabbit IgG H&L Secondary Antibody Alexa Fluor 555 (Thermo Fisher, Waltham, MA, USA) diluted 1:1000 in PBS with 1% w/v Triton X-100 and 1% w/v BSA. The retinas were washed again for three times and incubated overnight in DAPI diluted 1:1,000 in PBS. The resulting retinal wholemount was then mounted on a slide using Fluoromount-G. The prepared retinas were imaged with a Zeiss 710 Confocal Microscope. For each retinal wholemount, 16 images were taken from the region 2-3 mm from the optic nerve per each retinal quadrants using a 40× objective. The DAPI images were pseudo-colored in blue and the RBPMS images were pseudo-colored in red. The combined DAPI/RBPMS image layer was exported as a single JPEG format image and fed to an in house Faster_R_CNN Inception ResnetV2 deep learning object detection program for quantifying the RGCs. For training the TensorFlow Object Detection model (TensorFlow Version 1.2), 500 images labeled with 20,000 RGCs, and 100 images labeled with 5,000 RGCs were accomplished with the LabelImg function for the training and testing sets, respectively. Faster_R_CNN Inception ResnetV2 was used as the TensorFlow Object Detection model. The deep learning object detection program was specifically trained to recognize the RGCs. The cell numbers predicted by the model is highly consist with the those counted by human researchers (R²=0.99; n=3).

### Retinal Ganglion Cell Counting

For training the TensorFlow Object Detection model (TensorFlow Version 1.2), 173 images containing 4247 RGCs, and 62 images containing 1757 RGCs were manually labeled using the LabelImg function as the training and testing sets, respectively. Faster R-CNN with Inception Resnet v2 and SSD-MobileNet were used as the TensorFlow Object Detection models. The weighted sigmoid (sigmoid cross-entropy loss function) was used for the classification loss, and the weighted smooth L1 (box regression in object detection) was used for the localization loss. To avoid overfitting, the training process was terminated when the total loss was less than 1 or when the total loss has reached a steady state. An inference graph at each epoch was created with the script export_inference_graph.py provided in the object detection directory. The CellProfiler, version 3.1.9, with CellProfiler Analyst, version 2.2.1, was used in this study. The Metadata function was used to extract the DAPI and RBPMS sub-layer information from confocal images and the names were assigned using NamesAndTypes. The DAPI layers were first smoothed with the Gaussian filter function, and the IdentidyPrimaryObjects function was used to identify the DAPI areas using global and three-class Otsu threshold methods. The RBPMS sub-layers were smoothed with the Guassian Filter and then again smoothed with the Median Filter because the RBPMS staining with the RGCs had given uneven intensities and confused the program in the subsequent steps. The IdentifySecondaryObjects function with Propagation strategy and adaptive, three-class Otsu, and foreground methods were used to identify the RBPMS as secondary objects based on the DAPI areas identified in the primary object session. The object shape size, object intensity, and texture were measured for both DAPI and RBPMS objects as the features for CellProfiler Analyst, version 2.2.1. An SQLite database was generated after the CellProfiler had finished the feature extraction. In CellProfiler Analyst, RandomForestClassifier was used and RGCs in the unclassified window were dragged to the positive area following the suggested protocols. More than 100 cells in each positive and negative class were then manually assigned until the classification accuracy reached over than 90% computed using the evaluation function. Finally, the output scores were used to quantify the RGCs in each image. RGCs in another set of 200 images were manually counted by three researchers masked to the sample identity and the means were calculated for each image. In a rare instance that the cell count per image varied by more than 10%, the images were recounted by each person until the variance was <10%. Quantification results of the RGC images predicted by Faster R-CNN with Inception Resnet v2, SSD-MobileNet, and Cell Profiler Analyst models were compared to the manual counting results using Pearson correlation.

### Statistical Analysis of Model Performance

Retinal ganglion cells in two hundred images were counted by three researchers in a masked fashion before training the models with the object detection application programming interface (API). The counted results from the three masked researchers were averaged as the final result per each image. Quantification results of the RGCs predicted by the faster_rcnn_inception_resnet_v2_atrous_coco models were compared with the manual counting results using squared error, which estimated the model performance with mean and standard deviation. To assess the relationship between the best model prediction and the manual counting, the simple linear regression was used, and the coefficient of determination (R²) was calculated.

### Results

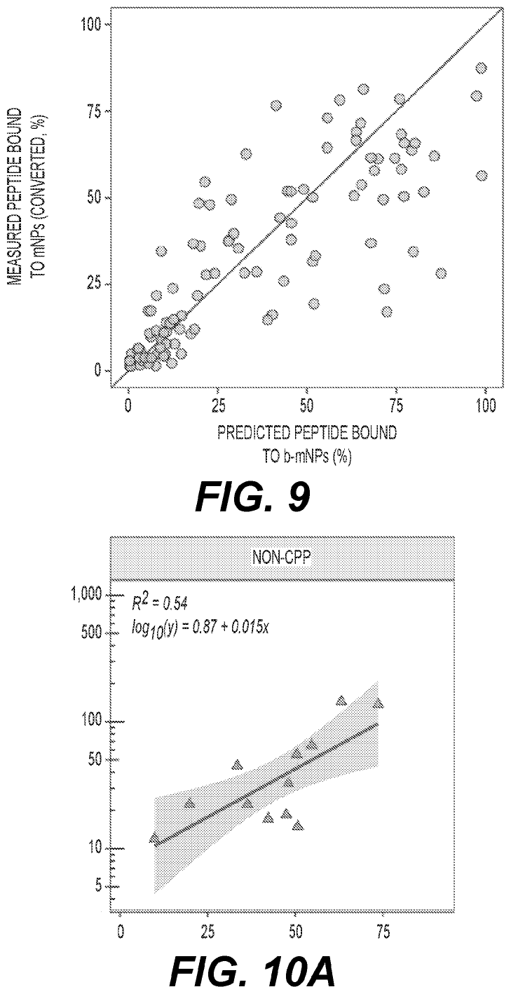

To validate the predictive accuracy of the final SL ensembles, it was next sought to experimentally determine the melanin binding and cell uptake of candidate multifunctional peptides. Peptides were generated using a position-dependent amino acid frequency matrix to tune the melanin binding from low to high (FIG. 1D). For each melanin binding level, random peptides were fed into SL ensembles for predicting melanin binding levels, as well as cell-penetration and toxicity properties. Peptides predicted to be toxic were removed, resulting in 127 peptides with length ranging from 7 to 12. For these selected peptides, 114 peptides were classified as cell-penetrating, and the remaining 13 peptides were classified as non-cell-penetrating. To experimentally measure melanin binding, an in vitro assay was utilized with mNPs and peptides that were biotinylated to facilitate fluorescent detection by Thermo Fisher fluorescence biotin quantitation kit (DyLight494 tagged avidin with HABA premix) (FIGS. 3A-3B). Since melanin binding values generated based on the peptide microarray system had an exponential distribution, the experimentally measured melanin binding values based on fluorescence were converted using a simple log-linear regression model to compare with melanin binding predictions. The R² of the model was 0.78, showing a high level of correlation between the predicted and converted experimental values (FIG. 9).

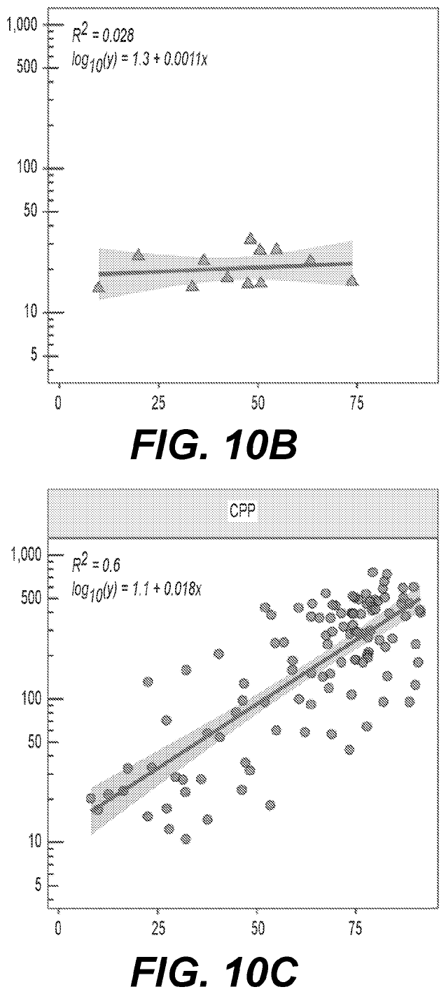

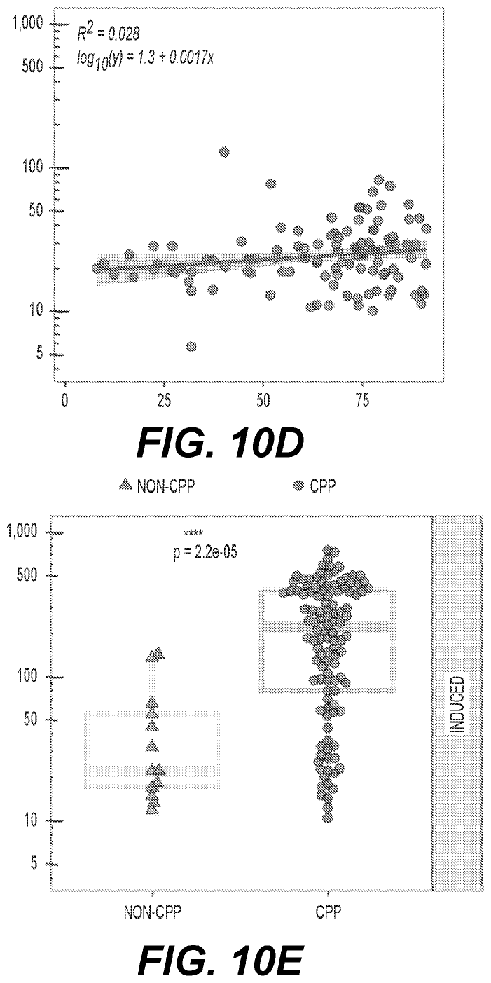

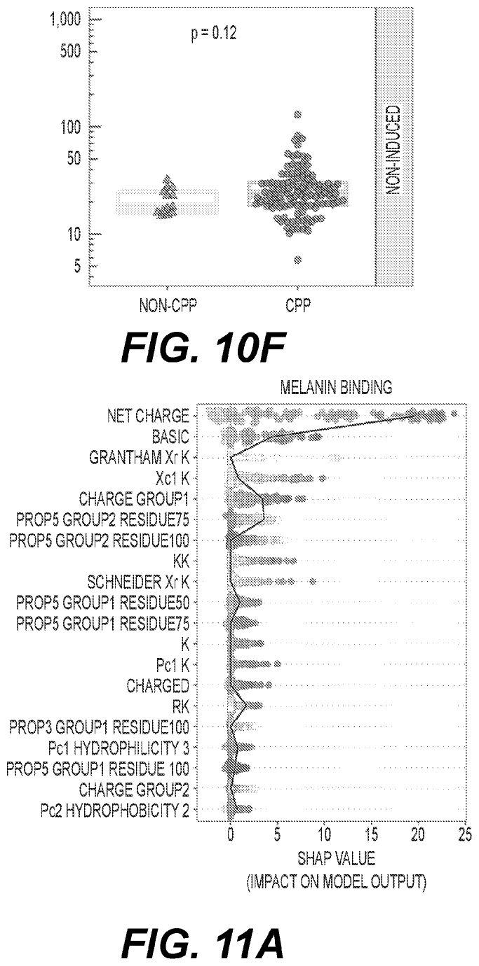

It was next characterized how the predicted cell-penetrating properties of the peptides affected cell uptake in a retinal pigment epithelium (RPE) cell line (ARPE-19). ARPE-19 cells were cultured using standard methods (non-induced, n=3) and using culture conditions that induce melanin production (induced, n=3) (Kim, Y. C. et al., Drug Deliv Transl Res, doi:10.1007/s13346-021-00987-6 (2021)). Peptides predicted to be cell-penetrating demonstrated significantly higher intracellular concentrations (median 220.8 pmol/100K cells) than those of non-cell penetrating peptides (median 22.4 pmol/100K cells) in the melanin-induced group (p=2.2 e-05, FIGS. 10A-10E). Similarly, peptides with higher predicted melanin binding levels were also observed to produce higher intracellular concentrations in induced cells (R²=0.6, FIGS. 10A-10E), suggesting a synergistic effect between the two properties.

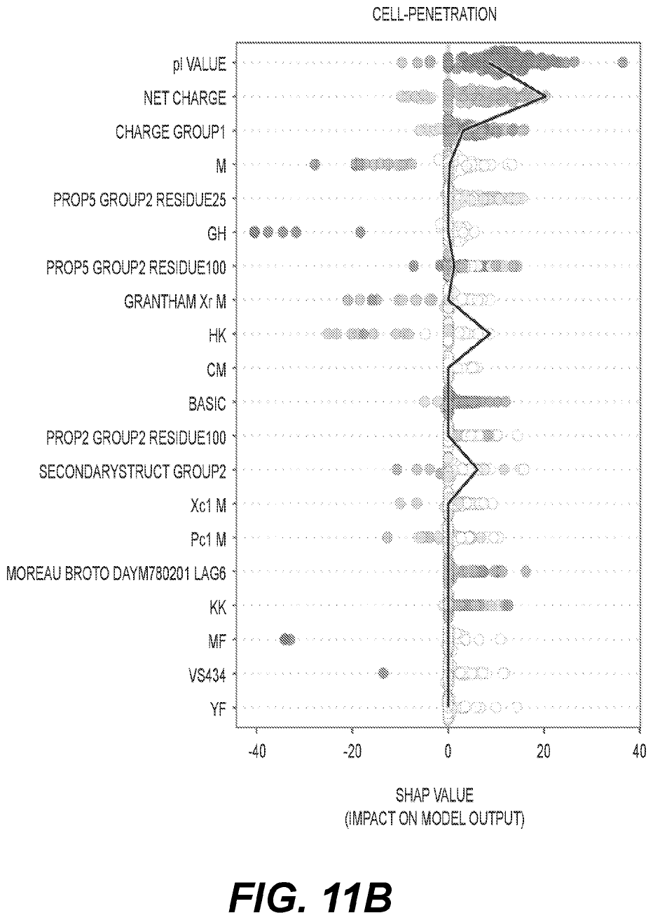

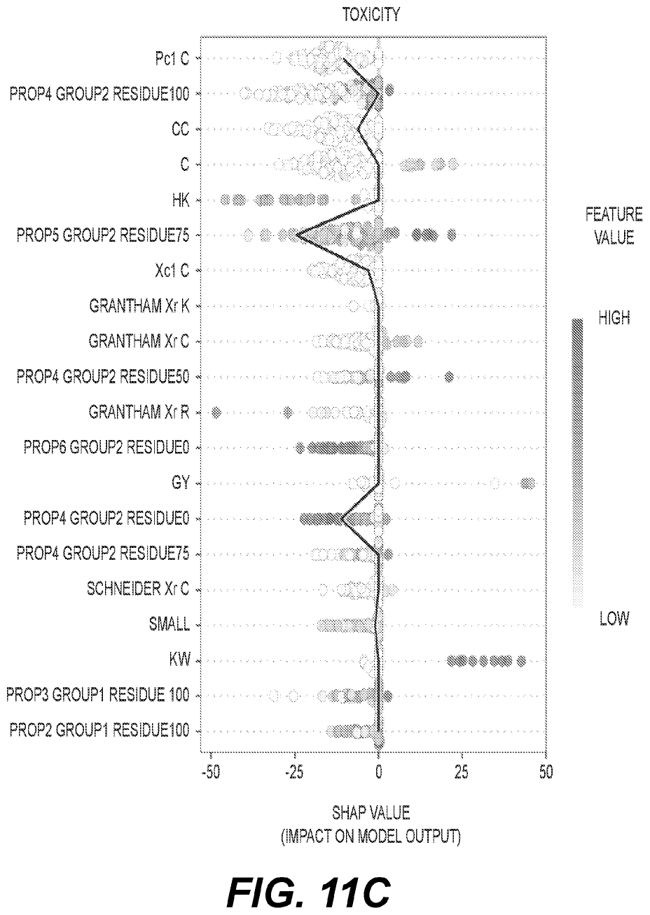

Shapley additive explanation (SHAP) analysis was then performed of the SL ensembles of the peptide properties based on the 127 candidate peptides to identify important features that have the most impact on the multifunctional peptide predictions. The result showed that more basic peptides with higher net charge likely bind more to melanin (FIG. 11A), which was consistent with the top important features calculated from the random forest classification model trained on the pilot peptide microarray. Similarly, peptides having higher isoelectric point (pI) and higher net charge may be predicted as cell-penetrating, while peptides containing less cysteines may be less toxic (FIGS. 11B-11C). A high melanin binding, cell-penetrating, and non-toxic candidate peptide, named HR97 (FSGKRRKRKPR, SEQ ID NO. 1) was selected for subsequent drug conjugation (FIGS. 11A-11C). HR97 out-performed the well-characterized cell-penetrating peptide fragment of the HIV trans-activator protein (TAT47-s, YGRKKRRQRRR) (SEQ ID NO. 2). HR97 demonstrated increased cell uptake compared to TAT₄₇₋₅₇ in both the induced (HR97=760-34.0, TAT₄₇₋₅₇=478±20.8 pmol/100K cells) and non-induced (HR97=82.5±15.8, TAT₄₇₋₅₇=58.7±7.43 pmol/100K cells) ARPE-19 cells.

A scheme was developed for conjugating HR97 to small molecule drugs for use in the eye. Brimonidine was first chosen, an IOP lowering drug that that is dosed topically three times per day. HR97 peptide was conjugated to brimonidine via a quaternary-ammonium traceless linker system and verified the structure of the purified conjugate by NMR.

Briefly, HR97 with cysteine at the C-terminus as the functional group for linker conjugation (FSGKRRKRKPRC, Mw=1519, >97% purity) was conjugated to the quaternary-ammonium-linked brimonidine (MC-Val-Cit-PAB-brimonidine) via a thiol-maleimide reaction (Compound I).

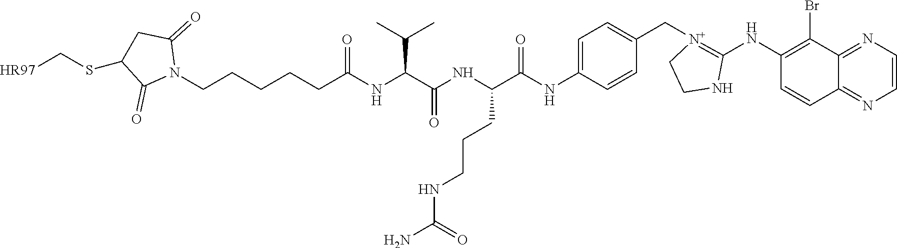

Compound I: HR97 with Cysteine at the C-Terminus Conjugated to Quaternary-Ammonium-Linked Brimonidine.

The MC-Val-Cit-PAB-brimonidine was first dissolved in 1 mL of PBS at 5 mg/mL. HR97 peptide powder (0.5 eq) was added directly to the solution. The solution mixtures were adjusted to pH 7.4 and allowed to react for 2 h at RT. The solution mixtures were then added to 1 mL of acetonitrile and purified with the same prep-HPLC conditions. The collected fraction solutions were first transferred to the 20 mL scintillation vials and the Biotage V-10 solvent evaporator with Volatile mode were used to remove the acetonitrile. The solutions were lyophilized and stored at −20° C. NMR was used to confirm the presence of key functional groups in the products of each stage of the synthesis, including brimonidine, Mc-VC-PAB-Cl, and Mc-VC-PAB-brimonidine. All compounds were dissolved in deuterated DMSO and ran with Bruker spectrometer (500 MHz). The prep-HPLC retention time (RT) of brimonidine, Mc-VC-PAB-brimonidine, and Mc-VC-PAB-Cl was 5.1, 9.8, and 11.4 min, respectively.

### Brimonidine

¹H NMR (500 MHz, DMSO-d₆) 8.84 (d, J=5 Hz, 1H), 8.68 (d, J=5 Hz, 1H), 7.83 (d, J=10 Hz, 1H), 7.55 (d, J=10 Hz, 1H) 6.54 (s, 2H), 3.40 (s, 4H).

### Mc-VC-PAB-Cl

¹H NMR (500 MHz, DMSO-d₆) 10.03 (s, 1H), 8.08 (d, J=7 Hz, 1H), 7.80 (d, J=8.5 Hz, 1H), 7.60 (d, J=8 Hz, 2H), 7.36 (d, J=8.5 Hz, 2H), 7.01 (s, 2H), 5.97 (bs, 1H), 5.41 (vbs, 1H), 4.71 (s, 2H), 4.38 (t, J=7.5 Hz, 1H), 4.18 (dd, J=1, 8 Hz 1H), 3.06-2.89 (m, 2H), 2.21-2.08 (m, 2H), 1.99-1.92 (m, 1H), 1.75-1.65 (m, 1H), 1.52-1.42 (m, 5H), 1.38-1.31 (m, 1H), 1.19 (pen, J=7.5 Hz, 2H), 0.86 (d, J=6.5 Hz, 3H) 0.85 (d, J=7 Hz, 3H).

### Mc-VC-PAB-Brimonidine

¹H NMR (500 MHz, DMSO-d₆) 9.98 (s, 1H), 9.21 (bs, 1H), 9.11 (d, J=1.5 Hz, 1H), 9.08 (d, J=2 Hz, 1H) 9.05 (d, J=1.5 Hz, 3H), 8.98 (s, 1H), 8.59 (bs, 5H), 8.15 (s, 1H), 8.12 (d, J=9 Hz, 5H), 8.07 (d, J=7.5 Hz, 2H), 7.84 (d, J=9 Hz, 4H), 7.81 (d, J=9 Hz, 1H), 7.71 (d, J=8.5 Hz, 1H), 7.60 (dd, J=4.5, 8.5 Hz, 1H), 7.52 (d, J=8.5 Hz, 2H), 7.22 (d, J=8.5 Hz, 2H), 6.53 (s, 3H), 6.40 (dd, J=3.5, 27 Hz, 2H), 6.25 (s, 1H), 6.22 (s, 1H), 6.06-6.00 (m, 3H), 5.42 (d, J=10.5 Hz, 5H), 5.15 (d, J=14.5 Hz, 2H), 4.81 (d, J=18.5 Hz, 2H), 4.33 (s, 1H), 4.25 (m, 2H), 4.13-4.07 (m, 2H), 4.01-3.96 (m, 1H), 2.96-2.82 (m, 4H), 2.17-2.03 (m, 4H), 1.87 (dd, J=6.5 Hz, 2H), 1.64-1.56 (m, 2H), 1.53-1.48 (m, 2H), 1.44-1.36 (m, 4H), 1.32-1.31 (d, J=6.5 Hz, 4H), 0.76 (d, J=6.5 Hz, 3H), 0.74 (d, J=6.5 Hz, 3H).

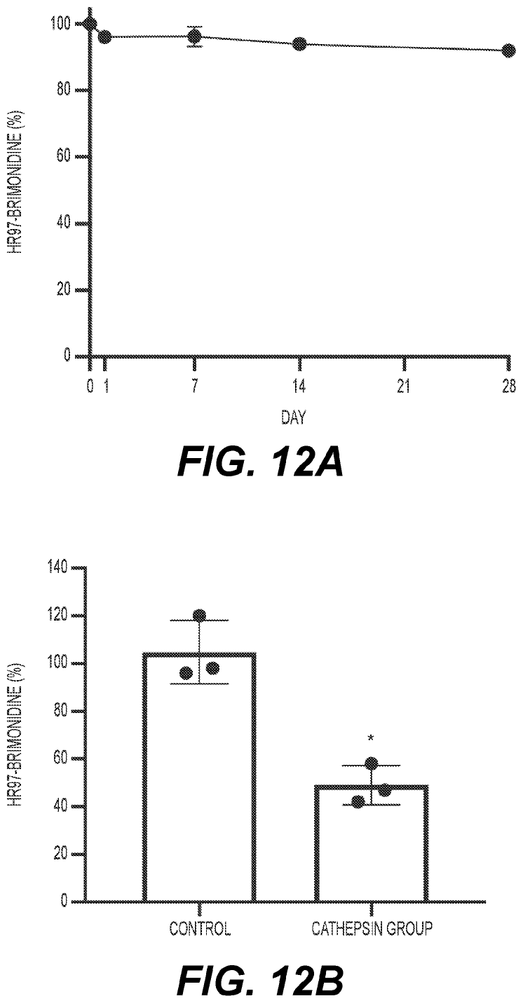

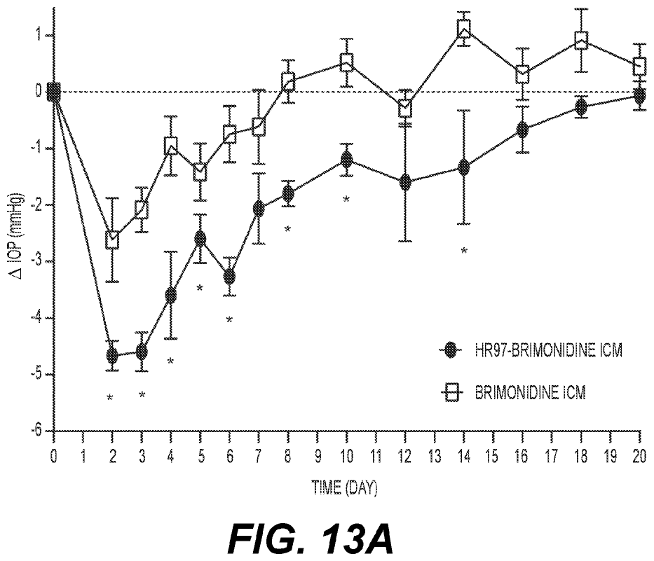

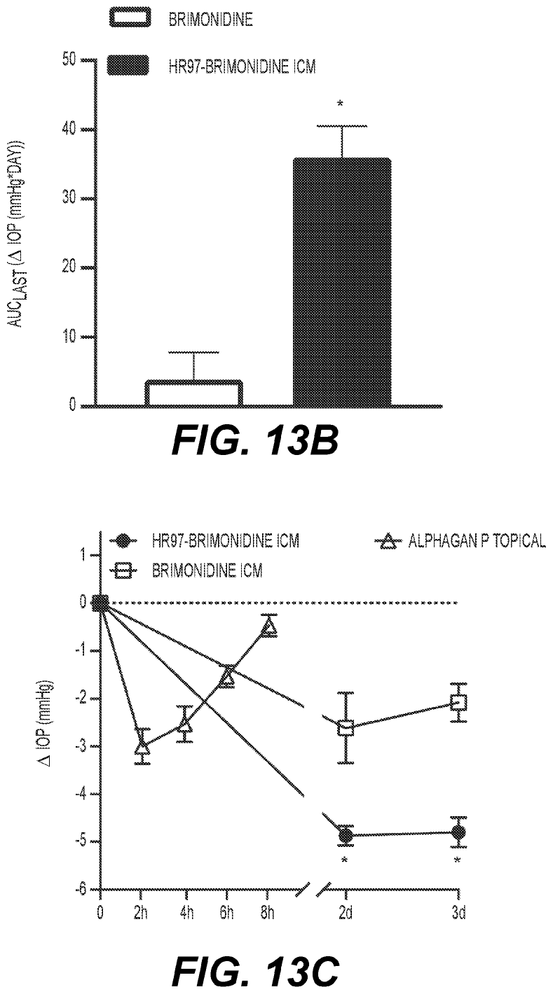

The traceless linker system was designed to remain stable until proteolytic cleavage inside cells (Staben, L. R. et al., Nat Chem 8, 1112-1119, (2016)). When incubated in human aqueous fluid, approximately 7% of the brimonidine was released from the HR97-brimonidine conjugate over 28 days in vitro (FIG. 12A). However, upon incubation with human cathepsin cocktails, 52% of the brimonidine was cleaved within 48 hours (FIG. 12B). A single intracameral injection of HR97-brimonidine conjugate or brimonidine (1 mg/ml based on the brimonidine mass) was next performed in normotensive Dutch Belted rabbits (n=5). Intracameral injection of the HR97-brimonidine conjugate resulted in a greater peak reduction in IOP from baseline (ΔIOP) compared to brimonidine at 2 days (−4.9±0.46 mmHg vs −2.6±1.65 mmHg, p<0.05) (FIG. 13A). The average ΔIOP in the HR97-brimonidine conjugate group was significantly larger than in the brimonidine group for up to 14 days (FIG. 13A). The time for the average ΔIOP to return to baseline was 20 days in the HR97-brimonidine conjugate group compared to 8 days in the brimonidine group (FIG. 13A). When summing the area under the curve (AUCₗₐₛₜ) for the cumulative ΔIOP over the 20-day measurement period, the HR97-brimonidine conjugate showed a ˜17-fold increased AUC compared to the brimonidine group (p<0.001) (FIG. 13B). In contrast, a single dose of a commercial brimonidine eyedrop, ALPHAGAN® P, resulted in a peak ΔIOP of only −3.0±0.82 mmHg and provided IOP lowering for only about 8 h (FIG. 13C).

Sunitinib is a protein kinase inhibitor with activity against dual leucine zipper kinase (DLK) and leucine zipper kinase (LZK). Inhibition of DLK and LZK by sunitinib leads to enhanced survival of retinal ganglion cells (RGCs) after optic nerve injury (Welsbie, D. S. et al., Proc Natl Acad Sci USA 110, 4045-4050, (2013); Welsbie, D. S. et al., Neuron 94, 1142-1154 e1146, (2017); and Watkins, T. A. et al., Proc Natl Acad Sci USA 110, 4039-4044, (2013)). HR97 was conjugated to sunitinib via the quaternary-ammonium traceless linker system (Compound II).

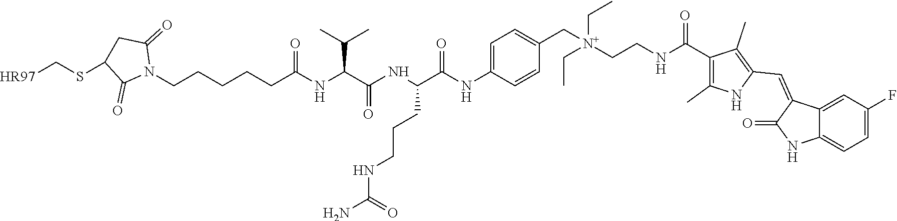

Compound II: HR97 with Cysteine at the C-Terminus Conjugated to Quaternary-Ammonium-Linked Sunitinib

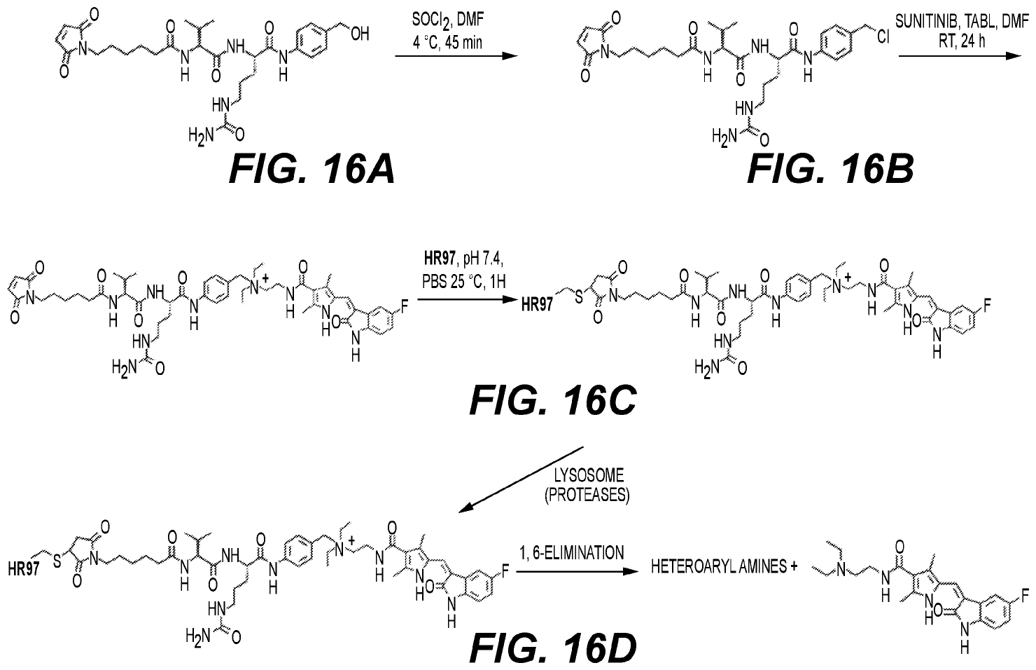

Briefly, HR97 peptide was conjugated to the quaternary-ammonium-linked sunitinib via the thiol-maleimide reaction Mc-VC-PAB-sunitinib, sunitinib, and Mc-VC-PAB-Cl were confirmed by NMR spectra. FIGS. 16A-16D is a scheme for the synthesis of HR07-Sunitinib. All compounds were dissolved in deuterated DMSO and ran with Bruker spectrometer (500 MHz). The prep-HPLC retention time (RT) of sunitinib, Mc-VC-PAB-sunitinib, and Mc-VC-PAB-Cl was 5.4, 8.3, and 11.4 min, respectively.

The NMR data for each compound were as follows:

### Sunitinib

¹H NMR (500 MHz, DMSO-d₆) 13.68 (s, 1H), 10.88 (s, 1H), 7.76 (d, J=9 Hz, 1H), 7.71 (s, 1H) 7.43 (bs, 1H), 6.92 (t, J=9 Hz, 1H), 6.85-6.83 (m, 1H), 3.30-3.25 (m, 2H), 2.60-2.52 (m, 6H), 2.41 (d, J=9.5 Hz, 6H), 0.98 (t, J=7 Hz, 6H).

### Mc-VC-PAB-Cl

¹H NMR (500 MHz, DMSO-d₆) 10.03 (s, 1H), 8.08 (d, J=7 Hz, 1H), 7.80 (d, J=8.5 Hz, 1H), 7.60 (d, J=8 Hz, 2H), 7.36 (d, J=8.5 Hz, 2H), 7.01 (s, 2H), 5.97 (bs, 1H), 5.41 (vbs, 1H), 4.71 (s, 2H), 4.38 (t, J=7.5 Hz, 1H), 4.18 (dd, J=1, 8 Hz 1H), 3.06-2.89 (m, 2H), 2.21-2.08 (m, 2H), 1.99-1.92 (m, 1H), 1.75-1.65 (m, 1H), 1.52-1.42 (m, 5H), 1.38-1.31 (m, 1H), 1.19 (pen, J=7.5 Hz, 2H), 0.86 (d, J=6.5 Hz, 3H) 0.85 (d, J=7 Hz, 3H).

### Mc-Vc-PAB-Sunitinib

¹H NMR (500 MHz, DMSO-d₆) 13.69 (s, 1H), 10.86 (s, 1H), 10.14 (s, 1H), 8.06 (d, J=5 Hz, 1H), 7.76 (t, J=6.0 Hz, 1H), 7.72-7.71 (m, 1H), 7.69 (s, 1H), 7.67 (d, J=3 Hz, 2H), 6.93 (s, 2H), 6.88 (m, 1H), 6.80-6.77 (m, 1H), 6.46 (bs, 2H), 5.92 (t, J=6 Hz, 1H), 5.34 (s, 2H), 4.45 (s, 2H), 4.32-4.28 (m, 1H), 4.11 (dd, J=7, 8.5 Hz, 1H), 3.67-3.63 (m, 2H), 2.98-2.85 (m, 3H), 2.40 (s, 3H), 2.38 (s, 3H), 2.15-2.01 (m, 2H), 1.92-1.85 (m, 1H), 1.68-1.58 (m, 1H), 1.59-1.49 (m, 1H), 1.47-1.37 (m, 6H), 1.33 (t, J=7 Hz, 7H), 1.17 (s, 5H), 1.144-1.080 (m, 3H), 0.78 (d, J=7 Hz, 3H), 0.75 (d, J=7 Hz, 3H).

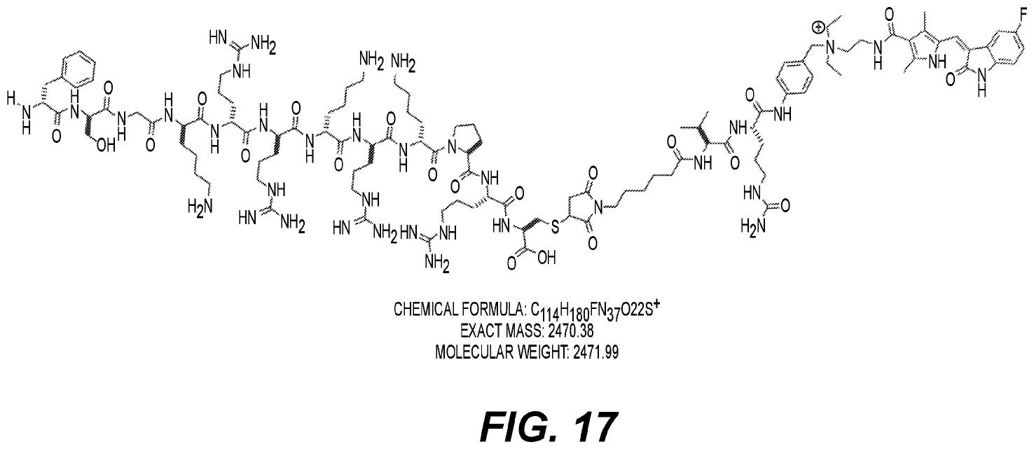

FIG. 17 shows the molecular structure of HR97-sunitinib. The m/z calculated for C₁₁₄H₁₈₀FN₃₇O₂₂S+ was 2,470.38, and the conjugate was measured at 2,511.11 [M-H+K]⁺.

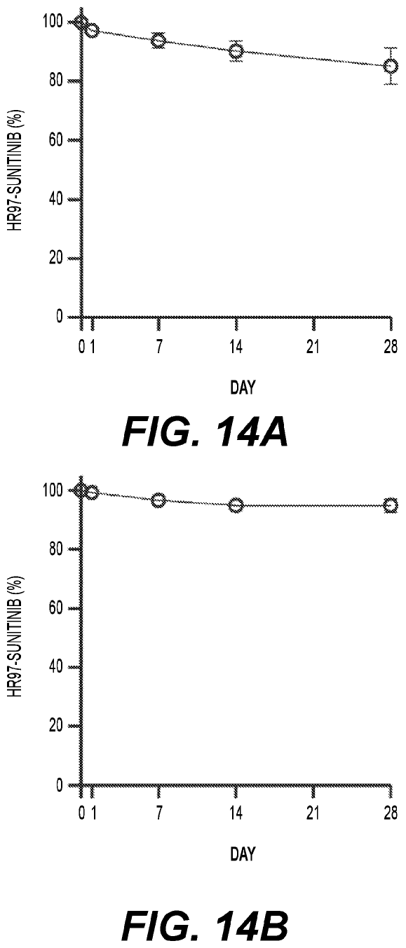

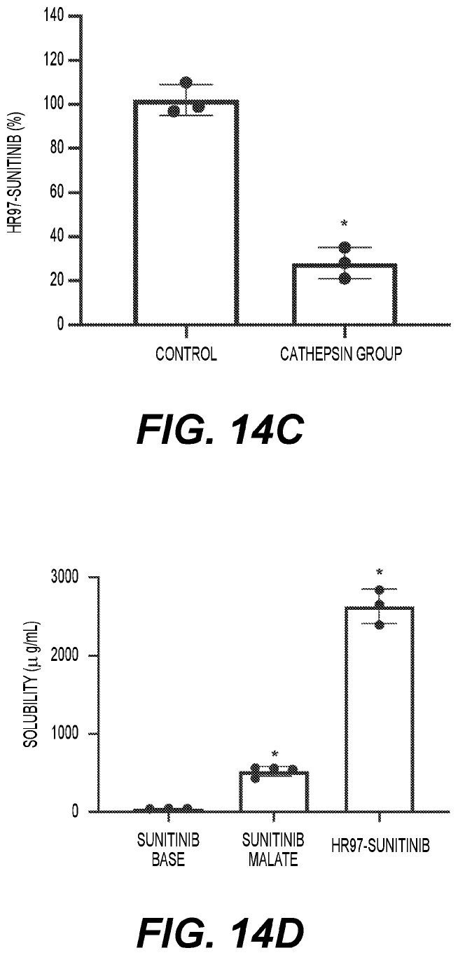

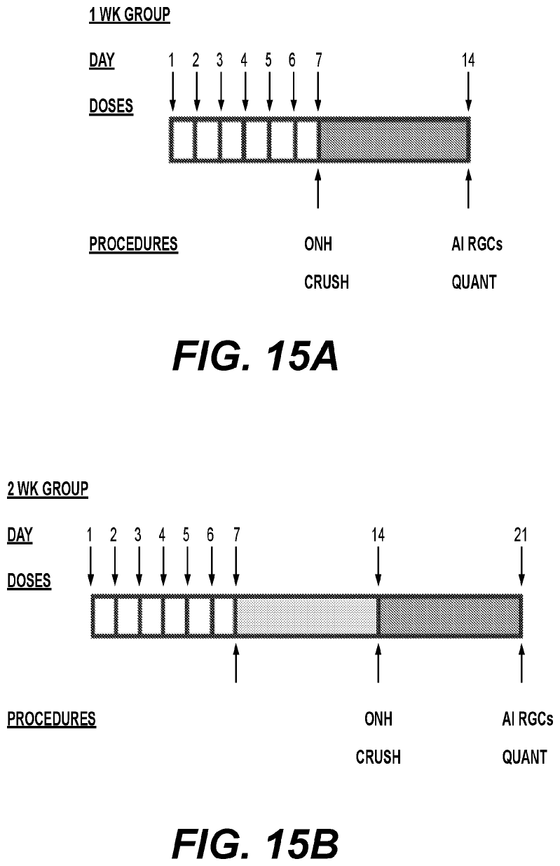

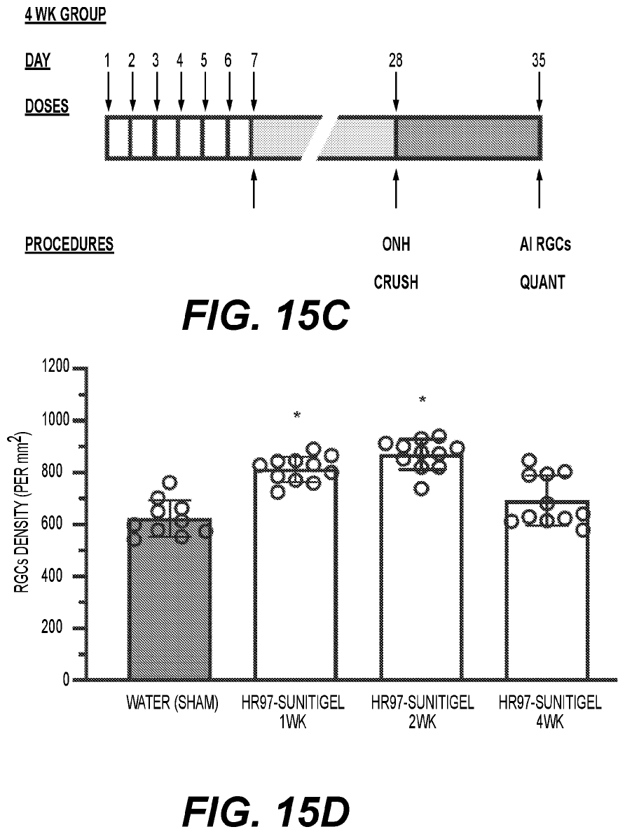

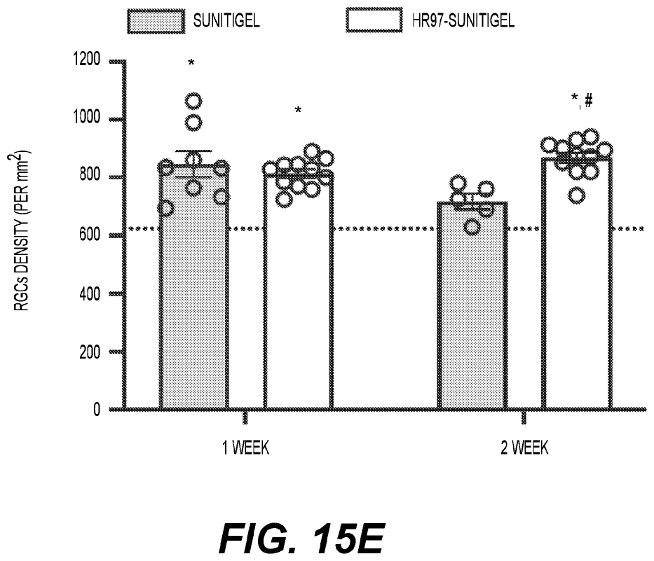

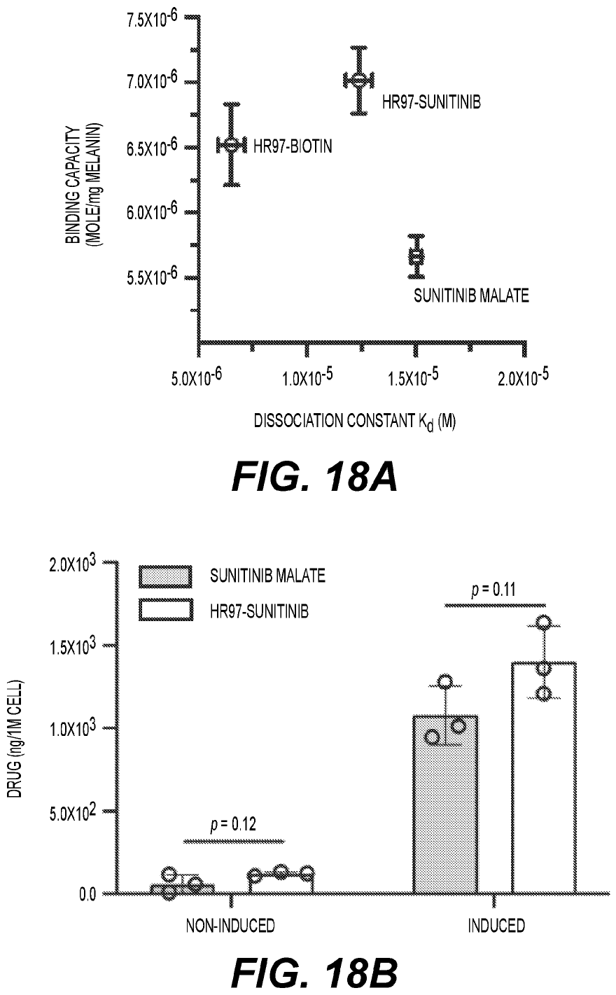

A scheme for conjugating HR97 to sunitinib via a quaternary-ammonium traceless linker system was developed and each intermediate product was structurally confirmed by NMR, HPLC, and MALTI. When the HR97 peptide conjugated to a drug that exerts therapeutic action in the posterior segment tissues was incubated in human aqueous fluid or vitreous fluid, approximately 5% (FIG. 14B) and 15% (FIG. 14A) of the sunitinib was released from the HR97-sunitinib conjugate, respectively, after 28 days in vitro (FIGS. 14A-14B). HPLC analysis of cathepsin cleavage assay of the HR97-sunitinib conjugate. HR97-sunitinib was incubated with human cathepsin cocktails (cathepsin). Peak separation was visualized for HR97-sunitinib+cathepsin (red line) along with HR97-sunitinib (black line) and sunitinib (blue line). HPLC was conducted with a Luna® 5 m C18(2) 100 Å LC column 250×4.6 mm (Phenomenex, Torrance, CA) at 40° C. using isocratic flow (1 mL/min 60% TFA 0.1% in ACN). Upon incubation with supraphysiological concentrations of human cathepsin cocktails to enzymatically cleave the linker, ˜72% of the sunitinib was cleaved within 48 hours (FIG. 14C). Since higher drug solubility could be an advantage in formulating eye drops, the solubility of HR97-sunitinib was tested. 5.5-fold higher solubility was observed compared to sunitinib malate and 56-fold higher than sunitinib base (FIG. 14D). Although sunitinib already shows relatively high intrinsic melanin binding properties compared to other ophthalmic drugs, conjugation to HR97 provided a 1.3-fold increase in melanin binding capacity compared to sunitinib in vivo (Kd 7.30×10⁻⁶ M vs. 5.51×10⁻⁶ M) (FIG. 18A). Additionally, HR97-sunitinib provided a 2.2-fold increase in cell uptake compared to sunitinib in non-induced ARPE-19 cells (FIG. 18B). When incubated with ARPE-19 cells induced to produce melanin, HR97-sunitinib provided a 1.4-fold increase in cell uptake compared to sunitinib (FIG. 18B).

The HR97-sunitinib conjugate was then dissolved in a hypotonic gel-forming vehicle (HR97-SunitiGel) that was previously demonstrated to provide enhanced intraocular drug absorption after topical dosing. The engineered peptide was then evaluated to see if it imparted longer-lasting therapeutic effects to sunitinib in a rat model of RGC injury. Brown Norway rats were dosed with HR97-SunitiGel or SunitiGel daily for 7 days, the optic nerve head crush procedure was performed on day 0, 7, or 21 after the last topical dose, and the RGC survival was characterized 7 days after the injury (FIGS. 15A-15C). The RGC quantification results computed by the cell counting program showed that the neuroprotective effect of HR97-SunitiGel lasted for at least 2 weeks after the last dose (869.2±58.86 RGCs/mm2 compared to sham, 623.7±70.39 RGCs/mm2, with the effect waning 4 weeks after the last dose (692.2±96.58 RGCs/mm2, (FIG. 15D). In contrast, SunitiGel provided significant RGC protection at 1 week (846.4±125.8 RGCs/mm2) compared to the sham group, with protection waning 2 weeks after the last dose (717.3±59.94 RGCs/mm2, (FIG. 15E).

### A Deep Learning Object Detection Model was More Accurate in Counting RGCs

A key aspect of assessing neuroprotective capacity involves counting RGCs in different regions of flat-mounted retina tissues. Manual cell counting can be time-consuming, so a reliable, automated image analysis method was developed. RGC images were used to train SSD-MobileNet and Faster R-CNN with Inception Resnet v2 models, both of which are often used in the object detection research.

A total of 173 images with 4247 manual labeled cells from both healthy and ONH crushed retinas were used to train the SSD-MobileNet and Faster R-CNN Inception ResNet v2 models. The same image sets were used as inputs to CellProfiler for generating the features. Simple linear correlations between automated and manual quantification of 200 RGCs 40× images were calculated and the Pearson correlation coefficient (r) are noted. A scatter plot of SSD-MobileNet (epoch 51,350), r=0.975, was significant with the p-value threshold of 0.0001 (two-tailed). A scatter plot of Faster R-CNN Inception ResNet v2 (epoch 13,683), r=0.993 was significant with the p-value threshold of 0.0001 (two-tailed). A scatterplot of the random forest classifier was used in CellProfiler Analyst, r=0.947, which is significant with the p-value threshold of 0.0001 (two-tailed).

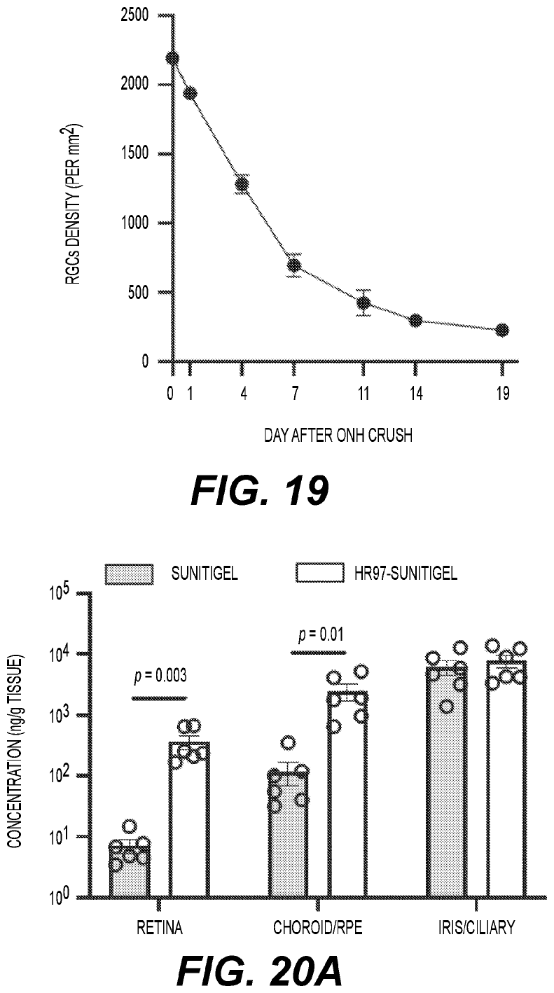

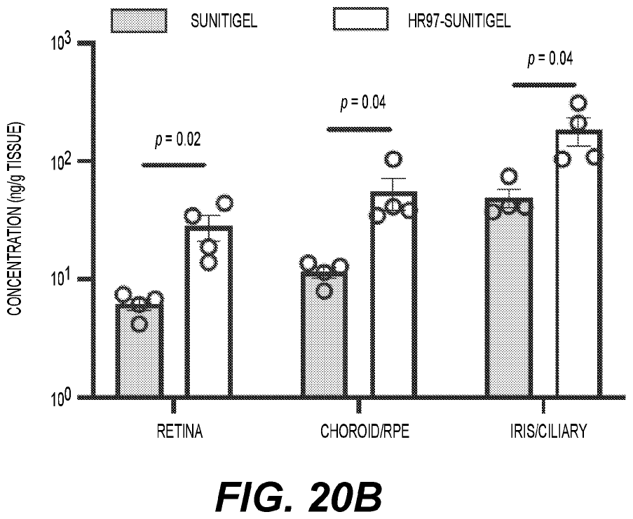

The Faster R-CNN with Inception Resnet v2 performed well in both high (>60) and low (<20) cell density image conditions (r=0.993), whereas the SSD-MobileNet slightly over-performed when the RGCs density was high in the images. The two object detection models were then compared to CellProfiler Analyst, a well-established open-source program for cell classification and recognition. The prediction results generated by CellProfiler Analyst were more vulnerable to the quality of the images, with lower prediction accuracies for both high and low cell density images compared to the deep learning object detection models (r=0.947) (FIG. 19). The capability of Faster R-CNN inception Resnet v2 in identifying the RGCs in various RGCs image conditions was further demonstrated, such as high and low cell density, oversaturated, and dim image settings. The Faster R-CNN with Inception Resnet v2 model (hereafter referred to as the cell counting program) was trained to assess retinal images collected from a time-course study of the rat ONH crush animal model to identify the optimal screening window for a neuroprotection drug delivery study. The quantification results using the cell counting program showed that the number of surviving RGCs decreased most rapidly between days 4 and 11 after the optic nerve head crush, and the curve started to flatten 11 days after the procedure (FIG. 19). Thus, a period of 7 days after the crush procedure was selected as the timeframe to assess RGC protection.

### Example 3: Pharmacokinetic Studies for HR97-SunitiGel

### Materials and Methods

### Pharmacokinetic Studies

Brown Norway rats (n=6) received once daily eye drops (5 μL) containing 1 mg/mL sunitinib equivalent in HR97-SunitiGel (6.2 mg of HR97-sunitinib dissolved in 1 mL of 12% w/w F127) or containing 1 mg/mL equivalent sunitinib in SunitiGel (1.34 mg sunitinib malate in 1 mL of 12% w/w F127, which was equal to 1 mg/mL sunitinib equivalent) for seven days. Fourteen days after the last dose, the iris, choroid, and retina were collected and analyzed for sunitinib concentration using LC-MS/MS. Similarly, Dutch Belted rabbits (n=4) received once daily eye drops (50 μL) containing 1 mg/mL sunitinib equivalent in HR97-SunitiGel or containing 1 mg/mL equivalent sunitinib in SunitiGel. Two hours after the last dose, the iris, choroid, and retina were collected for analysis of sunitinib concentration using LC-MS/MS.

### Measurement of Sunitinib in Ocular Tissues

Sunitinib concentrations in ocular tissues were measured by liquid chromatography-tandem mass spectrometry (LC-MS/MS) as previously described (Kim, Y. C., et al., Nat Biomed Eng, 2020. 4(11): p. 1053-1062). All samples were collected in pre-weighed tubes and stored at −80° C. until being processed for analysis. Tissue samples were homogenized in 100-600 μL 1×PBS using a Next Advance Bullet Blender before extraction. Sunitinib was extracted from 15-50 μL of tissue homogenates with 50 μL of acetonitrile containing 50/50/2.5 ng/mL of the internal standards (sunitinib-d10). The top layer was then transferred to an autosampler vial for LC-MS/MS analysis after centrifugation. All ocular tissue samples were analyzed using a 1×PBS standard curve for sunitinib. Separation was achieved with Waters Cortecs C18 (2.1×50 mm, 2.7 m). The column effluent was monitored using a Sciex triple quadrupol 4500 with electrospray ionization operating in the positive mode. The Mobile phase A was water containing 0.1% formic acid and the mobile phase B was acetonitrile containing 0.1% formic acid. The gradient started with the mobile phase B held at 10% for 0.5 min and increased to 100% within 0.5 min; 100% of mobile phase B was held for 1 min, and then the mobile phase B returned back to 10% and was allowed to equilibrate for 1 min. The total run time was 3 min with a flow rate of 0.3 mL/min. The spectrometer was programmed to monitor the following MRM transition 399.1→283.2 for sunitinib and 409.1→283.2 for the internal standard, sunitinib-d10. Calibration curve for sunitinib was computed using the area ratio peak of the analysis to the internal standard by using a quadratic equation with a x−2 weighting function over the range of 0.25-500, with dilutions up to 1:100 (v:v). Core technicians performing sample and data analysis were masked to the treatment groups.

### Statistical Analyses

Statistical analyses of two groups were conducted using two-tailed Student's t-test, two-tailed Mann-Whitney test, or two-way analysis of variance (ANOVA). For comparison of multiple groups, one-way ANOVA with Dunnett's multiple comparison test was used. Pearson correlation coefficients (r) and the corresponding p-values (two-tailed) were calculated to assess the relationships between model predictions and the mean values of the manual counting.

### Results

### HR97-SunitiGel Provided Increased Intraocular Residence Time in Rats and Therapeutically Relevant Drug Delivery to the Posterior Segment in Rabbits

Based on the improved efficacy of HR97-SunitiGel at the 2-week timepoint compared to SunitiGel, pharmacokinetic characterizations were conducted in rats to determine differences in intraocular drug concentrations. (FIG. 19B-19C). At week 2 after the last topical dose, HR97-SunitiGel provided 52-fold (362.2 ng/g vs. 7.0 ng/g), 21-fold (2430.0 ng/g vs. 116.4 ng/g), and 1.3-fold (7824.6 ng/g vs. 6093.5 ng/g) higher concentrations of combined sunitinib and N-desethyl sunitinib in the rat retina, choroid/RPE, and iris/ciliary body, respectively, compared to SunitiGel (FIG. 19C). To subsequently confirm that therapeutically relevant drug concentrations could be achieved in the larger eyes, rabbits were dosed once daily for seven days with HR97-SunitiGel or SunitiGel. At 2 hours after the last dose, HR97-SunitiGel provided 4.5-fold (27.8 ng/g vs. 6.1 ng/g), 4.7-fold (54.5 ng/g vs 11.5 ng/g), and 3.8-fold (182.8 ng/g vs 48.5 ng/g) higher concentrations of combined sunitinib and N-desethyl sunitinib in the rabbit retina, choroid, and iris, respectively, compared to SunitiGel (FIG. 19C). Importantly, the concentrations of sunitinib in the rabbit retina were comparable to concentrations found to be protective in the optic nerve crush model in rats. (Kim, Y. C., et al., Drug Deliv Transl Res, 2022. 12(4): p. 826-837).

### DISCUSSION AND CONCLUSION

Patient adherence is important in treatment of chronic ocular diseases such as glaucoma, wherein patients must chronically apply IOP lowering eye drops. Typically, only 40%-75% of patients adhere to glaucoma drop therapy regimens, even in scenarios where the patients know they are being monitored and were provided free medication. Failure to use medications as prescribed contributes to the progression of disease and can potentially lead to vision loss. A complementary strategy to IOP lowering, which is to directly target survival of the RGCs independent of IOP, was used. In this scenario, there is an added challenge of achieving effective drug delivery to the posterior segment with an eye drop, which is limited by ocular tissue barriers and uveal clearance. Designing a drug delivery system that can effectively deliver drugs to the posterior segment for neuroprotection, utilizing a non-invasive administration approach, and providing prolonged therapeutic effect to reduce dosing frequency may address several unmet needs in glaucoma management.

In these studies, by conjugating HR97 peptide to sunitinib, topical delivery of HR97-SunitiGel effectively protected RGCs for at least 2 weeks after the last topical dose. RGC identification and quantification are often employed in studies investigating cell and vision loss in glaucoma. The cell quantification has often been conducted manually by masked individuals hand-counting the cells or using the Image J software. Recently, the open-source CellProfiler and CellProfiler Analyst have received considerable attention for quantification of the cells because of its user-friendly interface, flexible analysis module, and integration of machine learning algorithms. Although CellProfiler provides an automatic pipeline for quantifying cells, the accuracy is heavily reliant on image quality. Commercial software, such as Metamorph (BioVision, Waltham, MA), Cellomics (Thermo Fisher), or TruAI deep-learning technology (Olympus) provide a convenient user interface and pre-designed modules to process and quantify cell images, but with annual subscription fees. The RGC quantifier developed in this study is based on the open source TensorFlow deep learning object detection system with the Faster R-CNN model. Although the model was trained on a relatively small image set, the cell counting program provided a high accuracy with increased flexibility to detect cells in images with varying cell densities and image qualities. Moreover, the model could be further trained with more confocal images in the future to accommodate different RGC staining qualities or expand to various systems of glaucoma animal models via transfer learning.

Overall, the development of innovative drug delivery systems that can overcome ocular barriers and enhance drug retention in the eye is essential for successful treatment of posterior segment diseases. These studies demonstrated that the HR97-sunitinib conjugate delivered via gel-forming eye drops (HR97-SunitiGel) provided increased sunitinib delivery to the posterior segment of rats and rabbits and prolonged neuroprotective effect for up to two weeks after the last dose in rats. The results demonstrate the benefits of increasing the melanin binding and cell penetration of small molecule drugs in the eye. The potential for developing topical eye drop delivery systems that can not only provide effective drug delivery to the posterior segment of the eye, but also require less frequent application, are of high value clinically and in improving patient quality of life.

Those skilled in the art will recognize, or be able to ascertain using no more than routine experimentation, equivalents to the specific embodiments of the invention described herein. Such equivalents are intended to be encompassed by the following claims.

1. A peptide for enhancing the delivery of an active agent to the eye, wherein the peptide binds to melanin, penetrates into cells, and is non-toxic.

2. The peptide of claim 1, wherein the peptide comprises an amino acid sequence comprising one or two phenylalanine, two amino acids selected from the group consisting of glycine and serine, and between four and twenty amino acids selected from the group consisting of lysine and arginine at a proportion of between 0.5 and 1.0.

3. The peptide of claim 1, wherein the peptide is between 5 and 12 amino acids, inclusive.

4. The peptide of claim 1, wherein the peptide comprises the amino acid sequence FSGKRRKRKPR (SEQ ID NO:1).

5. The peptide of claim 1 conjugated to an active agent for delivery to the eye,
wherein the amount of the active agent delivered to the eye by the peptide conjugate is greater than the amount of the active agent that is delivered to the eye without being conjugated to the peptide.

6. (canceled)

7. The peptide conjugate of claim 5, wherein the peptide comprises
an amino acid sequence of between 5 and 12 amino acids, inclusive.

8. The peptide conjugate of claim 5, wherein the peptide comprises the amino acid sequence FSGKRRKRKPR (SEQ ID NO:1).

9. The peptide conjugate of claim 5, wherein the active agent is selected from the group consisting of therapeutic agents, prophylactic agents, and diagnostic agents.

10. The peptide conjugate of claim 9, wherein the active agent is selected from the group consisting of proteins, nucleic acids, carbohydrates, and small molecules.

11. The peptide conjugate of claim 10, wherein the agent is selected from the group consisting of steroids, glaucoma agents, tyrosine kinase inhibitors, immunosuppressive agents, anti-fibrotic agents, anti-infectives, antioxidants that prevent oxidative and/or nitrosative damage, hormones, and chemotherapeutic agents.

12. (canceled)

13. The peptide conjugate of claim 11, wherein the active agent is selected from the group consisting of sunitinib, brimonidine, brinzolamide, cyclosporine A, moxifloxacin, budesonide, and acriflavine.

14. The peptide conjugate of claim 5, wherein the polypeptide comprises the amino acid sequence FSGKRRKRKPR (SEQ ID NO:1), and wherein the active agent is sunitinib.

15. The peptide conjugate of claim 5 in combination with a pharmaceutically acceptable excipient for administration to the eye.

16. The peptide conjugate of claim 15, formulated for topical, intracameral, subconjunctival and intravitreal administration.

17. The peptide conjugate of claim 15, comprising
a gel-forming polymer for application to the eye, formulated so that it is at a concentration below the critical gel concentration (CGC) of the polymer under isotonic conditions at a temperature between about 25° C. to about 37° C., and
excipients to form a pharmaceutically acceptable hypotonic formulation of the polymer suitable for delivery to the eye of an individual in need thereof.

18. The peptide conjugate claim 15, wherein the formulation is in dry or freeze-dried form.

19. The peptide conjugate of claim 17, wherein the gel-forming polymer is a thermosensitive gel-forming polymer such as a poloxamer.

20. The peptide conjugate of claim 19, wherein the thermosensitive gel-forming polymer has a critical solution temperature that is below 30° C.

21-22. (canceled)

23. The peptide conjugate of claim 19, wherein the gel-forming polymer is between greater than 12% and less than 24% F98 in an aqueous excipient.

24. The peptide conjugate of claim 19, wherein the gel-forming polymer is between 10 and 18% poloxamer F127.

25. The peptide conjugate of claim 17, wherein the gel-forming polymer forms a uniformly thick layer at the time of administration onto the ocular surface.

26-28. (canceled)

29. A method for treating an eye disease or disorder in a subject in need thereof, comprising administering to the eye of the subject the peptide conjugate of claim 15.

30. The method of claim 29, wherein the peptide conjugate is administered by a route selected from the group consisting of topical, intracameral, subconjunctival and intravitreal administration.

31. The method of claim 29, wherein the disease or disorder is selected from the group consisting of glaucoma, dry eye syndrome, macular degeneration, diabetic retinopathy, scleroderma, and cancer.

32. The method of claim 29, wherein the peptide conjugate is administered as an eye drop into the eye of the subject.

33. The method of claim 32, wherein the eye drop comprises an amount between about 10 μl and about 100 μl, inclusive, of the formulation.

34. The method of claim 29, wherein the peptide conjugate is administered as an injection into the eye of the subject.

35. The method of claim 29, wherein the method is repeated once or more.

36. The method of claim 35, wherein the method is repeated at a time selected from the group consisting of hourly, daily, every other day, every three days, every four days, every five days, every six days, weekly, every two weeks, or less often.

37. A method of screening for peptides having multiple desired functional properties, comprising
(i) screening the peptides for selective binding to a desired cellular target to identify one or more target-binding peptides;
(ii) generating a plurality of peptide variants based on the target binding peptides identified in (i) to create a first target-binding peptide library;
(iii) screening the first target-binding peptide library to identify a subset of target binding peptides;
(iv) screening the subset of target binding peptides for cell-penetration, and selecting a subset of cell-penetrating, target binding polypeptides; and
(v) screening the subset of cell-penetrating, target binding peptides for toxicity to identify a subset of non-toxic, cell-penetrating target binding polypeptides.

38. The method of claim 36, wherein the screening the peptides for selective binding in step i) comprises using an in vitro binding assay.

39. The method of claim 38, wherein the in vitro binding assay is an enzyme-linked immunoassay (ELISA), a phage display library, or a microarray system such as a high-throughput flow-based microarray system.

40. (canceled)

41. The method of claim 37, wherein the plurality of peptide variants in step ii) are randomly generated with a frequency of 5% for each of the 20 amino acids based on the target-binding peptides identified in i).

42. The method of claim 37, wherein the cellular target is melanin.

43. (canceled)

44. The peptide of claim 1 comprising the amino acid sequence FSGKRRKRKPR (SEQ ID NO:1), or one or more amino acid substitutions thereof.

45. The peptide of claim 1 comprising the amino acid sequence FSGKRRKRKPR (SEQ ID NO:1).


SEQUENCE LISTING (1 sequence)

SEQ ID NO: 1 - protein, 11 aa, synthetic construct
FSGKRRKRKPR